(19) United States
(12) Patent Application Publication
(10) Pub. No.: US 2023/0293678 A1
(43) Pub. Date: Sep. 21, 2023

(54) CATIONIC NANOPARTICLE ADJUVANTS

(71) Applicants: Aliasger K. SALEM, Coralville, IA (US); Sean M. GEARY, Iowa City, IA (US); Kareem EBEID, Iowa City, IA (US); Emad I. WAFA, Iowa City, IA (US); Rasheid SMITH, Iowa City, IA (US); University of lowa Research Foundation, Iowa City, IA (US)

(72) Inventors: Aliasger K. Salem, Coralville, IA (US); Sean M. Geary, Iowa City, IA (US); Kareem Ebeid, Iowa City, IA (US); Emad I. Wafa, Iowa City, IA (US); Rasheid Smith, Kalamazoo, MI (US)

(21) Appl. No.: 18/042,276

(22) PCT Filed: Aug. 20, 2021
(86) PCT No.: PCT/US2021/046962

§ 371 Date: Feb. 20, 2023

Related U.S. Application Data
(60) Provisional application No. 63/068,655, filed on Aug. 21, 2020.

(51) Int. Cl. A61K 39/39; A61K 39/00; C12N 15/86; A61P 35/00

(52) U.S. Cl. CPC A61K 39/39; A61K 39/001156; C12N 15/86; A61P 35/00; A61K 2039/55555; A61K 2039/572

(57) ABSTRACT

A cationic nanoparticle formed of a biodegradable or biocompatible synthetic polymer and a dendrimer having a diameter of about 125 nm to 1000 nm, and methods of using the nanoparticle, e.g., for delivery of an immunogen, are provided.

### CROSS REFERENCE TO RELATED APPLICATIONS

This application claims the benefit of the filing date of U.S. application No. 63/068,655, filed on Aug. 21, 2020, the disclosure of which is incorporated by reference herein.

### STATEMENT OF GOVERNMENT SUPPORT

This invention was made with government support under grant P30 CA086862 awarded by the National Cancer Institute. The Government has certain rights in the invention.

### BACKGROUND

Cancer immunotherapy represents an important treatment strategy for patients with inoperable cancers such as late-stage melanoma (Mougel et al., 2019). A primary goal of cancer immunotherapy is to break immune tolerance in the immunosuppressive tumor microenvironment and mount an efficient immune effector response against tumor cells (Hollingsworth & Jansen, 2019). Several FDA-approved cancer immunotherapy strategies exist for melanoma treatment including checkpoint blockade (Ipilimumab (anti-CTLA-4), Pembrolizumab (anti-PD1), oncolytic viral therapy (Talimogene laherparepvec) and combinational checkpoint inhibitors (Nivolumab-Ipilimumab) (Carreau & Pavlick, 2019). One approach which has had tremendous promise is the development of cancer vaccines. Cancer vaccines particularly in the therapeutic setting utilize adjuvants in combination with tumor-specific antigens (TSAs) or tumor-associated antigens (TAAs) to generate a cytotoxic T cell response against cancer cells (Hollingsworth & Jansen, 2019). Currently, Sipuleucel-T is the only FDA approved therapeutic cancer vaccine, and it is a dendritic cell (DC)-based cancer vaccine where the host DCs are incubated with a recombinant fusion antigen and then re-introduced into the patient. Despite its 4.1 month increase in median survival time and overall reduction in risk of death (Kantoff et al., 2010), the high cost (about $100,000 per patient) prohibits its widespread adoption. Currently, a personalized DC-based melanoma cancer vaccine is in a phase lib clinical trial (NCT02301611) but runs the risk of following the path Sipuleucel-T with having a high cost associated with it; thus, making it inaccessible to the majority of patients not adequately insured.

A viable alternative to cell-based vaccines are cancer vaccines that can be delivered without the need for harvesting tissue cells from the patient. For example, DNA-based delivery of TAAs or TSAs may reduce the high cost associated with these treatments. Viral vectors can deliver DNA based TAA or TSA and elicit a potent cytotoxic T lymphocyte (CTL) response (Warnock et al., 2011). While each viral vector has its advantages and disadvantages, the replication deficient adenovirus has been proven to have many advantages, and its few disadvantages can be surmounted easily. The replication deficient serotype 5 adenovirus (Ad5) has well-documented production techniques, can produce high viral titer stocks and encode relatively large DNA inserts which can result in multiple whole antigen inserts (Fougeroux & Hoist, 2017). Along with a high efficiency gene transfer, Ad5 has been shown to have a tropism for DCs, the most potent professional antigen presenting cell (APC) population (Veglia & Gabrilovich, 2017; Banchereau & Steinman, 1998). Given that DCs are the basis for all current cell-based cancer vaccines with FDA approval or those in clinical trials, an Ad5 cancer vaccine may be a viable and less expensive alternative (Kantoff et al., 2010; Fougeroux & Holst, 2017; Miller et al., 2002; Cheng et al., 2007). Ad5 has also been proven to be well-tolerated while being highly immunogenic in humans (Ledgerwood et al., 2010; Rajagopalan et al., 2002). An unfortunate downside to using viral vectors is the reduction of efficacy by neutralizing antibodies as a result of prior exposure from wildtype viruses; this can be circumvented using a gelatin matrix such as Gelfoam® to deliver the Ad5-based vaccines (Lou et al., 2011; Karan, 2017; Siemens et al., 2001).

Despite the promising potential demonstrated by Ad5-based cancer vaccines, there are disadvantages to employing them as a cancer treatment. Any vaccine, including a cancer vaccine, needs to mount an effective adaptive immune response to clear the offending entity. This response is carried out by the effector cells, namely CD8+ and CD4+ T cells along with B cells (Hollingsworth & Jansen, 2019) However, cancer vaccines encoding TAAs are starting against a severe disadvantage given that naïve T and B cells that have strong affinity for the TAA have been removed from the immune repertoire by central and peripheral tolerance mechanisms (Hollingsworth & Jansen, 2019; Pedersen et al., 2013; Buonaguro et al., 2011). In addition to this, they must overcome the myriad of immunosuppressive properties of the tumor microenvironment (TME) (Polak et al., 2009; Buonaguro et al., 2011). TSAs may be better at generating an immune response; however, TSAs lack the possibility of being prepared on a large standardized scale as they are often patient- or even tumor-specific; and there is still the issue of the immunosuppressive TME (Buonaguro et al., 2011; Pedersen et al., 2013). Numerous strategies have been tested in preclinical studies in an attempt to overcome these drawbacks and boost the immune response generated by TAA-based and model TSA-based cancer vaccines (Hollingsworth & Jansen, 2019).

### SUMMARY

The present disclosure relates to a cationic polymeric nanoparticle comprising a mixture of a synthetic biodegradable and/or biocompatible polymer, e.g., a copolymer such as PLGA, and a dendrimer, e.g., a dendrimer having an alkyl diamine core with tertiary amines such as PAMAM, and its use as an adjuvant, e.g., in a therapeutic vaccine such as a therapeutic cancer vaccine, a prophylactic vaccine, or in chemotherapy. In one embodiment, the combination of a synthetic biodegradable and/or biocompatible polymer and a dendrimer alone in a nanoparticle provides an adjuvant. The present disclosure also provides methods for stable storage of the nanoparticles after synthesis which may employ one or more cryoprotectants and/or diluents or carriers. In one embodiment, the cryoprotectant is a carbohydrate or polysaccharide, e.g., sucrose, trehalose, sorbitol, polyethylene glycol (PEG), hydroxyethyl starch, polyvinyl alcohol, glycerol, ethylene glycol, propylene glycol, triethylene glycol, polyvinylpyrrolidone, sucralose, mannitol, maltose, glucose, or cyclodextrins. In one embodiment, the composition comprising the nanoparticles is a vaccine, e.g., a cancer vaccine comprising nanoparticles encapsulating or complexed with a vector such as a plasmid encoding one or more tumor associated antigens (TAAs) or one or more tumor-specific antigens (TSAs). In one embodiment, the vaccine is a cancer vaccine comprising a recombinant virus, expressing one or more TAAs or one or more TSAs. In one embodiment, the vaccine comprises a recombinant adenovirus expressing one or more TAAs. In one embodiment the vaccine is co-administered with the nanoparticles. In one embodiment, the vaccine and the adjuvant are administered at two sites that are distant to each other. In one embodiment, the vaccine is administered before the adjuvant is administered, e.g., on the same day, or within 1 to 7, 14 or 21, or more, days. In one embodiment, the adjuvant is administered before the vaccine is administered, e.g., on the same day or within 1 to 7, 14 or 21 days. In one embodiment, the vaccine is locally administered, e.g., subcutaneously or intramuscularly administered. In one embodiment, the adjuvant is locally administered, e.g., intratumorally or peritumorally administered. As disclosed herein, the administration of the combination of the vaccine and the cationic nanoparticles resulted in a significant increase in TAA-specific cytotoxic T cells, significantly abrogated tumor growth and significantly extended survival of mice compared to mice treated with either component separately or in combination with CpG, which is a potent TLR-9 based adjuvant. In one embodiment, the nanoparticles have a diameter from about 150 nm to 1000 nm, e.g., from about 150 nm to about 250 nm, about 250 nm to about 350 nm, about 200 nm to about 300 nm, about 300 nm to about 400 nm, about 400 nm to about 500 nm, about 500 nm to about 600 nm, about 600 nm to about 750 nm, about 750 nm to about 900 nm in diameter.

In one embodiment, the disclosure provides for a method of preparing cationic polymeric nanoparticles comprising a mixture of one or more synthetic biodegradable and/or biocompatible polymers, e.g., a copolymer such as PLGA, and one or more dendrimers, e.g., a polyamidoamine, in a single solvent (diffusing phase), solvents including but not limited to acetone, acetonitrile, tetrahydrofuran, dimethylformamide, or chloroform. In one embodiment, the dendrimer is PAMAM, for example, formed of an ethylenediamine, 1,4 diaminobutane, 1,6-diaminohexane, 1,12-diaminododocane or cystamine core. In one embodiment, PAMAM has surface groups that include but are not limited to amine, amidoethanol, amidoethylethanolamine, or succinamic acid. In one embodiment, the dendrimer comprises different generations of PAMAM such as, for example, generation 3, 4, 5, 6, or 7. In one embodiment, the PAMAM core comprises ethylenediamine, cystamine, diamino hexane, diaminododecane, or diaminobutane. In one embodiment, the PAMAM surface is modified, e.g., modified with, for example, amino or amido, e.g., amidoethanol or amidoethanolamine surface groups, hexylamine surface groups, sodium carboxylate surface groups, mixed surface groups, succinamic acid surface groups, trymethoxysilyl surface groups, tris(hydroxymethyl)amidomethane surface groups or 3-carbomethoxypyrrolidinone surface groups. In one embodiment, the synthetic polymer is a copolymer, e.g., formed of different biodegradable polymers. In one embodiment, after synthesis, the nanoparticles may be lyophilized, e.g., after washing, optionally in the presence of a cryoprotectant or pharmaceutically acceptable carrier or diluent. Thus, a lyophilized product comprising the nanoparticles is also provided.

In one embodiment, the vaccine comprises a recombinant virus. In one embodiment, the recombinant virus is a recombinant adenovirus. In one embodiment, the recombinant virus is attenuated. In one embodiment, the recombinant adenovirus is one of species A-G. In one embodiment, the adenovirus is AdHu5, AdHu35, AdHu26, AdHu4 AdHu41, or Ad63.

In one embodiment, the nanoparticles are administered to a mammal in the absence of a vaccine, e.g., in the absence of a cancer vaccine.

In one embodiment, the nanoparticles are administered to a mammal in conjunction with another agent, e.g., the nanoparticles are employed as an adjuvant, e.g., with other immunomodulatory agents. In one embodiment, the other agent is a therapeutic agent. In one embodiment, the other agent is a cancer vaccine. In one embodiment, the other agent is a prophylactic vaccine, e.g., which may be administered to the same site. In one embodiment, the other agent is a chemotherapeutic agent, e.g., a drug or antibody. In one embodiment, the nanoparticles are combined with other cancer immunotherapy modalities including but not limited to: adoptive cell therapy with either tumor infiltrating leukocytes (TIL), chimeric antigen receptor (CAR) T cell or T cells with modified T cell receptors; or oncolytic viral therapy. In one embodiment, the nanoparticles comprise one or more other agents.

In one embodiment, the synthetic polymer is a polyolefin, e.g., polyethylene, polypropylene, polytetrafluoroethylene, or polyvinylchloride; a silicone, e.g., polydimethylsilane; a polyacrylate, e.g., polymethyl methacrylate or polyhydroxyethyl methacrylate; a polyester, e.g., polyethylene terediphthalate, a polyanhydride, PGA, PLLA (poly-L-lactic acid), PDLA (poly D-lactic acid), or polydioxanone; a polyether, e.g., polyether ketone or polyether sulfone; a polyamide; a polyurethane; or a sulfenamide-based polymer, e.g., poly(diaminosulfide).

In one embodiment, the dendrimer comprises PAMAM (polyamidoamine), poly-L-lysine, polyethyleneimine (PEI), e.g., linear or branched, POPAM (polypropyleneimine; PPI), or diethylaminoethyl dextran.

In one embodiment, the nanoparticle comprises one or more cationic lipids, including but not limited to, cationic lipids such as: DOPE, DOTMA: N[1-(2,3-dioleyloxy) propyl]-N,N,N-trimethylammonium chloride, DOTAP: 1,2-dioleyl-3-trimethylamonium-propane, DMRIE: N-(2hydroxyethyl)-N,N-dimethyl-2,3-bis(tetradecyloxy-1-propananium bromide), DOTIM: 1-[2-dioleoyloxy)ethyl]-2-oleyl-3(2-hydroxyethyl)imidazolinium chloride, DOGS: dioctadecylamidoglycylspermine, DC-Chol: [N—(N0,N0dimethylaminoethane)-carbamoyl]cholesterol, BGTC: bis-guanidium-tren-cholesterol, or DOPE: 1, 2-dioleyl-sn-glycerol-3phosphoethanolamine.

In one embodiment, the nanoparticle comprises DOTMA (N[1-(2,3-dioleyloxy) propyl]-N,N,N-trimethylammonium chloride,) DOTAP (1,2-dioleyl-3-trimethylamonium-propane), DMRIE (N-(2hydroxyethyl)-N,N-dimethyl-2,3-bis(tetradecyloxy-1-propananium bromide), DOTIM (1-[2-dioleoyloxy)ethyl]-2-oleyl-3(2-hydroxyethyl)imidazolinium chloride), DOGS (dioctadecylamidoglycylspermine), DC-Chol ([N—(N0,N0dimethylaminoethane)-carbamoyl]cholesterol), BGTC (bis-guanidium-tren-cholesterol), or DOPE (1, 2-dioleyl-sn-glycerol-3phosphoethanolamine), or a combination thereof.

In one embodiment, a cancer vaccine. e.g., expressing a TAA or TSA for one of the following cancers, is employed to prevent, inhibit or treat cancers including but not limited to carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. In one embodiment, the cancer vaccine, e.g., expressing a TAA or TSA for one of the following cancers, is employed to prevent, inhibit or treat cancers including but not limited to adrenocortical carcinoma; bladder urothelial carcinoma; cervical or endocervical cancers; cholangiocarcinoma; colon adenocarcinoma; lymphoid neoplasm diffuse large B cell lymphoma; glioblastoma multiforme; head and/or neck squamous cell carcinoma; kidney chromophobe; brain lower-grade glioma; liver hepatocellular carcinoma; lung adenocarcinoma; lung squamous cell carcinoma; mesothelioma; pancreatic adenocarcinoma; phaeochromocytoma or paraganglioma; rectum adenocarcinoma; skin cutaneous melanoma; thymoma; uterine corpus endometrial carcinoma; or uveal melanoma

In one embodiment, the vaccine encodes or includes a TAA, e.g., directed to HER2, PSA, TRP-2, EpCAM, GPC3, mesothelin, EGFR, CEA, or MUC1, an antigen encoded by cancer-gonad genes, embryonic/differentiation genes or overexpressed antigens. In one embodiment, the TAA is derived from proteins, such as WT1, NY-ESO-1, PRAME, Proteinase 3, or MAGE-A3. In one embodiment, the TAA is from a cancer-germline gene, e.g., a melanoma-antigen encoding (MAGE) gene, or a gene encoding BAGE, GAGE, LAGENY-ESO1, or SSX. Other TSAs useful in the vaccines include but are not limited to MOK (RAGE-1), PRAME, the inhibitor of apoptosis protein survivin, the wild-type p53 protein, or ERBB2 (HER2/NEU).

In one embodiment, the vaccine encodes or includes a TSA, e.g., a viral-derived cancer antigens (for example, human papillomavirus (HPV) and Epstein-Barr virus; or those generated from point mutations, mutational frameshifts, splice variants, gene fusions, endogenous retroelements and other classes, such as human leukocyte antigen (HLA)-somatic mutation-derived antigens and post-translational TSAs. an antigen encoded by cancer-gonad genes, embryonic/differentiation genes, overexpressed antigens, or viral antigens.

### BRIEF DESCRIPTION OF FIGURES

FIG. 1. Diagram showing the different stages of the synthesis of PMG5 nanoparticles.

FIGS. 2A-2F. Effect of lyophilization (LYO) with and without sucrose on PMG5 size, size distribution 337 and net surface charge. (A and B) TEM images of PMG5 at different magnifications (negative stain, 1.5% phosphotungustic acid). (C) Scanning electron photomicrograph of PMG5 (scale bar=3 μm while the scale bar on inlet image=100 nm). (D) Graph showing the hydrodynamic diameter of various groups. (E) Graph showing the size distribution (polydispersity index, PDI) of various groups. (F) Graph showing the net surface charge (zetapotential) of various groups. Data are plotted as mean±SD.

FIGS. 3A-3D. Evaluation of the three PM formulations with three different particle sizes. (A) Graph showing the size. (B) Graph showing the size distribution (polydispersity index, PDI). (C) Graph showing the net surface charge (zetapotential). (D) Graph showing the uptake of the three formulations in BMDCs. Data are plotted as mean±SD.

FIGS. 4A-4C. Evaluation of the uptake of PMG5 in BMDCs. (A) Graph showing the effect of lyophilization conditions on the uptake of PMG5 in BMDCs. (B) Graph showing the dose-uptake relationship of PMG5 in BMDCs. (C) Graph showing the effects of various inhibitors on the uptake of PMG5 in BMDCs. All experiments were performed using small sized PMG5. Data are plotted as mean±SD.

FIGS. 5A-5D. Thermal Stability of PMG5. (A) Differential Scanning Calorimeter (DSC) thermogram of PMG5 and physical mixture of PLGA and PAMAM. (B) Graph showing the size of PMG5 measured overtime at 37° C. and room temperature (RT, 22.5° C.). (C) Graph showing the size distribution of PMG5 measured overtime at 37° C. and room temperature (RT, 22.5° C.). (D) Graph showing the net surface charge of PMG5 measured overtime at 37° C. and room temperature (RT, 22.5° C.). Data are plotted as mean±SD.

FIGS. 6A-6B. Evaluating the complexation of CpG B to PMG5. (A) Graph showing the zetapotential difference as CpG B quantity decreases (positive zetapontential values indicate complete complexion with CpB B). (B) Gel electrophoresis (80 mAmps 150 Volts for 30 minutes), all quantities of CpG B were complexed with 1 mg weighed nanoparticles (0.4523 mg PMG5 was added to the well). Lanes represent: (1) 1 kb ladder 10,000 base pairs (Bp) (top) to 250 Bp (bottom); (2) PMG5; (3) 0.5 mg free CpG B; (4) PMG5 with 0.5 mg CpG B; (5) PMG5 with 0.25 mg CpG B: (6) 377 PMG5 with 0.125 mg CpG B; (7) PMG5 with 0.0625 mg CpG B; (8) PMG5 with 0.03125 mg CpG B; (9) PMG5 with 0.015625 mg CpG B; (10) PMG5 with 0.0078 mg CpG B. Data are plotted as mean±SD.

FIGS. 7A-7B. Evaluation of the cell viability using MTS assay. (A) Graph showing the effect of PMG5 on cell viability of B16.F10. (B) Graph showing effect of PMG5 on cell viability of BMDCs. Data are plotted as mean 384±SD.

FIGS. 8A-8F. In vitro stimulation of bone marrow derived dendritic cells (BMDCs) incubated with different experimental groups. (A) Graph showing difference in CD80 expression. (B) Graph showing difference in MHC-I expression. (C) Graph showing difference in CD40 expression. (D) Graph showing difference in IL-6 secretion. (E) Graph showing difference in IP-10 secretion. (F) Graph showing difference in Th1/Th2 bias. Data are plotted as mean±SD.

FIGS. 9A-9L. Anti-tumor efficacy of PMG5 particles with Ad5-TRP2. (A-H) Tumor volume curves of mice treated with designated treatment. (I) Table showing the median survival of mice. (J) Survival curve of mice treated with different treatments after being challenged with tumors. (K) Graph showing the percent of CD8+ TRP2+ T cells in the peripheral blood (14 days after tumor challenge). (L) Graph showing the average weight of mice overtime. Data are plotted as mean±SD.

FIGS. 10A-10F. Characterization of PMG3 and PMG4. (A) Graph comparing the size of PMG3 before and after lyophilization. (B) Graph comparing the size distribution of PMG3 before and after lyophilization. (C) Graph comparing the net surface charge of PMG4 before and after lyophilization. (D) Graph comparing the size of PMG4 before and after lyophilization. (E) Graph comparing the size distribution of PMG4 before and after lyophilization. (F) Graph comparing the net surface charge of PMG4 before and after lyophilization. Data are plotted as mean±SD.

FIGS. 11A-11F. Stability of PMG3, PMG4 and PMG5 in nanopure water at two different temperatures 37° C. and room temperature (RT, 22.5° C.). (A) Graph showing the size measurements of PMG3. (B) Graph showing the size distribution measurements of PMG3. (C) Graph showing the net surface charge measurements of PMG3. (D) Graph showing the size measurements of PMG4. (E) Graph showing the size distribution measurements of PMG4. (F) Graph showing the net surface charge measurements of PMG4. Data are plotted as mean±SD.

FIGS. 12A-12D. Evaluating the cytotoxic effects of PMG3 PMG4 on BMDCs (primary murine cells) and B16.F10 cells (murine melanoma). (A) Graph showing the effect of PMG3 on cell viability of BMDCs. (B) Graph showing the effect of PMG4 on cell viability of BMDCs. (C) Graph showing the effect of PMG3 on cell viability of B16F10. (D) Graph showing the effect of PMG4 on cell viability of B16.F10. Data are plotted as mean±SD.

FIGS. 13A-13K. Anti-tumor efficacy of PM particles with Ad5-TRP2. (A-H) Tumor volume curves of mice treated with different designated treatment. (1) Graph showing the percent of CD8+ TRP2+ T cells in the peripheral blood (14 days after tumor challenge). (J) Survival curve of mice treated with different treatments after being challenged with tumors. (K) Graph showing the average weight of mice over time. Data are plotted as mean±SD.

FIGS. 14A-14G. Preparation and Characterization of PMG5. (A) Diagram showing the different stages of the synthesis of PMG5. (B and C) TEM images of PMG5 at different magnifications (negative stain, 1.5% phosphotungstic acid). (D) Scanning electron photomicrograph of PMG5 (scale bar=3 μm while the scale bar on inset image=100 nm). (E) Graph showing the hydrodynamic diameter of indicated groups. (F) Graph showing the size distribution (polydispersity index, PDI) of various groups. (G) Graph showing the net surface charge (zeta-potential) of indicated groups. Data are plotted as mean±SD. Error bars represent standard deviation, n=9.

FIGS. 15A-15G. Evaluation of the three PLGA/PAMAM-based formulations with three different particle sizes. (A) Graph showing the size of the particles. (B) Graph showing the size distribution) of particles. (C) Graph showing the net surface charge (zeta-potential) of particles. (D) Graph showing the uptake of the three formulations by BMDC after 48 hours of incubation in vitro. (E) Graph showing the effect of lyophilization conditions on the uptake of PMG5 by BMDCs after 48 hours of incubation in vitro. (F) Graph showing the dose-uptake relationship of PMG5 by BMDCs after 48 hours of incubation in vivo. (G) Graph showing the effects of various inhibitors on the uptake of PMG5 by BMDCs after 3 hours of incubation in vitro. Data are plotted as mean±SD. Error bars represent standard deviation, n=9. Probability was determined by one-way ANOVA with Tukey post-test. Differences in the scale of MFI between graphs F and G are due to the differences in incubation times (48 hours vs 3 hours, respectively). PDI—polydispersity index; MFI—mean fluorescence intensity; LYO—lyophilization. Large and medium-sized particles were micron-sized particles and submicron-sized particles, respectively, that were also made from PLGA and PAMAM in the same ratios described in the section.

FIGS. 16A-16F. In vitro stimulation of BMDCs. BMDCs were incubated for 2 days with indicated treatment and then cells and supernatants were harvested for analysis of surface marker expression and cytokine secretion, respectively. (A) Graph showing relative CD80 expression. (B) Graph showing relative MHC class I expression. (C) Graph showing relative CD40 expression. (D) Graph showing IL-6 secretion levels. (E) Graph showing difference in IP-10 secretion. (F) Graph showing difference in Th1/Th2 bias. Data are plotted as mean±SD. Probability values are determined by one-way ANOVA with Tukey post-test.

FIGS. 17A-17B. Prophylactic immunization of C57BL/6J mice with varying doses of Ad5-TRP2. Mice (n=5/group) were vaccinated SC with the indicated dose of Ad5-TRP2 and then (A) assessed for levels of TRP2-specific CD3+CD8+T lymphocytes (on day 14 post vaccination) using direct immunostaining (see methods section) and flow cytometry (expressed as a percentage of total CD3+CD8+T lymphocytes). Error bars represent standard deviation. *=p<0.05, ***=p<0.001 as determined by one-way ANOVA with Tukey post-test. On day 14 post vaccination all mice were challenged with B16.F10 cells and (B) survival was recorded and a survival graph generated using Prism software. *=p<0.05 (for all treated groups compared to naïve group) as determined by Log-rank test adjusted for multiple comparisons.

FIGS. 18A-18J. Therapeutic Immunizations of C57BL/6J mice with Ad5-TRP2 and peritumoral administrations of PMG5. (A-F) Tumor volume curves of mice treated with designated treatment. (G) Table showing the median survival of mice. (1) Survival curve of mice treated with different treatments after being challenged with tumors. (H) Graph showing the percent of CD8+ TRP2+ T cells in the peripheral blood (14 days after tumor challenge). (J) Graph showing the average weight of mice overtime. Data are plotted as mean±SD. For (H) probability determined by one-way ANOVA with Tukey post-test. For (I) probability determined by Log Rank test with all groups compared to the Naïve group, adjusted for multiple comparisons.

FIGS. 19A-19H. Therapeutic Immunizations of C57BL/6J mice with Ad5-TRP2 and peritumoral administrations of PMG3, PMG4, or PMG5. (A-E) Tumor volume curves of mice treated with indicated treatments. (F) Graph showing the percent of TRP2-specific CD8+ T cells in the peripheral blood (14 days after tumor challenge). (G) Survival curve of mice treated with different treatments after being challenged with tumors. Numbers in brackets indicated the number (numerator) still alive at day 100. (H) Table showing the median survival of mice. Data are plotted as mean±SD. Probability as determined by one-way ANOVA with Tukey post-test.

FIGS. 20A-20H. Effect of therapeutic immunizations of C57BL/6J mice with Ad5-TRP2 combined with systemic immune checkpoint modulation and peritumoral administrations of PMG3, PMG4, or PMG5. (A-E) Tumor volume curves of mice treated with different designated treatments. (F) Graph showing the percent of TRP2-specific CD8+ T cells in the peripheral blood (14 days after tumor challenge). (G) Survival curve of mice treated with different treatments after being challenged with tumors. (H) Table showing the median survival of mice. Data are plotted as mean±SD. The numbers above the graphs refer to the probability as determined by one-way ANOVA with Tukey's post-test.

FIGS. 21A-21F. Evaluation of the effector immune cell population responsible for antitumor efficacy. (A) survival curve of mice treated with Ad5-TRP2 PMG5±depletion of indicated immune cell populations. (B) Survival curve of mice treated with Ad5-TRP2/PMG5 ICM±depletion of indicated immune cell populations. (C) Graph showing the average weight of mice treated with Ad5-TRP2 PMG5±depletion of indicated immune cell populations over time. D. Graph showing the average weight of mice treated with Ad5-TRP2/PMG5 ICM±depletion of indicated immune cell populations overtime. (E-F). Table showing the median survival of mice. Data are plotted as mean±SD. Statistical significance was determined using the Log-Rank test adjusted for multiple comparisons. N=5 mice per group.

FIGS. 22A-22D. Thermal Stability of PMG5. (A) Differential scanning calorimeter (DSC) thermogram of PMG5 and physical mixture of PLGA and PAMAM. (B) Graph showing the size of PMG5 measured overtime at 37° C. and room temperature (RT, 22.5° C.). (C) Graph showing the size distribution of PMG5 measured over time at 37° C. and RT. (D) Graph showing the net surface charge of PMG5 measured overtime at 37° C. and RT. Data are plotted as mean±SD.

FIG. 23. Levels of TRP2-specific CD8+T lymphocytes in PBLs from mice following therapeutic vaccination with Ad5-TRP2 at 1×10⁸ versus 5×10⁸ PFU. Mice were challenged with B16.F10 cells (Day 0) followed by Ad5-TRP2 (1×10⁸ PFU) or Ad5-TRP2 (5×10⁸ PFU) vaccination (Day 1 PTC). N=10/group for Ad5-TRP2 (1×10⁸ PFU) and n=4/group for Ad5-TRP2 (5×10⁸ PFU). Levels of TRP2-specific CD8+T lymphocytes (expressed as a percentage of total CD3+CD8+T lymphocytes) 14 days post-vaccination in PBLs of mice are shown. Statistical analysis was performed using an unpaired t-test (two-tailed) revealing no significant difference.

FIGS. 24A-24D. Characterization of PMG3, PMG4, PMG5. (A) Graph comparing the hydrodynamic diameter of different PM formulations. (B) Graph comparing the net surface charge of different PM formulations. (C) Graph comparing the size distribution of PM formulations. (D) Data are plotted as mean±SD. ****=p<0.0001, ***=p<0.001, **=p<0.01. Table comparing the particle characteristics of PMG3, PMG4, PMG5.

FIGS. 25A-25F. Stability of PMG3, PMG4, and PMG5 in Nanopure water at two different temperatures: 37° C. and room temperature (RT, 22.5° C.). (A) Graph showing the size measurements of PMG3 over time. (B) Graph showing the size distribution measurements of PMG3 over time. (C) Graph showing the net surface charge measurements of PMG3 overtime. (D) Graph showing the size measurements of PMG4 overtime. (E) Graph showing the size distribution measurements of PMG4 over time. (F) Graph showing the net surface charge measurements of PMG4 over time. Data are plotted as mean±SD.

FIGS. 26A-26F. Evaluating the cytotoxic effects of PMG3 PMG4 and PMG5 on BMDCs (primary murine cells) and B16.F10 cells (murine melanoma). Graphs showing the effect of (A) PMG3 (B) PMG4 and (C) PMG5 on the cell viability of BMDCs. Graphs showing the effect of (D) PMG3 (E) PMG4 and (F) PMG5 on the cell viability of B16.F10 cells. Data are plotted as mean±SD. *=p<0.05, ***=p<0.001, ****=p<0.0001, probability determined by one-way ANOVA with Tukey post-test.

FIGS. 27A-27B. Levels of TRP2-specific CD8+T lymphocytes in PBLs following Ad5-TRP2/immune checkpoint therapy. Mice were challenged with B16.F10 cells (Day 0) followed by Ad5-TRP2 or Ad5-PSA vaccination (Day 1) and then α4-1BB and/or αPD1 (Days 8, 10, 13, 16, and 19). N=10 per group except where mice were required to be sacrificed due to tumor volume reaching end-point criteria prior to day 14 post-vaccination; or where outliers were removed (see below) (a) Levels of TRP2-specific CD8+T lymphocytes 14 days post-vaccination in PBLs of mice treated with indicated combinations. The numbers above bar refer to the probability as determined by one-way ANOVA with Tukey post-test. b) Levels of TRP2-specific T cells in PBLs of mice treated with AdTRP2 (1×10⁸ PFU/mouse)+α4-1BB/PD1 (n=10) versus AdTRP2 (5×10⁸ PFU/mouse)+α4-1BB/PD1 (n=7). Statistical analysis was performed using an unpaired t-test (two-tailed). **(p=0.0035).

FIG. 28. Tumor volumes of B16.F10-challenged mice and effect of Ad5-TRP2 and/or immune checkpoint therapy. Mice were challenged with B16.F10 cells (Day 0) followed by indicated treatment (further details described in methods section). % TF=% mice that were tumor-free at the completion of the study (Day 80). N=10 per group except for the following groups: Ad5-PSA (n=8), Ad5-PSA+αPD1 (n=8), Ad5-TRP2+4-1BB (n=8), Ad5-TRP2 5-fold (n=4), Ad5-TRP2 5-fold+αPD1/4-1BB (n=7).

FIGS. 29A-29B. Survival curve for B16.F10-challenged mice and effect of Ad5-TRP2 and/or immune checkpoint therapy. C57/B16 mice were challenged with B16.F10 cells (Day 0) followed by Ad5-TRP2 or Ad5-PSA vaccination (Day 1) and then anti-4-1BB (α4-1BB) and/or anti-PD1 (αPD1) (Days 8, 10, 13, 16 and 18) (as described in methods). Statistical significance was determined using the Log-Rank test and then the threshold for p was adjusted for multiple comparisons (K=21). ***^{a}=significantly different from naïve and Ad5-PSA groups (p<0.002); *^{b}=significantly different from Ad5-PSA+α4-1BB/PD1 group (p=0.033); ***^{c}=significantly different from Ad5-PSA+α4-1BB/PD1 group (p<0.002).

FIG. 30. Graph showing the average weight of mice over time. Data are plotted as mean±SD.

FIGS. 31A-31K. Evaluation of the effector immune cell population responsible for antitumor efficacy. (A-K) Tumor volume curves of mice treated with different designated treatments.

FIG. 32. Graph showing the percent of CD8+ TRP2+ T cells in the peripheral blood (14 days after vaccination in non-tumor bearing mice). C57/B16 mice (n=3) were vaccinated with Ad5-TRP2 on the left dorsal flank (Day 0), on days 7, 9, 12 mice were given either PBS, PMG3, PMG4, PMG5 on the right dorsal flank subcutaneously and on day 14 were submandibularly bled and the presence of TRP2-specific CD8+T lymphocytes evaluated. Data are plotted as mean±SD.

FIG. 33. Graph showing the uptake of PM formulations by BMDCs. BMDCs were incubated with 0.24 mg of different PM formulations for 48 hours. Statistical analysis was performed using one-way ANOVA with Tukey's multiple comparisons test. Error bars represent the standard deviation. The numbers above the graphs refer to the probability as determined by one-way ANOVA with Tukey's post-test. BMDCs were initially seeded in a 12-well plate in media-containing serum for 24 hours then incubated with PMG3, PMG4, PMG5 for 48 hours. BMDCs were then collected and analyzed using a FACScan flow cytometer.

### DETAILED DESCRIPTION

Cancer vaccines possess the inherent ability to boost the number of tumor-specific T cells. However, at the clinical stage, the efficacy of cancer vaccines as a monotherapy has fallen behind other cancer immunotherapeutic treatment options. Adjuvants formulated on the nanoscale possess the ability to increase the efficacy of cancer vaccines by stimulating the immune system and remodeling the immunosuppressive nature of the tumor microenvironment. To this end, a cationic polymeric nanoparticle formulation was synthesized using PAMAM and PLGA that possesses intrinsic adjuvant properties in vivo. Tumor-challenged mice that were treated with this nanoparticulate adjuvant and vaccinated with an adenovirus-based cancer vaccine showed a significant increase in antigen-specific T cells in the peripheral blood, reduced tumor burden, protection against tumor re-challenge, and a 300% increase in median survival. Combining this approach of local nanoparticulate adjuvant administration with systemic administration of immune checkpoint modulation therapy significantly increased the efficacy of the adenovirus cancer vaccine.

It has been previously demonstrated that combining the Ad5 cancer vaccine (carrying a model TSA) with intratumoral administration of adjuvants such as the pathogen-associated molecular pattern, cytosine guanine oligonucleotides (CpG ODNs), significantly reduces tumor growth and increases survival in mice, along with increasing the proportion of antigen-specific CD8+ T cells in the TME and peripheral blood (Lou et al., 2011; Geary et al., 2011).

Adjuvants formulated as nanoparticles may be particularly useful to aid in overwhelming the immuno-challenges presented by the TME. First, nanoparticles (NPs) of less than 200 nm in diameter have been found to independently traffic to the lymph node without cellular uptake and interact with resident DCs there (Sagiv-Barfi et al., 2018). Adjuvant-loaded NPs that drain independently to the tumor-draining lymph nodes (TDLN) and stimulate resident DCs may also prevent the recruitment of immunosuppressive cell populations (myeloid-derived suppressor cells) (Thomas et al., 2014).

To this end, formulating a NP-based adjuvant system provides a possible route to further enhance the immunogenicity of vaccines, e.g., an Ad5-based cancer vaccine. Nanoscopic compounds such as dendrimers have opened new avenues in the development of delivery systems. Since their debut in 1984, polyamidoamine (PAMAM) dendrimers have gained the attention of many researchers as a tool for gene delivery (Santos et al., 2010; Svenson & Tomalia, 2005; Dufes et al., 2005) and drug delivery (Chauhan & Kaul, 2018; Biswas et al., 2013) due to its abundance of surface amines, wide possibility for drug/gene association and lack of immunogenicity (Bono et al., 2019). Despite its great potential and biomedical applications, PAMAM is known to be toxic; however, this has been surmounted by chemically modifying its surface to shield the highly cationic surface (Bono et al., 2019; Labieniec-Watala & Watala, 2015; Araugo et al., 2018). This of course limits the very attributes which differentiated PAMAM dendrimers to begin with.

In an effort to develop an Ad5-based cancer vaccine while utilizing adjuvants formulated as NPs, a PAMAM dendrimer-based NP formulation was developed utilizing a solution of PLGA and PAMAM to form a NP with the attributes of both polymers. This NP is different from previous works where PAMAM was physically adsorbed to the surface of PLGA NPs (Intra & Salem, 2010) or the PAMAM was chemically modified to yield beneficial attributes. Herein below, this PLGA/PAMAM NP formulation is termed PM and the resulting bilayer NP, where PAMAM is integrated with a PLGA core which is in contrast to physical adsorption, was prepared from a single polymer solution. The mixture of polymer ratios, e.g., 8:1 to 4:1 PLGA:PAMAM by weight, allows for a stable, immunogenic NP that can be efficiently endocytosed by DCs. For example, in a 8:1 formulation, 125 μL of PAMAM (6.25 mg) is employed and in 4:1 formulation, 250 μL of PAMAM is employed. PLGA may be in a ratio of 50:50, 75:25, 80:20, or 90:10 (lactic acd:glycolic acid).

The exemplary PM NPs described herein were combined with an attenuated serotype 5 adenovirus encoding the melanoma TAA, tyrosinase related protein 2 (Ad5-TRP2). Melanoma represents an excellent model to prove the effectiveness of this formulation for several reasons. Firstly, the NIH Surveillance and Epidemiology and End Results Program (SEER) puts malignant melanoma as the fifth most common diagnosed cancer in the United States with an estimated 96,480 new cases in 2019. While melanoma, if detected early (melanoma in-situ), can be treated with surgery or targeted therapeutic agents based on a patient's mutation status, for advance stage melanoma (stage III or IV), few options are available to patients (Wrobel et al., 2019). Thus, immunotherapy represents a viable alternative for these patients.

As shown herein, combining PM with Ad5-TRP2 resulted in inhibition of tumor growth, increased survival in mice and this is in part due to a greatly increased systemic CD8+ T cell response. Mice given this treatment also showed a protective response from tumor re-challenge. This response was not noted in groups that were given CpG, a potent TLR-9 agonist.

### Exemplary Nanoparticle Formulations

In one embodiment, the formulation comprises nanoparticles comprising a synthetic polymer and a dendrimer. The disclosed particles, e.g., biodegradable nanoparticles, may include or may be formed from biodegradable polymeric molecules which may include, but are not limited to polylactic acid (PLA), polyglycolic acid (PGA), co-polymers of PLA and PGA (i.e., polyactic-co-glycolic acid (PLGA)), poly-ε-caprolactone (PCL), polyethylene glycol (PEG), poly(3-hydroxybutyrate), poly(p-dioxanone), polypropylene fumarate, poly(orthoesters), polyol/diketene acetals addition polymers, poly-alkyl-cyano-acrylates (PAC), poly(sebacic anhydride) (PSA), poly(carboxybiscarboxyphenoxyphenoxy hexone (PCPP) poly[bis (p-carboxypheonoxy)methane](PCPM), copolymers of PSA, PCPP and PCPM, poly(amino acids), poly(pseudo amino acids), polyphosphazenes, derivatives of poly[(dichloro)phosphazenes] and poly[(organo)phosphazenes], poly-hydroxybutyric acid, or S-caproic acid, elastin, or gelatin. (See, e.g., Kumari et al., Colloids and Surfaces B: Biointerfaces 75 (2010) 1-18; and U.S. Pat. Nos. 6,913,767; 6,884,435; 6,565,777; 6,534,092; 6,528,087; 6,379,704; 6,309,589; 6,264,987; 6,210,707; 6,090,925; 6,022,564; 5,981,719; 5,871,747; 5,723,269; 5,603,960; and 5,578,709; and U.S. Published Application No. 2007/0081972; and International Application Publication Nos. WO 2012/115806; and WO 2012/054425; the contents of which are incorporated herein by reference in their entireties).

The disclosed nanoparticles may be prepared by methods known in the art. (See, e.g., Nagavarma et al., Asian J. of Pharma. And Clin. Res., Vol 5, Suppl 3, 2012, pages 16-23; Cismaru et al., Rev. Roum. Chim., 2010, 55(8), 433-442; and International Application Publication Nos. WO 2012/115806; and WO 2012/054425; the contents of which are incorporated herein by reference in their entireties). Suitable methods for preparing particles may include methods that utilize a dispersion of a preformed polymer, which may include but are not limited to solvent evaporation, nanoprecipitation, emulsification/solvent diffusion, salting out, dialysis, and supercritical fluid technology. In some embodiments, the particles may be prepared by forming a double emulsion (e.g., water-in-oil-in-water) and subsequently performing solvent-evaporation. The particles may be subjected to further processing steps such as washing and lyophilization, as desired. Optionally, the particles may be combined with a preservative such as a cryoprotectant (e.g., trehalose).

In one embodiment, the particles have a mean effective diameter of less than 500 nm, e.g., the particles have a mean effective diameter of between about 1 nm and about 500 nm, e.g., between about 50 nm and about 125 nm, about 100 nm and about 200 nm, about 150 nm and about 250 nm, about 100 nm to about 150 nm, or about 150 nm to 225 nm. In one embodiment, the particles have a mean effective diameter of less than 1 micron. The size of the particles (e.g., mean effective diameter) may be assessed by known methods in the art, which may include but are not limited to transmission electron microscopy (TEM), scanning electron microscopy (SEM), Atomic Force Microscopy (AFM), Photon Correlation Spectroscopy (PCS), Nanoparticle Surface Area Monitor (NSAM), Condensation Particle Counter (CPC), Differential Mobility Analyzer (DMA), Scanning Mobility Particle Sizer (SMPS), Nanoparticle Tracking Analysis (NTA), X-Ray Diffraction (XRD), Aerosol Time of Flight Mass Spectroscopy (ATFMS), and Aerosol Particle Mass Analyzer (APM).

In one embodiment, a particles comprise polymers including but not limited to poly(lactic-co-glycolic acid) (PLGA), polylactic acid (PLA), linear and/or branched PEI with differing molecular weights (e.g., 2, 22 and 25 kDa), dendrimers such as polyamidoamine (PAMAM) and polymethoacrylates; lipids including but not limited to liposomes, emulsions, DOTAP, DOTMA, DMRIE, DOSPA, distearoylphosphatidylcholine (DSPC), DOPE, or DC-cholesterol; peptide based vectors including but not limited to poly-L-lysine or protamine; or poly(β-amino ester), chitosan, PEI-polyethylene glycol, PEI-mannose-dextrose, DOTAP-cholesterol or RNAiMAX.

In one embodiment, the particle is a glycopolymer-based particle, poly(glycoamidoamine)s (PGAAs). These materials are created by polymerizing the methylester or lactone derivatives of various carbohydrates (D-glucarate (D), meso-galactarate (G), D-mannarate (M), and L-tartarate (T)) with a series of oligoethyleneamine monomers (containing between 1-4 ethylenamines (Liu and Reineke, 2006). A subset composed of these carbohydrates and four ethyleneamines in the polymer repeat units may yield exceptional delivery efficiency.

In one embodiment, the particles comprise polyethyleneimine (PEI), polyamidoamine (PAMAM), or polyamidoamine (PAMAM) dendrimers. Polyamidoamine dendrimers suitable for preparing the particles may include 3rd-, 4th-, 5th-, or at least 6th-generation dendrimers.

In one embodiment, the delivery vehicle may be particles or liposomes comprising a cationic lipid, e.g., N-[1-(2,3-dioleoyloxy)propel]-N,N,N-trimethylammonium (DOTMA), 2,3-dioleyloxy-N-[2-spermine carboxamide] ethyl-N,N-dimethyl-1-propanammonium trifluoracetate (DOSPA, Lipofectamine); 1,2-dioleoyl-3-trimethylammonium-propane (DOTAP); N-[1-(2,3-dimyristloxy) propyl]; N,N-dimethyl-N-(2-hydroxyethyl) ammonium bromide (DMRIE), 3-p-[N—(N,N′-dimethylaminoethane) carbamoyl] cholesterol (DC-Chol); dioctadecyl amidoglyceryl spermine (DOGS, Transfectam); or imethyldioctadeclyammonium bromide (DDAB). The positively charged hydrophilic head group of cationic lipids usually consists of monoamine such as tertiary and quaternary amines, polyamine, amidinium, or guanidinium group. A series of pyridinium lipids have been developed (Zhu et al., 2008; van der Woude et al., 1997; Ilies et al., 2004). In addition to pyridinium cationic lipids, other types of heterocyclic head groups include imidazole, piperizine and amino acid. The main function of cationic head groups is to condense negatively charged molecules by means of electrostatic interaction to slightly positively charged particles, leading to enhanced cellular uptake and endosomal escape.

Lipids having two linear fatty acid chains, such as DOTMA, DOTAP and SAINT-2, or DODAC, may be employed as a delivery vehicle, as well as tetraalkyl lipid chain surfactant, the dimer of N,N-dioleyl-N,N-dimethylammonium chloride (DODAC). All the trans-orientated lipids regardless of their hydrophobic chain lengths (C_{18.1}, C_{18.1} and C_{20.1}) appear to enhance the transfection efficiency compared with their cis-orientated counterparts.

The structures of polymers include but are not limited to linear polymers such as chitosan and linear poly(ethyleneimine), branched polymers such as branch poly(ethyleneimine) (PEI), circle-like polymers such as cyclodextrin, network (crosslinked) type polymers such as crosslinked poly(amino acid) (PAA), and dendrimers. Dendrimers consist of a central core molecule, from which several highly branched arms ‘grow’ to form a tree-like structure with a manner of symmetry or asymmetry. Examples of dendrimers include polyamidoamine (PAMAM) and polypropylenimine (PPI) dendrimers.

DOPE and cholesterol are commonly used neutral co-lipids for preparing liposomes. Branched PEI-cholesterol water-soluble lipopolymer conjugates self-assemble into cationic micelles. Pluronic (poloxamer), a non-ionic polymer and SP1017, which is the combination of Pluronics L61 and F127, may also be used.

In one embodiment, PLGA particles are employed to increase the encapsulation frequency although other materials, for example, PEI, DOTMA, DC-Chol, or CTAB, may be used.

In one embodiment, the particles comprise hydrogels of poloxamers, polyacrylamide, poly(2-hydroxyethyl methacrylate), carboxyvinyl-polymers (e.g., Carbopol 934, Goodrich Chemical Co.), cellulose derivatives, e.g., methylcellulose, cellulose acetate and hydroxypropyl cellulose, polyvinyl pyrrolidone or polyvinyl alcohols, or combinations thereof.

In some embodiments, a biocompatible polymeric material is derived from a biodegradable polymeric such as collagen, e.g., hydroxylated collagen, fibrin, polylactic-polyglycolic acid, or a polyanhydride. Other examples include, without limitation, any biocompatible polymer, whether hydrophilic, hydrophobic, or amphiphilic, such as ethylene vinyl acetate copolymer (EVA), polymethyl methacrylate, polyamides, polycarbonates, polyesters, polyethylene, polypropylenes, polystyrenes, polyvinyl chloride, polytetrafluoroethylene, N-isopropylacrylamide copolymers, poly(ethylene oxide)/poly(propylene oxide) block copolymers, poly(ethylene glycol)/poly(D,L-lactide-co-glycolide) block copolymers, polyglycolide, polylactides (PLLA or PDLA), poly(caprolactone) (PCL), or poly(dioxanone) (PPS).

In another embodiment, the biocompatible material includes polyethyleneterephalate, polytetrafluoroethylene, copolymer of polyethylene oxide and polypropylene oxide, a combination of polyglycolic acid and polyhydroxyalkanoate, gelatin, alginate, poly-3-hydroxybutyrate, poly-4-hydroxybutyrate, and polyhydroxyoctanoate, and polyacrylonitrilepolyvinylchlorides.

In one embodiment, the following polymers may be employed, e.g., natural polymers such as starch, chitin, glycosaminoglycans, e.g., hyaluronic acid, dermatan sulfate and chrondrotin sulfate, and microbial polyesters, e.g., hydroxyalkanoates such as hydroxyvalerate and hydroxybutyrate copolymers, and synthetic polymers, e.g., poly(orthoesters) and polyanhydrides, and including homo and copolymers of glycolide and lactides (e.g., poly(L-lactide, poly(L-lactide-co-D,L-lactide), poly(L-lactide-co-glycolide, polyglycolide and poly(D,L-lactide), pol(D,L-lactide-coglycolide), poly(lactic acid colysine) and polycaprolactone.

In one embodiment, the biocompatible material is derived from isolated extracellular matrix (ECM). ECM may be isolated from endothelial layers of various cell populations, tissues and/or organs, e.g., any organ or tissue source including the dermis of the skin, liver, alimentary, respiratory, intestinal, urinary or genital tracks of a warm blooded vertebrate. ECM may be from a combination of sources. Isolated ECM may be prepared as a sheet, in particulate form, gel form and the like.

The biocompatible polymer may comprise silk, elastin, chitin, chitosan, poly(d-hydroxy acid), polyanhydrides, or poly(orthoesters). More particularly, the biocompatible polymer may be formed polyethylene glycol, poly(lactic acid), poly(glycolic acid), copolymers of lactic and glycolic acid, copolymers of lactic and glycolic acid with polyethylene glycol, poly(E-caprolactone), poly(3-hydroxybutyrate), poly(p-dioxanone), polypropylene fumarate, poly(orthoesters), polyol/diketene acetals addition polymers, poly(sebacic anhydride) (PSA), poly(carboxybiscarboxyphenoxyphenoxy hexone (PCPP) poly[bis (p-carboxypheonoxy) methane] (PCPM), copolymers of SA, CPP and CPM, poly(amino acids), poly(pseudo amino acids), polyphosphazenes, derivatives of poly[(dichloro)phosphazenes] or poly[(organo) phosphazenes], poly-hydroxybutyric acid, or S-caproic acid, polylactide-co-glycolide, polylactic acid, polyethylene glycol, cellulose, oxidized cellulose, alginate, gelatin or derivatives thereof.

Thus, the polymer may be formed of any of a wide range materials including polymers, including naturally occurring polymers, synthetic polymers, or a combination thereof. In one embodiment, the scaffold comprises biodegradable polymers. In one embodiment, a naturally occurring biodegradable polymer may be modified to provide for a synthetic biodegradable polymer derived from the naturally occurring polymer. In one embodiment, the polymer is a poly(lactic acid) (“PLA”) or poly(lactic-co-glycolic acid) (“PLGA”). In one embodiment, the scaffold polymer includes but is not limited to alginate, chitosan, poly(2-hydroxyethylmethacrylate), xyloglucan, co-polymers of 2-methacryloyloxyethyl phosphorylcholine, poly(vinyl alcohol), silicone, hydrophobic polyesters and hydrophilic polyester, poly(lactide-co-glycolide), N-isoproylacrylamide copolymers, poly(ethylene oxide)/poly(propylene oxide), polylactic acid, poly(orthoesters), polyanhydrides, polyurethanes, copolymers of 2-hydroxyethylmethacrylate and sodium methacrylate, phosphorylcholine, cyclodextrins, polysulfone and polyvinylpyrrolidine, starch, poly-D,L-lactic acid-para-dioxanone-polyethylene glycol block copolymer, polypropylene, poly(ethylene terephthalate), poly(tetrafluoroethylene), poly-epsilon-caprolactone, or crosslinked chitosan hydrogels.

### Pharmaceutical Nanoparticle Containing Compositions

The disclosure provides a composition comprising, consisting essentially of, or consisting of nanoparticles a synthetic polymer and a dendrimer and optionally a pharmaceutically acceptable (e.g., physiologically acceptable) carrier. In one embodiment, additional components can be included that do not materially affect the composition (e.g., adjuvants, buffers, stabilizers, anti-inflammatory agents, solubilizers, preservatives, etc.). In one embodiment, when the composition consists of the nanoparticles comprising a synthetic polymer and a dendrimer and optionally the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition may be sterile. The composition can be frozen or lyophilized for storage and reconstituted in a suitable sterile carrier prior to use. The compositions can be generated in accordance with conventional techniques described in, e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the nanoparticles are administered in a composition formulated to protect the nanoparticles from damage prior to administration. In addition, one of ordinary skill in the art will appreciate that the nanoparticles can be present in a composition with other biologically-active agents.

Injectable depot forms are envisioned including those having biodegradable polymers such as polylactide-polyglycolide. Depending on the ratio of drug to polymer, and the nature of the particular polymer employed, the rate of drug release can be controlled. Examples of other biodegradable polymers include poly(orthoesters) and polyanhydrides. Depot injectable formulations are also prepared by entrapping the drug optionally in a complex with a polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the nanoparticles in the composition administered to the mammal will depend on a number of factors, including the size (mass) of the mammal, the extent of any side-effects, the particular route of administration, and the like. In one embodiment, the method comprises administering a “therapeutically effective amount” of the composition. A “therapeutically effective amount” refers to an amount effective, at dosages and for periods of time necessary, to achieve a desired therapeutic result, e.g., enhancing an immune response. The therapeutically effective amount may vary according to factors such as the extent of the disease or disorder, age, sex, and weight of the individual, and the ability of the nanoparticles to elicit a desired response in the individual. One of ordinary skill in the art can readily determine an appropriate dose range to provide for enhancing an immune response, e.g., an adaptive immune response, in a patient having a particular disease or disorder, or in need of eliciting an adaptive immune response, based on these and other factors that are well known in the art.

In one embodiment, the composition is administered once to the mammal. It is believed that a single administration of the composition may be beneficial. However, in certain cases, it may be appropriate to administer the composition multiple times during a period. For example, the composition may be administered to the mammal two or more times (e.g., 2, 3, 4, 5, 6, 7, 8, 9, or 10 or more times) during a period.

The present disclosure provides pharmaceutically acceptable compositions which comprise an amount of the nanoparticles as described above.

### Routes of Administration, Dosages and Dosage Forms

Administration of the nanoparticles may be continuous or intermittent, depending, for example, upon the recipients physiological condition, and other factors known to skilled practitioners. The administration of the nanoparticles may be essentially continuous over a preselected period of time, may be in a series of spaced doses, or may be a single dose. Both local administration, e.g., intratumoral, peritumoral or intrathecal, and systemic administration, e.g., intravenous, are envisioned. In one embodiment, compositions may be subcutaneously, intramuscularly, intradermally, or intravascularly delivered.

One or more suitable unit dosage forms comprising the nanoparticles, which may optionally be formulated for sustained release, can be administered by a variety of routes including local, e.g., intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the nanoparticles with liquid carriers, solid matrices, semi-solid carriers, finely divided solid carriers or combinations thereof, and then, if necessary, introducing or shaping the product into the desired delivery system.

The amount of the nanoparticles administered to achieve a particular outcome will vary depending on various factors including, but not limited to the condition, patient specific parameters, e.g., height, weight and age, and whether prevention or treatment, is to be achieved.

The nanoparticles may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises, e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

The nanoparticles may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride. pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The nanoparticles can be provided in a dosage form containing an amount effective in one or multiple doses. The nanoparticles may be administered in dosages of at least about 0.0001 mg/kg to about 1 mg/kg, of at least about 0.001 mg/kg to about 0.5 mg/kg, at least about 0.01 mg/kg to about 0.25 mg/kg, at least about 0.01 mg/kg to about 0.25 mg/kg of body weight, at least about 0.1 mg/kg to about 25 mg/kg of body weight, at least about 1 mg/kg to about 250 mg/kg of body weight, at least about 10 mg/kg to about 500 mg/kg of body weight, at least about 0.1 g/kg to about 0.5 g/kg of body weight, or at least about 0.5 g/kg to about 2 g/kg of body weight, although other dosages may provide beneficial results. The amount administered will vary depending on various factors including, but not limited to, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. In one embodiment, from 0.0001 to 1 mg or more, e.g., up to 1 g, in individual or divided doses, e.g., from 0.001 to 0.5 mg, or 0.01 to 0.1 mg, of nanoparticles can be administered.

Pharmaceutical formulations containing the nanoparticles can be prepared by procedures known in the art using well known and readily available ingredients. For example, the agent can be formulated with common excipients, diluents, or carriers, and formed into tablets, capsules, suspensions, powders, and the like. The nanoparticles can also be formulated as elixirs or solutions appropriate for parenteral administration, for instance, by intramuscular, subcutaneous or intravenous routes.

The pharmaceutical formulations can also take the form of an aqueous or anhydrous solution, e.g., a lyophilized formulation, or dispersion, or alternatively the form of an emulsion or suspension.

In one embodiment, the nanoparticles may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the prior art. It is possible, for example, to prepare solutions using one or more organic solvent(s) that is/are acceptable from the physiological standpoint.

For administration to the upper (nasal) or lower respiratory tract by inhalation, the nanoparticle composition is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the nanoparticles and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the nanoparticle composition may be administered via nose drops, a liquid spray, such as via a plastic bottle atomizer or metered-dose inhaler. Typical atomizers are the Mistometer (Wintrop) and the Medihaler (Riker).

The local delivery of the nanoparticle composition can also be by a variety of techniques which administer the nanoparticle composition at or near the site of disease, e.g., using a catheter or needle. Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Delivery Vectors

Immunogen (antigen) delivery vectors include, for example, viral vectors, liposomes and other lipid-containing complexes, such as lipoplexes (DNA and cationic lipids), polyplexes, e.g., DNA complexed with cationic polymers such as polyethylene glycol, nanoparticles, e.g., magnetic inorganic nanoparticles that bind or are functionalized to bind DNA such as Fe₃O₄ or MnO₂ nanoparticles, microparticles, e.g., formed of polylactide polygalactide reagents, nanotubes, e.g., silica nanotubes, and other macromolecular complexes capable of mediating delivery of a gene to a host cell. Vectors can also comprise other components or functionalities that further modulate gene delivery and/or gene expression, or that otherwise provide beneficial properties to the cells. Such other components include, for example, components that influence binding or targeting to cells (including components that mediate cell-type or tissue-specific binding); components that influence uptake of the vector by the cell; components that influence localization of the transferred gene within the cell after uptake (such as agents mediating nuclear localization); and components that influence expression of the gene. Such components also might include markers, such as detectable and/or selectable markers that can be used to detect or select for cells that have taken up and are expressing the nucleic acid delivered by the vector. A large variety of such vectors are known in the art and are generally available.

Exemplary delivery vectors within the scope of the invention include, but are not limited to, viral vectors, e.g., recombinant adenovirus, retrovirus, lentivirus, herpesvirus, poxvirus, papilloma virus, or adeno-associated virus, including viral and non-viral vectors which are present in liposomes, e.g., neutral or cationic liposomes, such as DOSPA/DOPE, DOGS/DOPE or DMRIE/DOPE liposomes, and/or associated with other molecules such as DNA-anti-DNA antibody-cationic lipid (DOTMA/DOPE) complexes, or isolated DNA, e.g., a plasmid. Exemplary viral gene delivery vectors are described below. Immunogen delivery vectors may be administered via any route including, but not limited to, intracranial, intrathecal, intramuscular, buccal, rectal, intravenous or intracoronary administration, and transfer to cells may be enhanced using electroporation and/or iontophoresis, and/or scaffolding such as extracellular matrix or hydrogels, e.g., a hydrogel patch. In one embodiment, a permeation enhancer is employed to enhance indirect delivery to the CNS. In one embodiment, a permeation enhancer is not mployed to enhance indirect delivery to the CNS.

### Retroviral Vectors

Retroviral vectors exhibit several distinctive features including their ability to stably and precisely integrate into the host genome providing long-term transgene expression. These vectors can be manipulated ex vivo to eliminate infectious gene particles to minimize the risk of systemic infection and patient-to-patient transmission. Pseudotyped retroviral vectors can after host cell tropism.

### Lentiviruses

Lentiviruses are derived from a family of retroviruses that include human immunodeficiency virus and feline immunodeficiency virus. However, unlike retroviruses that only infect dividing cells, lentiviruses can infect both dividing and nondividing cells. For instance, lentiviral vectors based on human immunodeficiency virus genome are capable of efficient transduction of cardiac myocytes in vivo. Although lentiviruses have specific tropisms, pseudotyping the viral envelope with vesicular stomatitis virus yields virus with a broader range (Schnepp et al., Meth. Mol. Med., 69:427 (2002)).

### Adenoviral Vectors

Adenoviral vectors may be rendered replication-incompetent by deleting the early (E1A and E1B) genes responsible for viral gene expression from the genome and are optionally stably maintained in the host cells in an extrachromosomal form. These vectors have the ability to transfect both replicating and nonreplicating cells and, in particular, these vectors have been shown to efficiently infect cardiac myocytes in vivo, e.g., after direction injection or perfusion. Adenoviral vectors have been shown to result in transient expression of therapeutic genes in vivo, peaking at 7 days and lasting approximately 4 weeks. The duration of transgene expression may be improved in systems utilizing neural specific promoters. In addition, adenoviral vectors can be produced at very high titers, allowing efficient gene transfer with small volumes of virus.

### Adeno-Associated Virus Vectors

Recombinant adeno-associated viruses (rAAV) are derived from nonpathogenic parvoviruses, evoke essentially no cellular immune response, and produce transgene expression lasting months in most systems. Moreover, like adenovirus, adeno-associated virus vectors also have the capability to infect replicating and nonreplicating cells and are believed to be nonpathogenic to humans. Moreover, they appear promising for sustained cardiac gene transfer (Hoshijima et al., Nat. Med., 8:864 (2002); Lynch et al., Circ. Res., 80:197 (1997)).

AAV vectors include but are not limited to AAV1, AAV2, AAV5, AAV7, AAV8, AAV9 or AAVrh.10.

### Plasmid DNA Vectors

Plasmid DNA is often referred to as “naked DNA” to indicate the absence of a more elaborate packaging system. Direct injection of plasmid DNA to myocardial cells in vivo has been accomplished. Plasmid-based vectors are relatively nonimmunogenic and nonpathogenic, with the potential to stably integrate in the cellular genome, resulting in long-term gene expression in postmitotic cells in vivo. For example, expression of secreted angiogenesis factors after muscle injection of plasmid DNA, despite relatively low levels of focal transgene expression, has demonstrated significant biologic effects in animal models and appears promising clinically (Isner, Nature, 415:234 (2002)). Furthermore, plasmid DNA is rapidly degraded in the blood stream; therefore, the chance of transgene expression in distant organ systems is negligible. Plasmid DNA may be delivered to cells as part of a macromolecular complex, e.g., a liposome or DNA-protein complex, and delivery may be enhanced using techniques including electroporation.

### Pharmaceutical Compositions Comprising Gene Transfer Vector or Nanoparticles

The disclosure provides a composition comprising, consisting essentially of, or consisting of a gene transfer vector(s) and optionally a pharmaceutically acceptable (e.g., physiologically acceptable) carrier. The disclosure provides a composition comprising, consisting essentially of, or consisting of cationic nanoparticles formed of a synthetic polymer and a dendrimer, and optionally a pharmaceutically acceptable (e.g., physiologically acceptable) carrier. In one embodiment, when the composition consists essentially of a recombinant virus, and optionally a pharmaceutically acceptable carrier, additional components can be included that do not materially affect the composition (e.g., adjuvants, buffers, stabilizers, anti-inflammatory agents, solubilizers, preservatives, etc.). In one embodiment, when the composition consists of the gene transfer vector, e.g., recombinant virus, or the cationic nanoparticles, and optionally the pharmaceutically acceptable carrier, the composition does not comprise any additional components. Any suitable carrier can be used within the context of the invention, and such carriers are well known in the art. The choice of carrier will be determined, in part, by the particular site to which the composition may be administered and the particular method used to administer the composition. The composition can be frozen or lyophilized for storage and reconstituted in a suitable sterile carrier prior to use. The compositions can be generated in accordance with conventional techniques described in. e.g., Remington: The Science and Practice of Pharmacy, 21st Edition, Lippincott Williams & Wilkins, Philadelphia, PA (2001).

Suitable formulations for the composition include aqueous and non-aqueous solutions, isotonic sterile solutions, which can contain anti-oxidants, buffers, and bacteriostats, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid carrier, for example, water, immediately prior to use. Extemporaneous solutions and suspensions can be prepared from sterile powders, granules, and tablets of the kind previously described. In one embodiment, the carrier is a buffered saline solution. In one embodiment, the gene transfer vector is administered in a composition formulated to protect the gene transfer vector from damage prior to administration. For example, the composition can be formulated to reduce loss of the gene transfer vector or nanoparticles on devices used to prepare, store, or administer the gene transfer vector or nanoparticles, such as glassware, syringes, or needles. The composition can be formulated to decrease the light sensitivity and/or temperature sensitivity of the gene transfer vector or nanoparticles, or otherwise increase long term stability. To this end, the composition may comprise a pharmaceutically acceptable liquid carrier, such as, for example, those described above, and a stabilizing agent selected from the group consisting of polysorbate 80, L-arginine, polyvinylpyrrolidone, sucrose, trehalose, and combinations thereof. Use of such a composition may extend the shelf life of the gene transfer vector or nanoparticles, facilitate administration, and increase the efficiency of the method. Formulations for gene transfer vector containing compositions are further described in, for example, Wright et al., Curr. Opin. Drug Discov. Devel., 6(2): 174-178 (2003) and Wright et al., Molecular Therapy, 12:171-178 (2005))

The composition also can be formulated to enhance transduction efficiency. In addition, one of ordinary skill in the art will appreciate that the gene transfer vector can be present in a composition with other therapeutic or biologically-active agents. For example, factors that control inflammation, such as ibuprofen or steroids, can be part of the composition to reduce swelling and inflammation associated with in vivo administration of the gene transfer vector. Immune system stimulators or adjuvants, e.g., interleukins, lipopolysaccharide, and double-stranded RNA. Antibiotics, i.e., microbicides and fungicides, can be present to treat existing infection and/or reduce the risk of future infection, such as infection associated with gene transfer procedures.

Injectable depot forms are made by forming microencapsulated matrices of the vectors in biodegradable polymers such as polylactide-polyglycolide. Depending on the ratio of vector to polymer, and the nature of the particular polymer employed, the rate of vector release can be controlled. Examples of other biodegradable polymers include poly(orthoesters) and polyanhydrides. Depot injectable formulations are also prepared by entrapping the vector optionally in a complex with a cationic polymer in liposomes or microemulsions which are compatible with body tissue.

In certain embodiments, a formulation comprises a biocompatible polymer selected from the group consisting of polyamides, polycarbonates, polyalkylenes, polymers of acrylic and methacrylic esters, polyvinyl polymers, polyglycolides, polysiloxanes, polyurethanes and co-polymers thereof, celluloses, polypropylene, polyethylenes, polystyrene, polymers of lactic acid and glycolic acid, polyanhydrides, poly(ortho)esters, poly(butic acid), poly(valeric acid), poly(lactide-co-caprolactone), polysaccharides, proteins, polyhyaluronic acids, polycyanoacrylates, and blends, mixtures, or copolymers thereof.

The composition can be administered in or on a device that allows controlled or sustained release, such as a sponge, biocompatible meshwork, mechanical reservoir, or mechanical implant. Implants (see, e.g., U.S. Pat. No. 5,443,505), devices (see, e.g., U.S. Pat. No. 4,863,457), such as an implantable device, e.g., a mechanical reservoir or an implant or a device comprised of a polymeric composition, are particularly useful for administration of the inventive gene transfer vector. The composition also can be administered in the form of sustained-release formulations (see, e.g., U.S. Pat. No. 5,378,475) comprising, for example, gel foam, hyaluronic acid, gelatin, chondroitin sulfate, a polyphosphoester, such as bis-2-hydroxyethyl-terephthalate (BHET), and/or a polylactic-glycolic acid.

The dose of the gene transfer vector in the composition administered to the mammal will depend on a number of factors, including the size (mass) of the mammal, the extent of any side-effects, the particular route of administration, and the like. In one embodiment, the method comprises administering a “therapeutically effective amount” of the composition comprising the inventive gene transfer vector described herein. A “therapeutically effective amount” refers to an amount effective, at dosages and for periods of time necessary, to achieve a desired therapeutic result. The therapeutically effective amount may vary according to factors such as the extent of the disease or disorder, age, sex, and weight of the individual, and the ability of the gene transfer vector to elicit a desired response in the individual. The dose of gene transfer vector in the composition required to achieve a particular therapeutic effect typically is administered in units of vector genome copies per cell (gc/cell) or vector genome copies/per kilogram of body weight (gc/kg). One of ordinary skill in the art can readily determine an appropriate gene transfer vector dose range to treat a patient having a particular disease or disorder, based on these and other factors that are well known in the art. In one embodiment, the therapeutically effective amount may be between 1×10¹⁰ genome copies to 1×10¹³ genome copies for viruses.

In one embodiment, the composition having the gene transfer vector is administered once to the mammal. It is believed that a single administration of the composition may result in persistent expression in the mammal with minimal side effects. However, in certain cases, it may be appropriate to administer the composition multiple times during a therapeutic period to ensure sufficient exposure of cells to the composition. For example, the composition may be administered to the mammal two or more times (e.g., 2, 3, 4, 5, 6, 7, 8, 9, or 10 or more times) during a therapeutic period.

The present disclosure provides pharmaceutically acceptable compositions which comprise a therapeutically-effective amount of gene transfer vector comprising a nucleic acid sequence as described above.

### Routes of Administration, Dosages and Dosage Forms

Administration of the gene delivery vector or nanoparticles may be continuous or intermittent, depending, for example, upon the recipient's physiological condition, and other factors known to skilled practitioners. The administration of the gene delivery vector(s) or nanoparticles may be essentially continuous over a preselected period of time or may be in a series of spaced doses. Both local administration, e.g., intra joint, intranasal or intrathecal, and systemic administration are contemplated. Any route of administration may be employed, e.g., intravenous, intranasal or intrabronchial, or local administration, e.g., to a joint. In one embodiment, compositions may be delivered to a joint.

One or more suitable unit dosage forms comprising the gene delivery vector(s), which may optionally be formulated for sustained release, or nanoparticles, can be administered by a variety of routes including local, e.g., to a joint or intrathecal, oral, or parenteral, including by rectal, buccal, vaginal and sublingual, transdermal, subcutaneous, intravenous, intramuscular, intraperitoneal, intrathoracic, or intrapulmonary routes. The formulations may, where appropriate, be conveniently presented in discrete unit dosage forms and may be prepared by any of the methods well known to pharmacy. Such methods may include the step of bringing into association the vector with liquid carriers, solid matrices, semi-solid carriers, finely divided solid carriers or combinations thereof, and then, if necessary, introducing or shaping the product into the desired delivery system.

The amount of gene delivery vector(s) administered to achieve a particular outcome will vary depending on various factors including, but not limited to, the genes and promoters chosen, the condition, patient specific parameters. e.g., height, weight and age, and whether prevention or treatment, is to be achieved.

Vectors or nanoparticles may conveniently be provided in the form of formulations suitable for administration. A suitable administration format may best be determined by a medical practitioner for each patient individually, according to standard procedures. Suitable pharmaceutically acceptable carriers and their formulation are described in standard formulations treatises. e.g., Remington's Pharmaceuticals Sciences. By “pharmaceutically acceptable” it is meant a carrier, diluent, excipient, and/or salt that is compatible with the other ingredients of the formulation, and not deleterious to the recipient thereof.

Vectors of the present invention may be formulated in solution at neutral pH, for example, about pH 6.5 to about pH 8.5, or from about pH 7 to 8, with an excipient to bring the solution to about isotonicity, for example, 4.5% mannitol or 0.9% sodium chloride, pH buffered with art-known buffer solutions, such as sodium phosphate, that are generally regarded as safe, together with an accepted preservative such as metacresol 0.1% to 0.75%, or from 0.15% to 0.4% metacresol. Obtaining a desired isotonicity can be accomplished using sodium chloride or other pharmaceutically acceptable agents such as dextrose, boric acid, sodium tartrate, propylene glycol, polyols (such as mannitol and sorbitol), or other inorganic or organic solutes. Sodium chloride is useful for buffers containing sodium ions. If desired, solutions of the above compositions can also be prepared to enhance shelf life and stability. Therapeutically useful compositions can be prepared by mixing the ingredients following generally accepted procedures. For example, the selected components can be mixed to produce a concentrated mixture which may then be adjusted to the final concentration and viscosity by the addition of water and/or a buffer to control pH or an additional solute to control tonicity.

The vectors can be provided in a dosage form containing an amount of a vector effective in one or multiple doses. For viral vectors, the effective dose may be in the range of at least about 10⁷ viral particles, e.g., about 10⁹ viral particles, or about 10¹¹ viral particles. The number of viral particles added may be up to 10¹⁴. For example, when a viral expression vector is employed, about 10⁸ to about 10⁸⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results. The amount administered will vary depending on various factors including, but not limited to, the nucleic acid or vector chosen for administration, the disease, the weight, the physical condition, the health, and/or the age of the mammal. Such factors can be readily determined by the clinician employing animal models or other test systems that are available in the art. As noted, the exact dose to be administered is determined by the attending clinician but may be in 1 mL phosphate buffered saline. For delivery of plasmid DNA alone, or plasmid DNA in a complex with other macromolecules, the amount of DNA to be administered will be an amount which results in a beneficial effect to the recipient. For example, from 0.0001 to 1 mg or more, e.g., up to 1 g, in individual or divided doses, e.g., from 0.001 to 0.5 mg, or 0.01 to 0.1 mg, of DNA can be administered.

For example, when a viral expression vector is employed, about 10⁸ to about 10⁶⁰ gc of viral vector can be administered as nucleic acid or as a packaged virion. In some embodiments, about 10⁹ to about 10¹⁵ copies of viral vector, e.g., per 0.5 to 10 mL, can be administered as nucleic acid or as a packaged virion. Alternatively, the nucleic acids or vectors, can be administered in dosages of at least about 0.0001 mg/kg to about 1 mg/kg, of at least about 0.001 mg/kg to about 0.5 mg/kg, at least about 0.01 mg/kg to about 0.25 mg/kg or at least about 0.01 mg/kg to about 0.25 mg/kg of body weight, although other dosages may provide beneficial results.

By way of illustration, liposomes and other lipid-containing gene delivery complexes can be used to deliver one or more transgenes. The principles of the preparation and use of such complexes for gene delivery have been described in the art (see, e.g., Ledley, (1995); Miller et al., (1995); Chonn et al., (1995); Schofield et al., (1995); Brigham et al., (1993)).

Pharmaceutical formulations containing the gene delivery vectors or nanoparticles can be prepared by procedures known in the art using well known and readily available ingredients. For example, the agent can be formulated with common excipients, diluents, or carriers, and formed into tablets, capsules, suspensions, powders, and the like. The vectors of the invention can also be formulated as elixirs or solutions appropriate for parenteral administration, for instance, by intramuscular, subcutaneous or intravenous routes.

The pharmaceutical formulations of the vectors or nanoparticles can also take the form of an aqueous or anhydrous solution, e.g., a lyophilized formulation, or dispersion, or alternatively the form of an emulsion or suspension.

In one embodiment, the vectors or nanoparticles may be formulated for administration, e.g., by injection, for example, bolus injection or continuous infusion via a catheter, and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable vehicles and adjuvants which are well known in the art. It is possible, for example, to prepare solutions using one or more organic solvent(s) that is/are acceptable from the physiological standpoint.

For administration to the upper (nasal) or lower respiratory tract by inhalation, the vector is conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatine or blister packs from which the powder may be administered with the aid of an inhalator, insufflator or a metered-dose inhaler.

For intra-nasal administration, the vector may be administered via nose drops, a liquid spray, such as via a plastic bottle atomizer or metered-dose inhaler. Typical of atomizers are the Mistometer (Wintrop) and the Medihaler (Riker).

The local delivery of the vectors or nanoparticles can also be by a variety of techniques which administer the vector at or near the site of disease, e.g., using a catheter or needle Examples of site-specific or targeted local delivery techniques are not intended to be limiting but to be illustrative of the techniques available. Examples include local delivery catheters, such as an infusion or indwelling catheter, e.g., a needle infusion catheter, shunts and stents or other implantable devices, site specific carriers, direct injection, or direct applications.

The formulations and compositions described herein may also contain other ingredients such as antimicrobial agents or preservatives.

### Subjects

The subject may be any animal, including a human and non-human animals. Non-human animals includes all vertebrates, e.g., mammals and non-mammals, such as non-human primates, sheep, dogs, cats, cows, horses, chickens, amphibians, and reptiles, although mammals are preferred, such as non-human primates, sheep, dogs, cats, cows and horses. The subject may also be livestock such as, cattle, swine, sheep, poultry, and horses, or pets, such as dogs and cats.

The subject is generally diagnosed with the condition of the subject invention by skilled artisans, such as a medical practitioner.

The methods described herein can be employed for subjects of any species, gender, age, ethnic population, or genotype. Accordingly, the term subject includes males and females, and it includes elderly, elderly-to-adult transition age subjects adults, adult-to-pre-adult transition age subjects, and pre-adults, including adolescents, children, and infants.

Examples of human ethnic populations include Caucasians, Asians, Hispanics, Africans, African Americans, Native Americans, Semites, and Pacific Islanders. The methods of the invention may be more appropriate for some ethnic populations such as Caucasians, especially northern European populations, as well as Asian populations.

The term subject also includes subjects of any genotype or phenotype as long as they are in need of the invention, as described above. In addition, the subject can have the genotype or phenotype for any hair color, eye color, skin color or any combination thereof.

The term subject includes a subject of any body height, body weight, or any organ or body part size or shape.

### EXEMPLARY EMBODIMENTS

In one embodiment, a cationic nanoparticle formed of a mixture of a biodegradable or biocompatible synthetic polymer and a dendrimer, which nanoparticles have a diameter of about 125 nm to 1000 nm, is provided. In one embodiment, the cationic nanoparticle is formulated so that the dendrimer does not form a coat (layer) over a nanoparticle comprising the synthetic polymer. In one embodiment, the synthetic polymer comprises a polyolefin, silicone, polyacrylate, polyester, polyether, polyamide or polyurethane. In one embodiment, the synthetic polymer comprises a polyvinyl alcohol, polyglycolic acid, polyhydroxyalkanoate, polylactic acid, or co-polymers of polyglycolic acid and poly-lactic acid. In one embodiment, the dendrimer comprises a polyamidoamine, polyethyleneimine, polypropyleneimine, poly-L-lysine, or diethylaminoethyl dextran. In one embodiment, the dendrimer comprises polyvinyl alcohol, polyethylene glycol or carbosilane. In one embodiment, the nanoparticle does not include isolated nucleic acid, e.g., on the surface of the nanoparticle or complexed with the nanoparticle. In one embodiment, the nanoparticle does not include plasmid DNA. In one embodiment, the diameter of the nanoparticle is about 150 nm to about 600 nm. In one embodiment, the diameter of the nanoparticle is about 150 nm to about 250 nm. In one embodiment, the diameter of the nanoparticle is about 250 nm to about 500 nm. In one embodiment, the synthetic polymer is PLGA. In one embodiment, the dendrimer is PAMAM, e.g., G2, G3, G4, G5, G6 or G7.

In one embodiment, a lyophilized product comprising a plurality of the nanoparticles is provided, and optionally includes a cryoprotectant or carrier.

In one embodiment, a pharmaceutical composition comprising an amount of the nanoparticle effective as an adjuvant is provided, which optionally includes a pharmaceutically acceptable carrier.

In one embodiment, a method to enhance the immune response in a mammal is provided. In one embodiment, a method to inhibit or treat cancer in a mammal is provided. In one embodiment, the method includes comprising administering to the mammal an amount of an immunogen or a vector encoding the immunogen and an amount of the nanoparticle effective as an adjuvant. In one embodiment, the mammal is a human. In one embodiment, the vector is a viral vector. In one embodiment, the viral vector is an adenovirus, lentivirus, retrovirus, herpesvirus or adeno-associated viral vector. In one embodiment, the mammal has cancer. In one embodiment, the cancer is melanoma. In one embodiment, the tumor is a solid tumor. In one embodiment, the immunogen is a tumor-specific antigen. In one embodiment, the immunogen is a tumor-associated antigen. In one embodiment,
the immunogen is a microbial antigen. In one embodiment, the microbe is a virus, bacterium, yeast or fungus. In one embodiment, the immunogen or vector is administered at a site that is different than the administration site for the nanoparticles. In one embodiment, the immunogen is systemically administered. In one embodiment, the nanoparticles are locally administered. In one embodiment, the nanoparticles are systemically administered. In one embodiment, the immunogen is locally administered. In one embodiment, the method includes comprising administering one or more chemotherapeutic agents. In one embodiment, the method includes administering one or more checkpoint inhibitors.

The invention will be further described by the following non-limiting examples.

### Example 1

### Methods

### Adenovirus Synthesis

The attenuated serotype 5 adenoviruses (Ad5) used in these studies were manufactured by the Viral Vector Core (University of Iowa, Carver College of Medicine, Iowa City, IA) using a method previously described (Anderson et al., 2000). The viral DNA construct was engineered to express human tyrosinase-like protein-2 (TRP2) upon transduction of living cells. All Ad5 had a portion of the adenoviral genome deleted that included the left-hand terminal repeat, the packaging signal and E1A and E1B sequences, rendering the virus replication deficient.

### PLGA/PAMAM NP (PMG5, PMG4) Formulation

PLGA PAMAM nanoparticles (PM) were fabricated using a modified nanoprecipitation method. Briefly, 50 mg of 50:50 poly(D,L-Lactide-co-Glycolide) (PLGA, Resomer® RG 502) was dissolved in 5 mL acetone, then, 125 μL of polyamidoamine (PAMAM) dendrimer (ethylenediamine core, generation 5 or generation 4) was added to make a resulting PAMAM solution at a concentration of 0.125% w/v. PLGA/PAMAM solution and was allowed to drain into a 0.1% w/v PVA aqueous solution (Sigma Aldrich, MW 9000-10000 g/mol) under stirring from a syringe needle (G26) under its own weight. Following this, instantaneously formed PMs were stirred in a fume hood for 30 minutes. PMs were then transferred to a rotary evaporator (Buchi, Rotavapor R-300), which was set to 50 mBar for 50 rpm for 4 hours to evaporate organic solvents. Then PMs were washed with sterilized DI water in Amicon™ Ultra, centrifuge tubes (100,000 MWCO) 4 times by centrifuging 500×g for 1 hour. PMs were then frozen in a 10% sucrose solution at −80° C. for 24 hours, then lyophilized for 48 hours using a lyophilizer (Labconco, freezone—4.5 L). Finally, dry PMs were collected and stored in sealed containers until use. To evaluate the impact of involving the lyophilization process and adding the sucrose to the formulation, two other different formulations of PMs were prepared; one was freshly prepared (i.e., no freezing or lyophilization) while the other batch was prepared and lyophilized in the absence of cryoprotectant (e.g., containing no sucrose). PMs synthesized using PAMAM dendrimer (ethylenediamine core, generation 5) were designated as PMG5; and those synthesized using PAMAM dendrimer (ethylenediamine core, generation 4) were designated PMG4.

### PLGA/PAMAM G3 NP (PMG3) Formulation

Due to its high methanol content (20 wt. %), 125 μL PAMAM dendrimer (ethylenediamine core, generation 3) was placed in a rotary evaporator (Buchi, Rotavapor R-300), which was set to 50 mBar for 50 rpm for 30 minutes to evaporate organic solvents, PAMAM was then resuspended in 125 μL of DMSO. This solution was then used to synthesize PLGA PAMAM nanoparticles (PM) using the fabrication method listed above with generation 4 and 5 PAMAM.

### Characterization of PM Formulation

PMG5 were imaged using transmission electron microscopy (TEM) (JEOL, JEM-1230). Briefly, 0.1 mg/mL of lyophilized PMG5 was added to 1.5% sterile filtered phosphotungstic acid at a ratio of 1:1.

The resulting solution was placed on carbon coated TEM grids and imaged to analyze morphology of PMG5. PMG5 were further characterized by assessing the average hydrodynamic diameter, polydispersity index (PDI), and net surface charge using a Zetasizer Nano ZS (Malvern) as previously described (Wafa et al., 2017; Wafa et al. 2019a; Wafa et al., 2019b; Wafa et al., 2019c). In addition, the shape and surface morphology of PMG5 were further examined using a Hitachi scanning electron microscope (SEM) (Hitachi High-Technologies) (Wafa et al., 2017; Wafa et al. 2019a; Wafa et al., 2019b; Wafa et al., 2019c). PLGA, PAMAM and PMG5 were also characterized for heat flow properties by analyzing the thermograms obtained from the differential scanning calorimeter (DSC Q20) equipped with a refrigerated cooling system (RCS90) (TA Instruments), as previously described (Wafa et al., 2019c). All samples were sealed in standard aluminum sample pans covered with lids. An empty sealed aluminum pan covered with a lid was used as a reference. Pure dry nitrogen (set at 20 psi pressure and 40 mL/min flow rate) was used as a purge gas. Samples were heated from 0° C. to 100° C. at 5° C./min heating rate.

### Degradation Studies

In this experiment, 5 mg of lyophilized PMG3, PMG4 and PMG5 were dissolved in 5 mL of nanopure water. The resulting solutions were placed at room temperature and at 37° C. and agitated at 300 rpm for 42 days. Samples were taken at various time points (every day for 3 days then every week for two weeks then every two weeks) and the size, zetapotential and PDI were recorded using the Zetasizer Nano ZS (Malvern).

### COG-PM (G5) Complexation

To evaluate the amount of CpG B 1826 (Integrated DNA Technologies, Inc.) that was required to complex completely with PMs, a constant amount of lyophilized particles (1 mg) was weighed out and dispersed into 7 samples (1 mg per sample) in ultrapure water (Gibco, Thermo Fischer Scientific). To each resulting solution incremental amounts of CpG B were added (i.e., solution #1 contained 0.5 mg CpG B, solution #2 contained 0.25 mg CpG B, etc.). Each solution was then vortexed for 2 minutes, then allowed to equilibrate at room temperature for 2-3 minutes. The ZP of the resulting solution was measured using the Zetasizer Nano ZS (Malvern). In addition, an aliquot from each solution was added to a 10% agarose gel, and gel electrophoresis was performed.

### Evaluation of the Effect of Lyophilization

Due to the intrinsic fluorescence of PAMAM, cellular uptake behavior of PMG5 could be directly analyzed by fluorescence microscopy and flow cytometry, without additional fluorescence labeling (Tsai et al., 2011). Herein, quantitative and qualitative cellular uptake of PMG5 by BMDCs was studied. For tracking the in vitro cellular uptake of PMG5, BMDCs were incubated with 0.12 mg of different formulations of PMG5 as follows: (i) non-lyophilized PM (freshly prepared), (ii) lyophilized PMs in the presence of sucrose, (iii) lyophilized PMs without sucrose, and untreated BMDCs as a control (iv). Cells were then collected (without using trypsin; instead vigorous flushing was implemented) and centrifuged (230×g) for 5 minutes at 4° C. All cell samples were run through a BD FACScan flow cytometer (Becton, Dickinson, Franklin Lakes, NJ) in triplicate and data were analyzed with FlowJo software (Tree Star, Ashland, OR).

### Study the Dose-Uptake Relationship

In this study, BMDCs were incubated with four different doses (0.06, 0.12, 0.18, and 0.24 mg) of lyophilized PMG5 with sucrose to assess the effect of the dose on the uptake capacity. After incubation for 48 hours, cells were collected, and samples were acquired using a FACScan flow cytometer (Becton-Dickinson).

In this experiment, the uptake capacity of PMs was assessed on BMDCs using both serum-free media and media-containing serum. BMDCs were initially seeded in 12-well plate in media-containing serum, as described above. Prior to adding the PMG5, the media was removed, and fresh serum-free media was added. This was followed by adding the PMG5 (lyophilized with sucrose) to the BMDCs. Cells were then collected and analyzed by FACScan.

### Mechanistic Study of the Uptake of PMs

For efficient optimization of the PMG5, it is crucial to profile the cellular uptake pathway since this largely determines its intracellular processing and subsequent activity. Therefore, this experiment was performed to study the cellular uptake mechanisms and determine if uptake of PMG5 by BMDCs could be inhibited by any of the inhibitors of endocytosis pathways. In general, endocytosis is an energy dependent process that can be delineated as clathrin-mediated endocytosis, caveolae-mediated endocytosis, pinocytosis, and phagocytosis. In this study, BMDCs were incubated with different endocytosis pathway inhibitors as shown in Table 1. This was followed by adding the PMG5 (0.12 mg) and incubation for 3 hours.

TABLE 1
List of endocytosis pathway inhibitors.

| | | Concentration | Treatment |
|---|---|---|---|
| Inhibitor | Function | Used | Time |
| Sucrose | Inhibits Clathrin- | 400 μM | 1 hour prior |
| | mediated endocytosis | | adding PMG5 |
| Methyl-β- | Inhibits Caveolae- | 1 mM | 1 hour prior |
| Cyclodextrin | dependent endocytosis | | adding PMG5 |
| Amiloride | Inhibits | 2 mM | 10 minutes prior |
| | macropinocytosis | | to adding PMG5 |

Harvesting Bone Marrow Derived Dendritic Cells (BMDCs) and BMDC activation Studies

Femurs and tibia were harvested from C57BL/6J mice and then the bone marrow was flushed with Roswell Park Memorial Institute (RPMI) 1640 cell culture media supplemented with 0.01 M HEPES buffer (Gibco, Thermo Fischer Scientific), 1 mM sodium pyruvate (Gibco, Thermo Fischer Scientific), 1×glutamax (Gibco, Thermo Fischer Scientific), 50 mM 2-mercaptoethonal (Sigma Aldrich), 0.5 mg/mL gentamycin sulfate (IBI Scientific), and 10% fetal bovine serum (Atlanta biologicals). BMDCs were then counted and seeded at a density of 2×10⁶ cells in 10 mL complete RPMI medium with 20 ng/mL GM-CSF (granulocyte monocyte colony stimulating factor) (Peprotech) at 37° C. with 5% CO₂ in a bacterial Petri dish. At day 3 (72 hours after seeding) 10 mL of RPMI complete media with 20 ng/mL of GM-CSF was added. On days 6 and 8, 10 mL of cell culture supernatant was harvested and spun at 230×g, old media was aspirated and 10 mL of fresh media with 20 ng/mL GM-CSF was added. On day 10, cells were transferred to 12-well plates at a density of 1×10⁵ cell/well and allowed to equilibrate for at least 6 hours before incubating with experimental groups (Table 1) then for 2 days with in 2 mL complete media and experimental groups.

Supernatants from the cells were then collected and stored for later use in Luminex multiplex cytokine assays, and BMDCs were then washed from the plate with ice-cold 1×DPBS (Gibco, Thermo Fischer Scientific). BMDCs were collected, washed with FACS buffer (1×DPBS, 0.1% sodium azide and 5% FCS) and transferred in a 96 well U-bottom plate (Cell star, Greiner). Cells were then incubated with a 1/100 dilution of anti-mouse CD16/CD32 (clone 93, Invitrogen) for 15 minutes. BMDCs were then incubated with anti-mouse CD11c FITC (clone N418, Invitrogen), anti-mouse CD40 APC (clone 3/23, Biolegend), anti-mouse CD80 APC (16-10A1), anti-mouse MHC class I (H-2Kb) PE (AF6-88.5.5.3, Invitrogen) for 30 minutes. BMDCs were then washed twice with FACS buffer and resuspended in 100 μL of cytofix buffer (BD Biosciences) and allowed to incubate for 10 minutes on ice in the dark. Next, 100 μL of 1×PermVwash solution (BD Biosciences) was added to each well, then cells were spun at 660×g and resuspended in FACS buffer and stored until they were analyzed using the flow cytometer (FACSCalibur, Becton Dickson) and FlowJo software (TreeStar, OR).

### Cell Viability Assay (MTS Assay)

B16.F10 or HEK 293 cells were removed from liquid nitrogen storage and grown to confluency in Dulbecco's Modified Eagle Medium (DMEM) (Thermo Fisher Scientific) supplemented with 0.01 M HEPES buffer (Gibco, Thermo Fischer Scientific), 1 mM sodium pyruvate (Gibco, Thermo Fischer Scientific), 1× glutamax (Gibco, Thermo Fischer Scientific), 0.5 mg/mL gentamycin sulfate (IBI Scientific, IA), and 10% fetal bovine serum (Atlanta biologicals) at 37° C. with 5% CO₂ in a T-75 flask (CellTreat, Pepperell, MA). Cell Medium was aspirated, and cells were washed with ×DPBS (Gibco, Thermo Fischer Scientific) and incubated with 2 mL 0.025% trypsin and 0.01% EDTA (Gibco, Thermo Fischer Scientific) for 2 minutes. Then 9 mL of complete cell culture medium was added, and cells were centrifuged at 230×g. Cell culture media was then aspirated, and cells resuspended in fresh media and plated in a 24 well plate (CellTreat, Pepperell, MA) at 1×10⁵ cells in each well with 1 mL of complete medium. Cells were then allowed to adhere for 4 to 8 hours, after which cells were incubated with designated amounts of PM for 48 hours. Cells were then washed with 1×DPBS and incubated with 500 μL or media MTS cocktail (1:4 MTS reagent to complete media) (Promega, MI) for 1 hour. Media MTS cocktail was then harvested, and absorbance read at 490 nm.

### Unilateral Tumor Challenge and Treatment Protocol

6-8-week-old, female C57/Bl6J mice were purchased from Jackson Laboratories and housed in the University of Iowa animal care facility for a minimum of 1 week before use. Mice were then randomly divided into the following groups at 9 mice per group.

TABLE 2
Table Listing Experimental Groups Used in Animal
Studies and In vitro BMDC Activation Studies.

| Group Name | Group Description | Dose (per mouse in 100 μL) |
|---|---|---|
| Naïve | Untreated Mice | — |
| Ad5-TRP2 | Adenovirus Serotype 5 | 1 × 10⁸ PFUs |
| | encoding tyrosinase | |
| | related protein-2 | |
| PMG5 | Empty PLGA/PAMAM | 1.6 mg |
| | G5 NPs (PMG5) | |
| CpG | Soluble CpG B | 50 μg |
| CpGPMG5 | CpG B complexed with | 50 μg of CpG with 1.6 mg |
| | PMG5 | PMG5 |
| Ad5/PMG5 | Ad5-TRP2 with empty | 1 × 10⁸ PFUs with 1.6 mg |
| | PMG5 | of PMG5 |
| Ad5/CpG | Ad5-TRP2 with soluble | 1 × 10⁸ PFUs with 50 μg |
| | CpG B | of CpG |
| Ad5/PMG3 | Ad5-TRP2 with empty | 1 × 10⁸ PFUs with 1.6 mg |
| | PMG3. | of PMG3 |
| Ad5/PMG4 | Ad5-TRP2 with empty | 1 × 10⁸ PFUs with 1.6 mg |
| | PMG4. | of PMG4 |
| Ad5/PMG3 | Ad5-TRP2 with empty | 1 × 10⁸ PFUs with 1.6 mg |
| | PMG3. | of PMG3. |
| Ad5/PMG3 | Ad5-TRP2 with empty | 1 × 10⁸ PFUs with 1.6 mg |
| CPB | PMG3 and αPD-1 | of PMG3 with 100 μg of |
| | and α4-1BB. | αPD-1 and 100 μg of α4-1BB. |
| Ad5/PMG4 | Ad5-TRP2 with empty | 1 × 10⁸ PFUs with 1.6 mg |
| CPB | PMG4 and αPD-1 | of PMG4 with 100 μg of |
| | and α4-1BB. | αPD-1 and 100 μg of α4-1BB. |
| Ad5/PMG5 | Ad5-TRP2 with empty | 1 × 10⁸ PFUs with 1.6 mg |
| CPB | PMG5 and αPD-1 | of PMG5 with 100 μg of |
| | and α4-1BB. | αPD-1 and 100 μg of α4-1BB. |

Mice were then challenged on the dorsal right flank with 2×10⁵ live B16.F10 cells in 100 μL serum-free DMEM complete media. The following day, mice designated to receive Ad5-TRP2 were given a single dose contralaterally at 1×10⁸ PFUs. On days 8, 11, and 13 post-tumor challenge (PTC), mice were given their designated treatment groups under anesthesia at the site of tumor inoculation on the right dorsal flank of the mouse. Tumor volumes and mice weights were recorded every 2-3 days. At 60 days post-initial tumor challenge, mice that did not develop tumors initially were re-challenged with 2×10⁵ cells on the dorsal right flank. On days 8, 11, 13, 16 and 18 PTC, select groups were administered with αPD1 (100 μg/mouse/administration) and/or α4-1BB (intraperitoneally (IP) at 100 μg/mouse/administration). Endpoint criteria were met when tumors reached 20 mm in length or width or 10 mm in height. Tumors were assumed to be ellipsoid in shape and volumes were recorded by measuring the length width and height with calipers and calculated using the formula:

$$Volume = ( length \times width \times height ) \times \frac{π}{6}$$

### Ex-Vivo Staining of TRP2-Specific CD8+T Lymphocytes

At 15 days PTC, 180 μL of blood was collected by submandibular bleeding and mixed with ACK buffer (150 mM NH4Cl, 10 mM KHCO3, 0.1 mM Na2EDTA) to lyse red blood cells. After a 10-minute incubation at room temperature, cells were washed (centrifuged at 230×g twice) using complete growth media. The cells were resuspended at <10⁷/mL (in ice cold PBS containing 2% v/v FCS and 0.05% sodium azide FACS buffer) in 96-well V-bottomed trays. Cells were then centrifuged at 4° C. at 230×g for 5 minutes, then the media was aspirated leaving the cell pellet in the plate. Cell pellets were then resuspended in 50 μL anti-mouse CD16/CD32 (1/100, clone 93, Invitrogen) and incubated on ice for 15 minutes. Then, 50 μL of diluted (1/100 in FACS buffer) tetramer stain was mixed in with FC block and then cells were incubated on ice in the dark for 30 minutes. Next, 100 μL of antibody cocktail [anti-CD8a-FITC (1/400, clone 53-6.7, ebiosciences), anti-CD3-PECy5 (1/200, clone 145-2C11, ebiosciences)] was added to the cells, and cells were then incubated for 20 minutes on ice in the dark. Cells were then washed twice with FACS buffer and resuspended in 100 μL cytofix solution and incubated on ice in the dark for 10 minutes. Finally, 100 μL of 1×Perm awash solution (BD Biosciences) was added to each well, then cells were centrifuged at 660×g, resuspended in FACS buffer and stored until analyzed using the flow cytometer (FACSCalibur, Becton Dickson) and FlowJo (TreeStar, OR) software within 2 weeks of fixing.

### Results

### Particle Synthesis, Characterization and Uptake

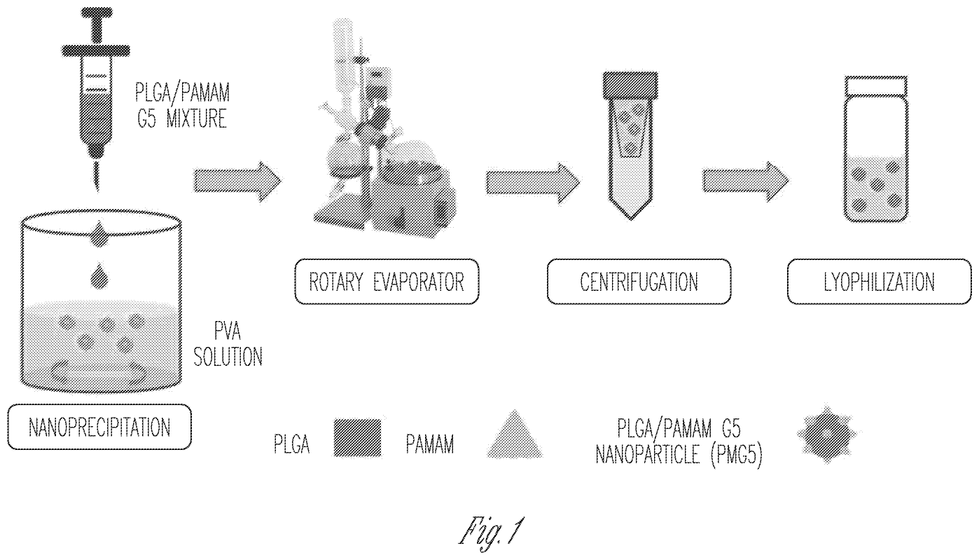

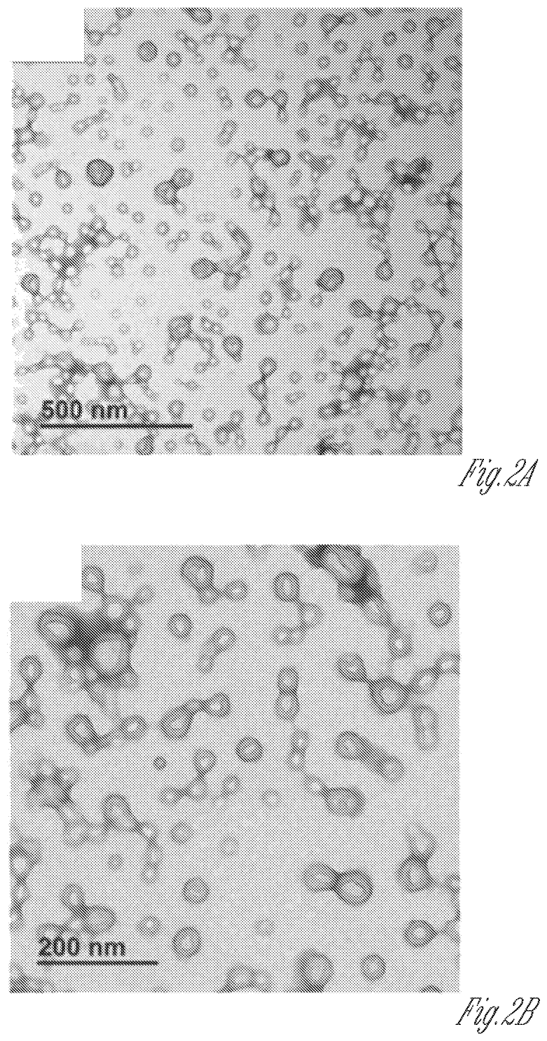

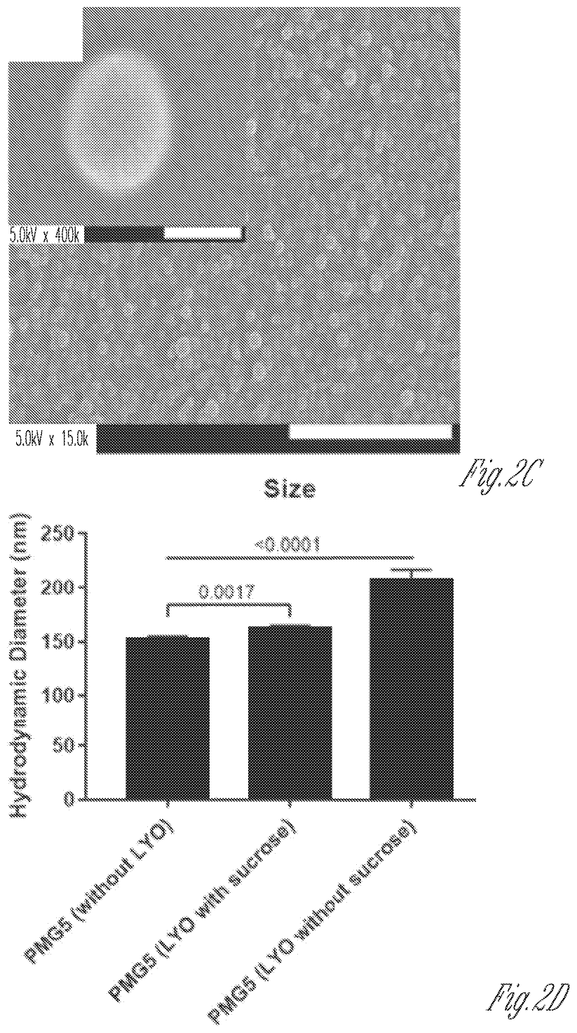

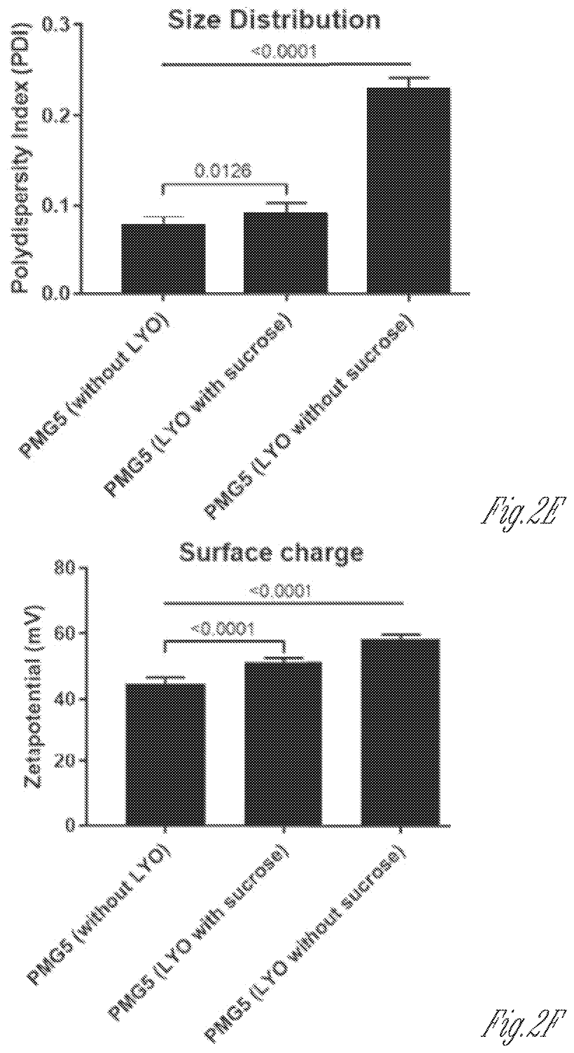

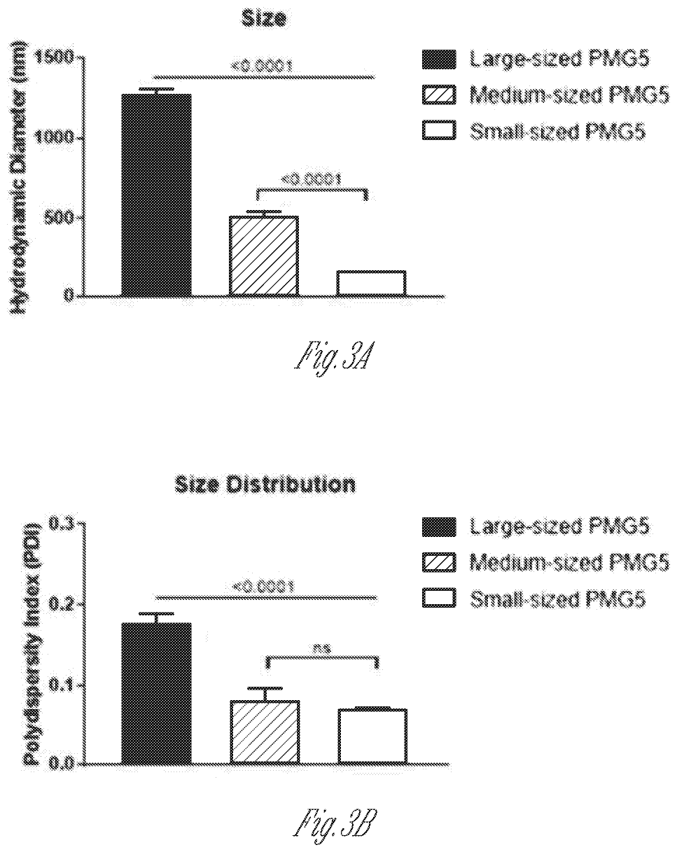

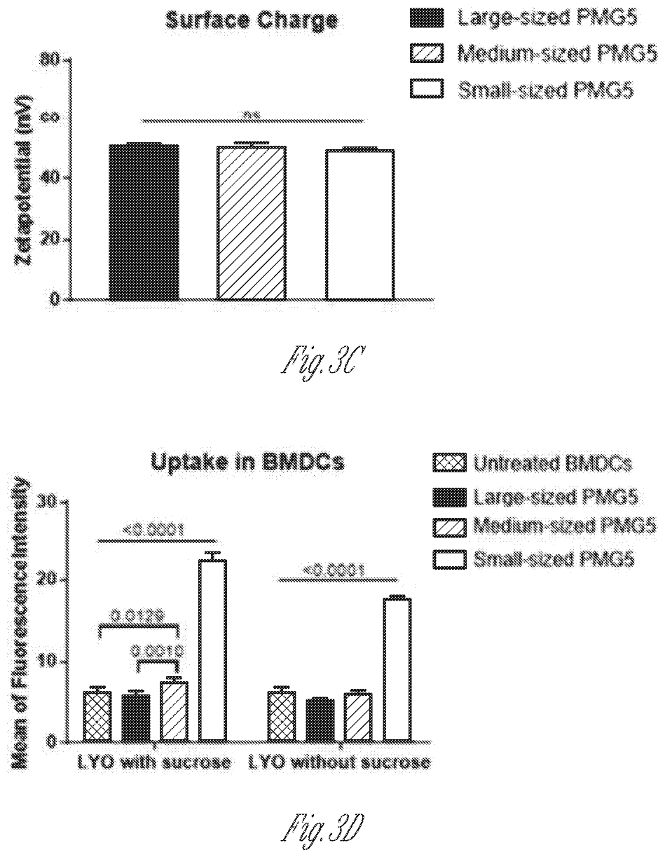

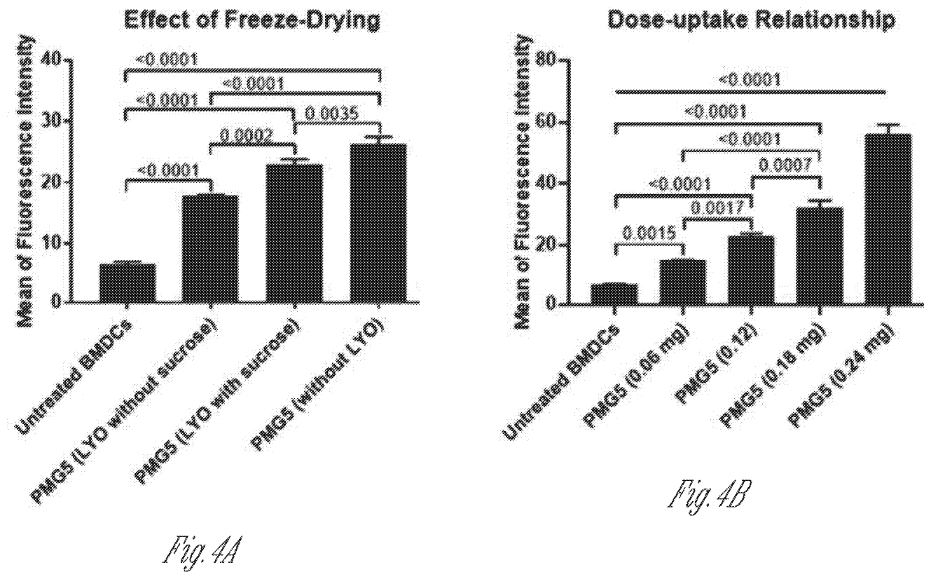

PAMAM can be obtained in different generations referring to the degree of branching from the core and directly relating to the amount of surface amines present on the PAMAM. Initially, PLGA-PAMAM nanoparticles (PMG5) were synthesized using the nanoprecipitation method summarized in (FIG. 1) from a PLGA and PAMAM (ethylenediamine core, Generation 5) mixture. TEM and SEM images showed that PMG5 were spherical with smooth surfaces (FIGS. 2A-C). Fabricating PMG5 prior to each use is likely not feasible for translation into the clinic. Thus, to evaluate the stability of PMG5 under appropriate storage conditions, PMG5 were lyophilized (freeze dried) with and without sucrose, a cryoprotectant. The lyophilization of PMG5 without sucrose resulted in significant aggregation as indicated by an increase in the size and PDI of the PMG5 (FIGS. 2D and 2E). This effect was circumvented by adding a cryoprotectant, sucrose, to the formulation which resulted in a decrease in size and polydispersity index (PDI) when compared to PMG5 lyophilized without sucrose. Also, it was observed that all PMG5 have a positively charged surface (FIG. 2F). It has been previously established that PAMAM dendrimers exhibit intrinsic fluorescence in the blue region with a slight shift in lambda max with increasing generations (Konopka et al., 2018; Wade et al., Caminati et al., 1990). Taking advantage of this feature, the intrinsic fluorescence of PAMAM was used to evaluate uptake of PMG5 by bone marrow-derived dendritic cells (BMDCs) and fluorescence (an indicator of uptake) was assessed by flow cytometry. Results shown in FIG. 3D demonstrate clearly that BMDCs incubated with small sized PMG5 (163.9±0.61 nm) had significantly greater fluorescence (due to the PMG5 being taken up by BMDCs) than BMDCs incubated with larger submicron (523.9 t 15 nm) and micro-sized particles (1278.3±27 nm) (also made from PLGA and PAMAM). Interestingly, differences between the fluorescence of untreated BMDCs and BMDCs treated with at least the medium particles may only be evident when BMDCs were treated with PMG5 particles lyophilized with sucrose.

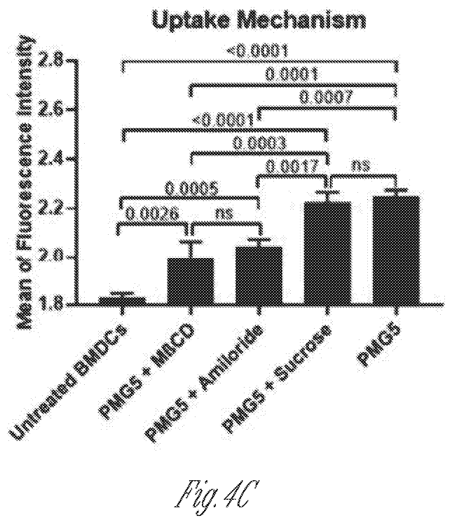

The effect of using sucrose as a stabilizer during lyophilization on small PMG5 uptake was further evaluated by incubating BMDCs with freshly prepared PMG5 and PMG5 lyophilized with and without sucrose. It was observed that small PMG5 lyophilized with sucrose resulted in more efficient uptake of PMG5 when compared to PMG5 lyophilized without sucrose (FIG. 4A). Also, as shown earlier (FIG. 2B), sucrose maintained the stability of size and size distribution. Consequently, small PMG5 that were lyophilized with sucrose were used in subsequent studies. Treatment of BMDCs with different doses of PMG5 revealed a dose-dependent rate of uptake (FIG. 4B). The uptake mechanism of PMG5 was studied by incubating the PMG5 nanoparticles with BMDCs pretreated with inhibitors of three distinct pathways of endocytosis; amiloride (inhibitor of macropinocytosis), methyl-O-cyclodextrin (MPCD) (inhibitor of clathrin-independent (CIE) endocytosis), and high concentration (400 μM) of sucrose (inhibitor of clathrin mediated endocytosis). As expected, incubation of BMDCs (not treated with any inhibitor) with PMG5 displayed a significant increase in the mean fluorescence intensity (MFI) compared to untreated BMDCs (cells not incubated with PMG5) (FIG. 4C). However, incubation of PMG5 with BMDCs pretreated with a high concentration of sucrose did not result in a significant reduction in the MFI when compared to BMDCs (untreated with any inhibitor) incubated with PMG5. Interestingly, PMG5 incubated with BMDCs pretreated with either amiloride or MPCD exhibited a significant reduction in the MFI in comparison to PM incubated with BMDCs. This indicated that both amiloride and MPCD inhibit the uptake of PMG5.

### PMG5 Thermal Stability

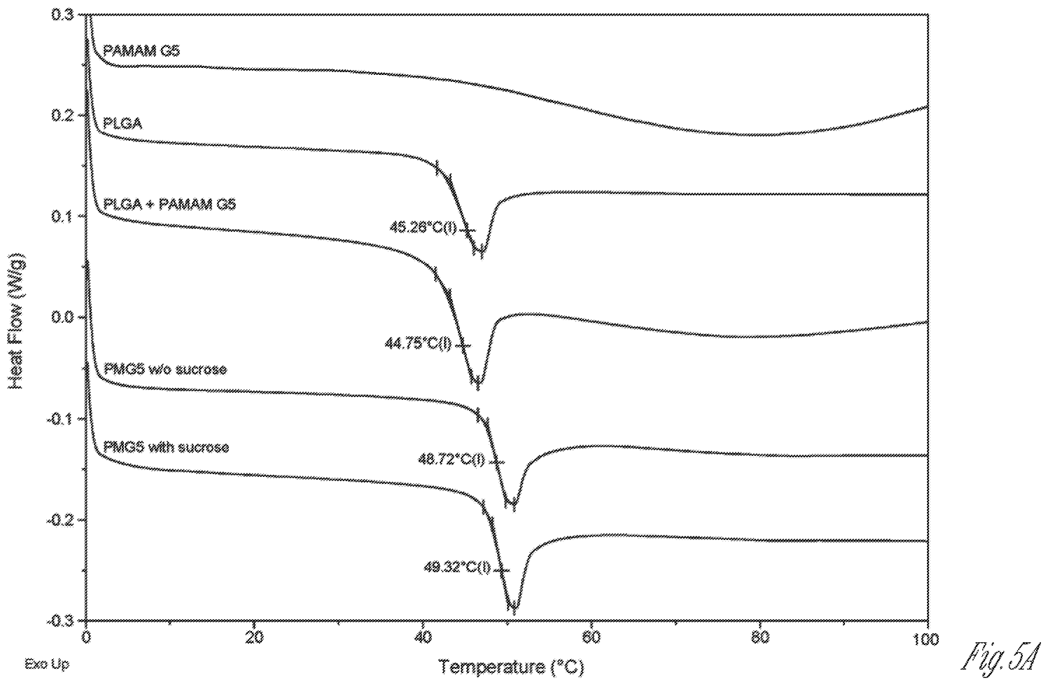

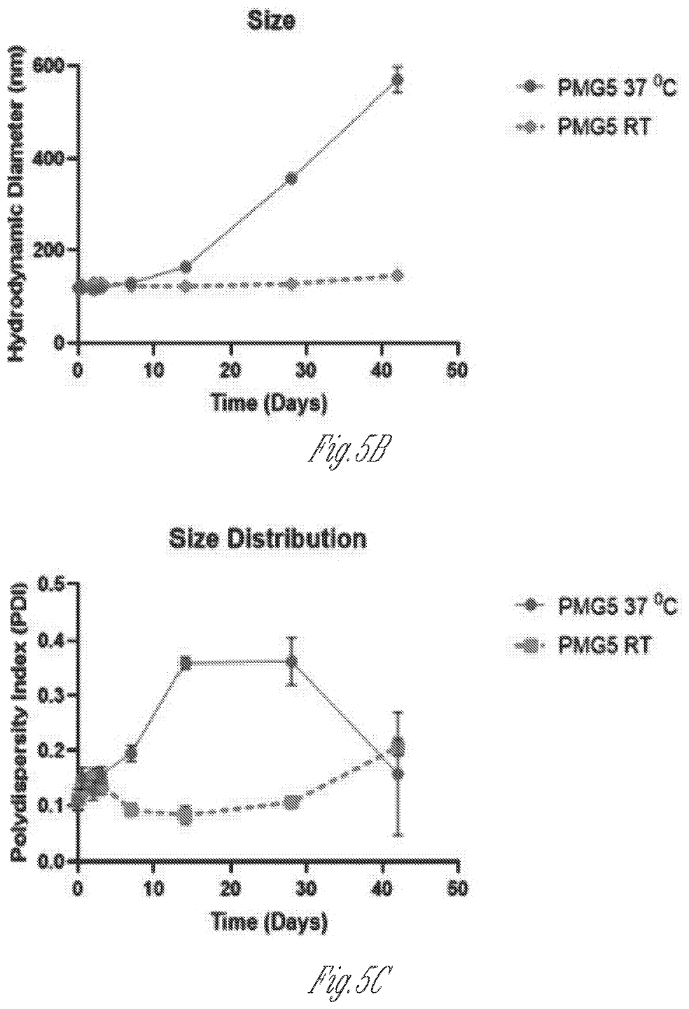

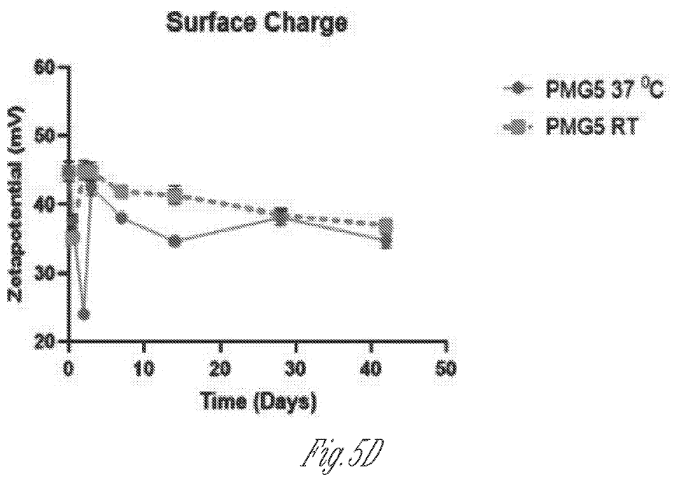

A DSC thermogram of the PLGA indicated that the polymer had a glass transition temperature of about 45° C. (representing the amorphous region of PLGA) (FIG. 5A). Adding the PAMAM G5 to PLGA (i.e., physical mixture) had no effect on the thermal properties of the PLGA. Surprisingly, PLGA formulated into nanoparticles (with and without sucrose) using the nanoprecipitation technique did not exhibit any thermal changes when compared to unprocessed PLGA; except for a slight shift in the glass transition temperature to approximately 49° C. The DSC thermogram demonstrated that the PMG5 formulation is thermally stable at the ambient temperature since it did not display any thermal events in the range 0-49° C. It is likely that the highly positive surface charge of PMG5 helps to prevent aggregation in solution at room temperature due to repulsive forces (FIGS. 5B and 5D). At 37° C., PMG5 dispersed in water had no significant change in size, PDI and surface charge for at least 10 days of stirring at 300 rpm (FIGS. 5B, SC and 5 D) after which hydrodynamic size did increase most likely to aggregation.

### PMG5 Adjuvant Loading and Cytotoxicity

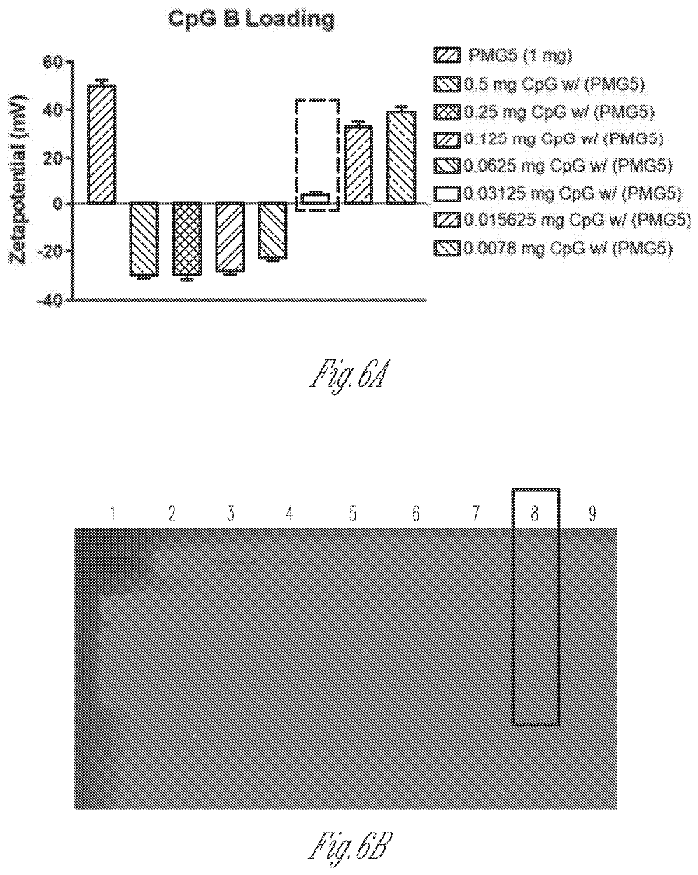

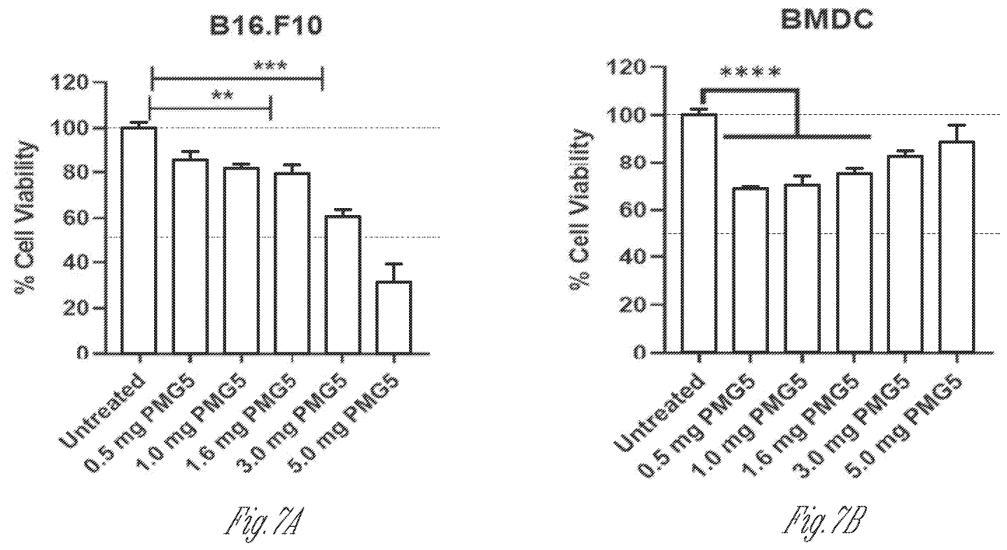

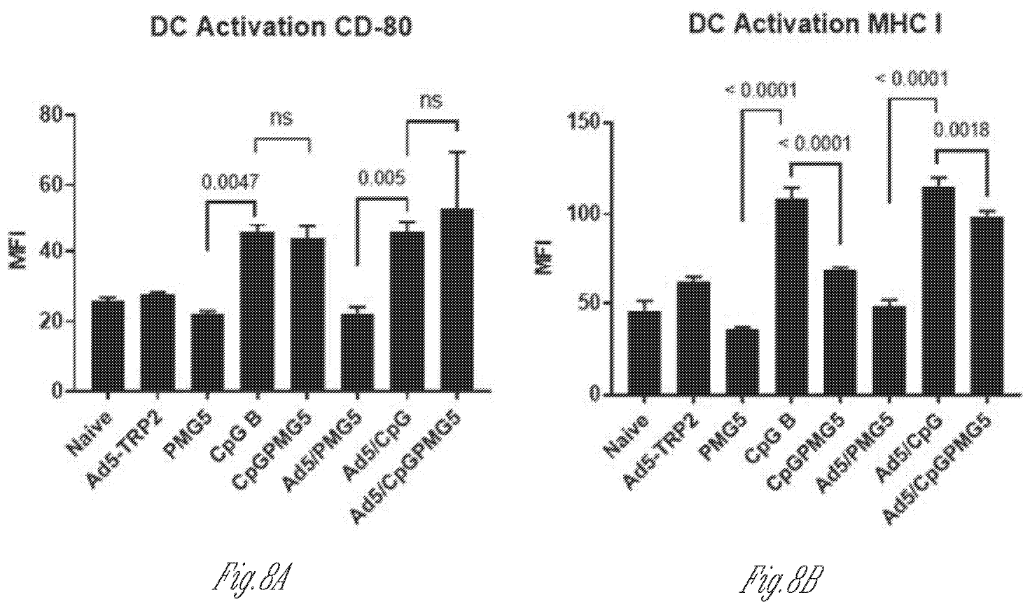

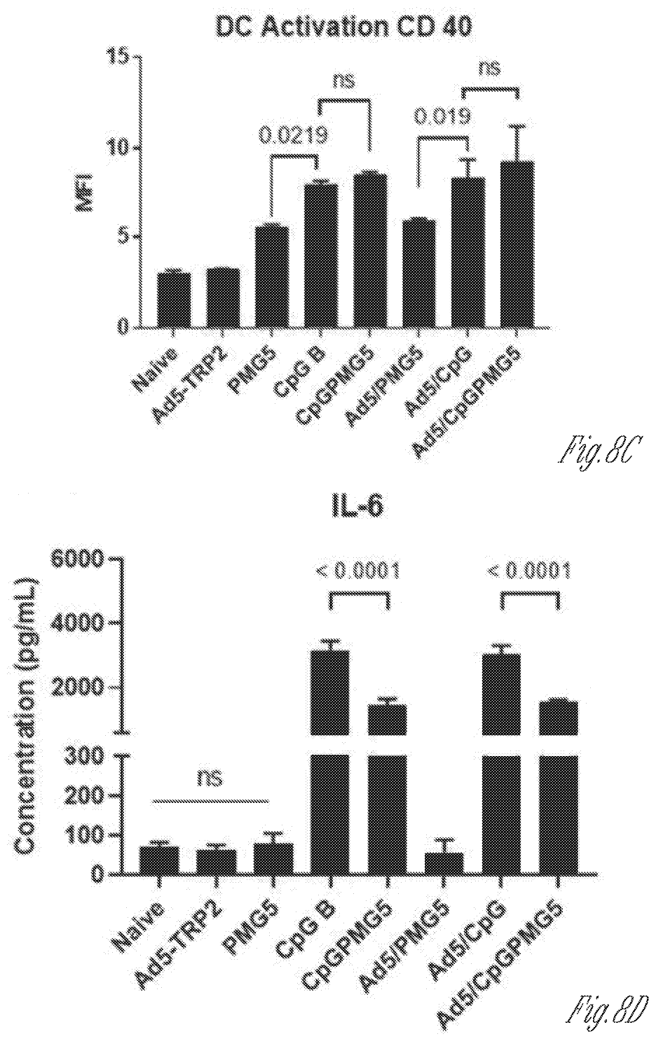

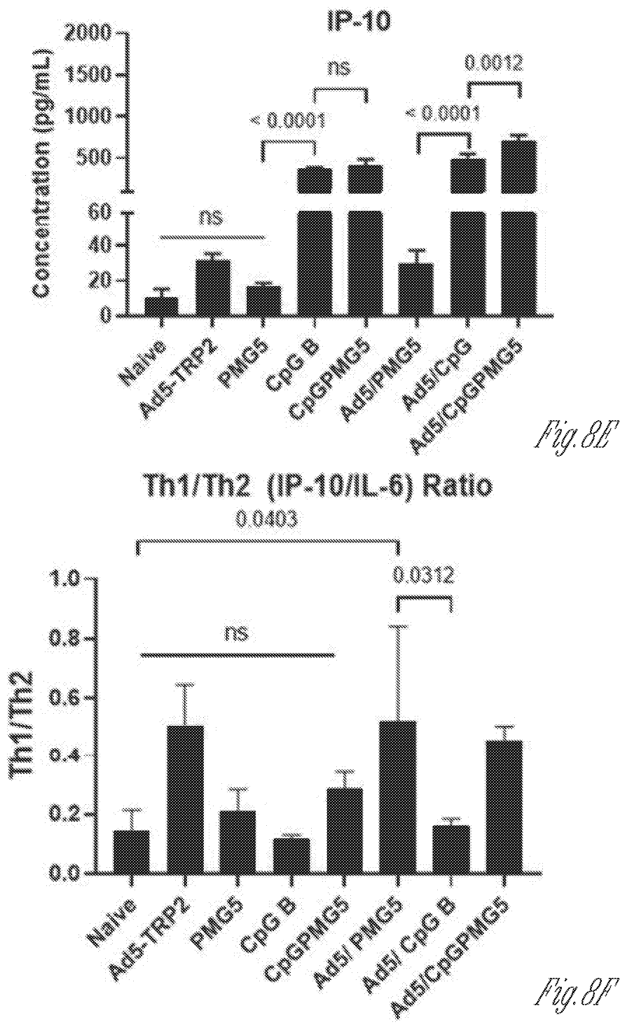

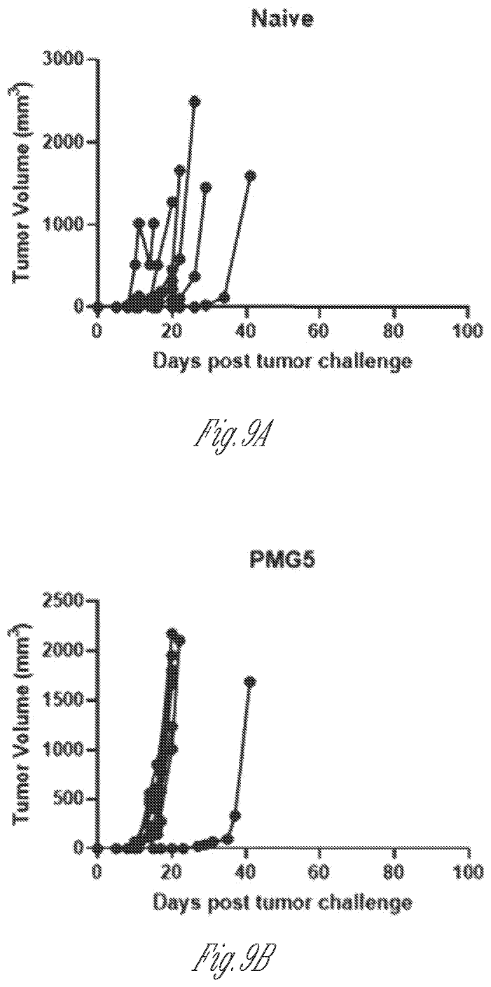

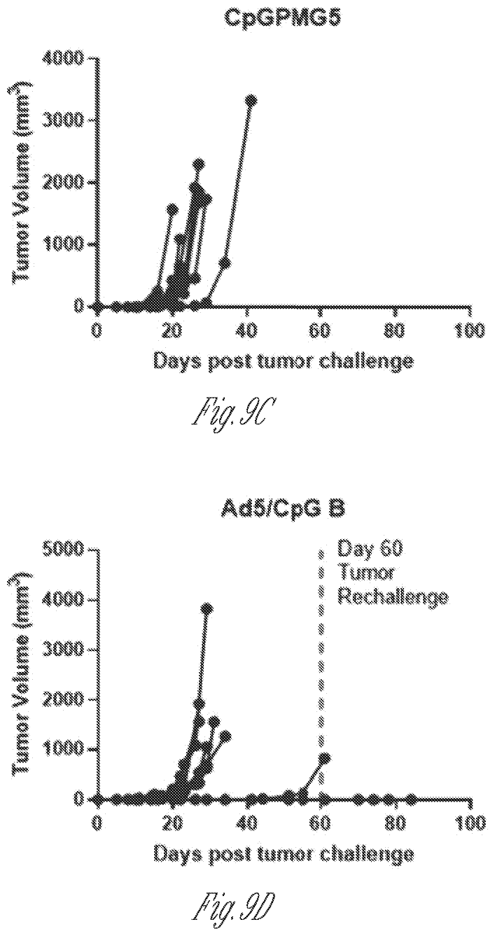

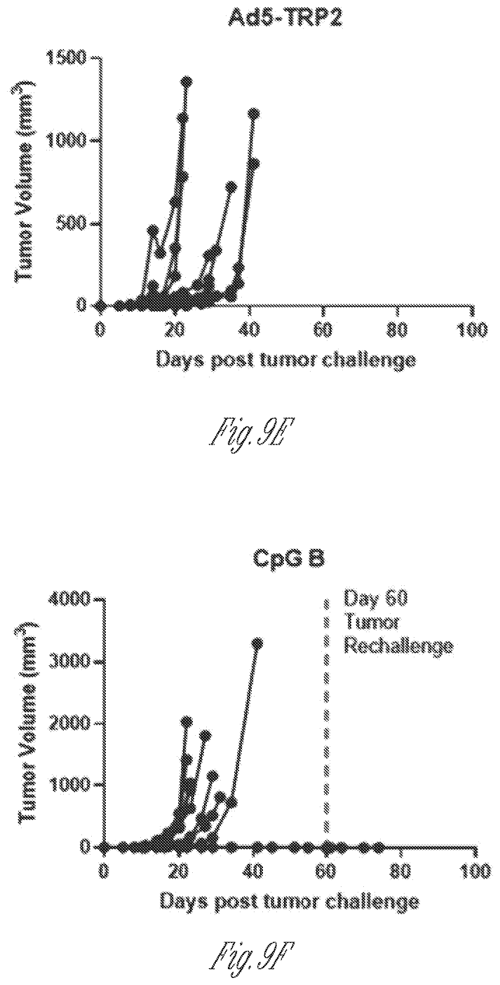

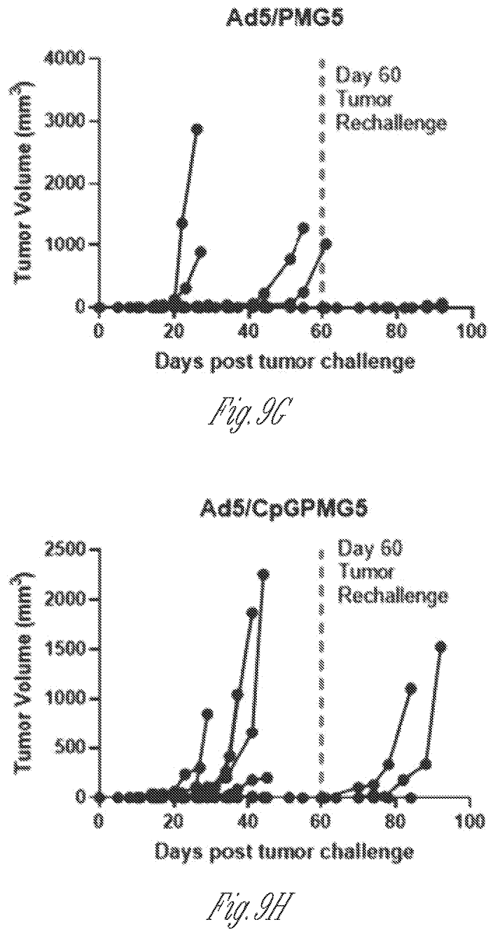

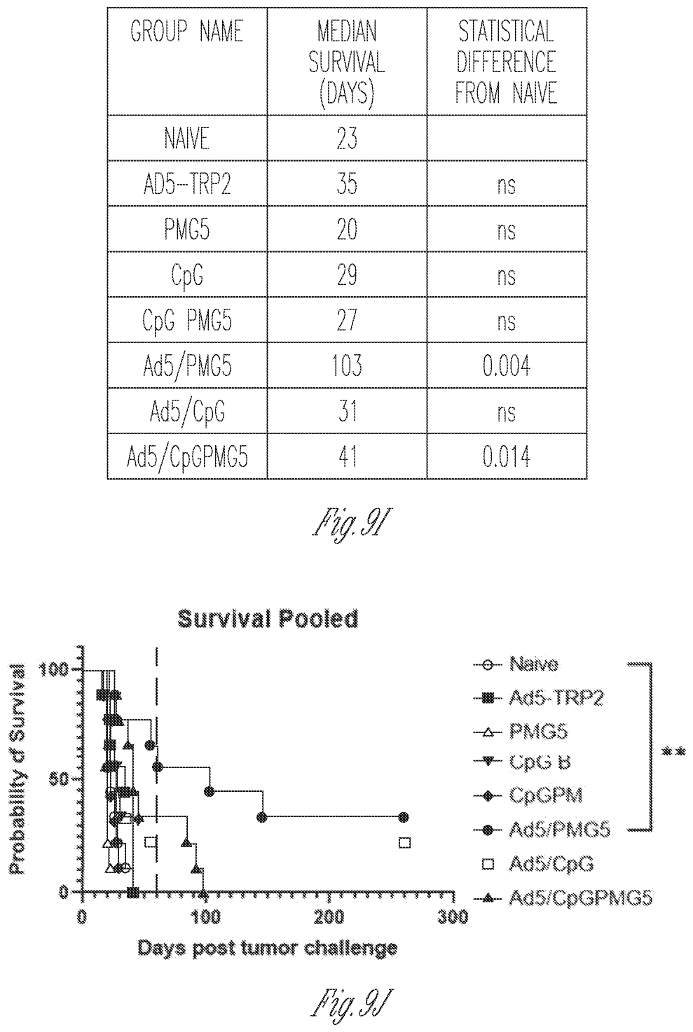

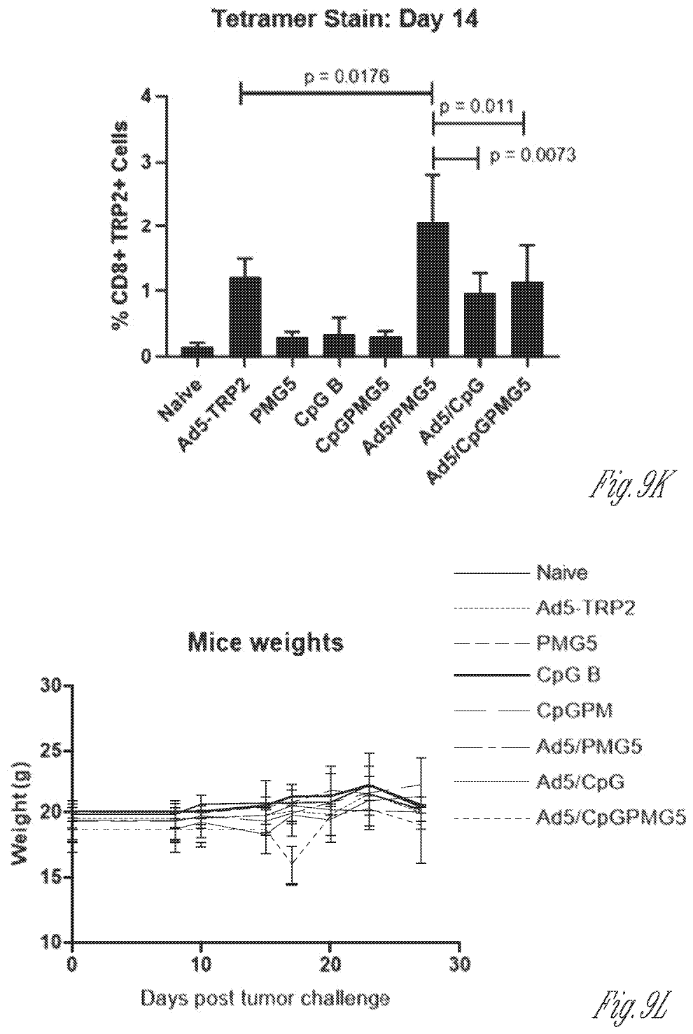

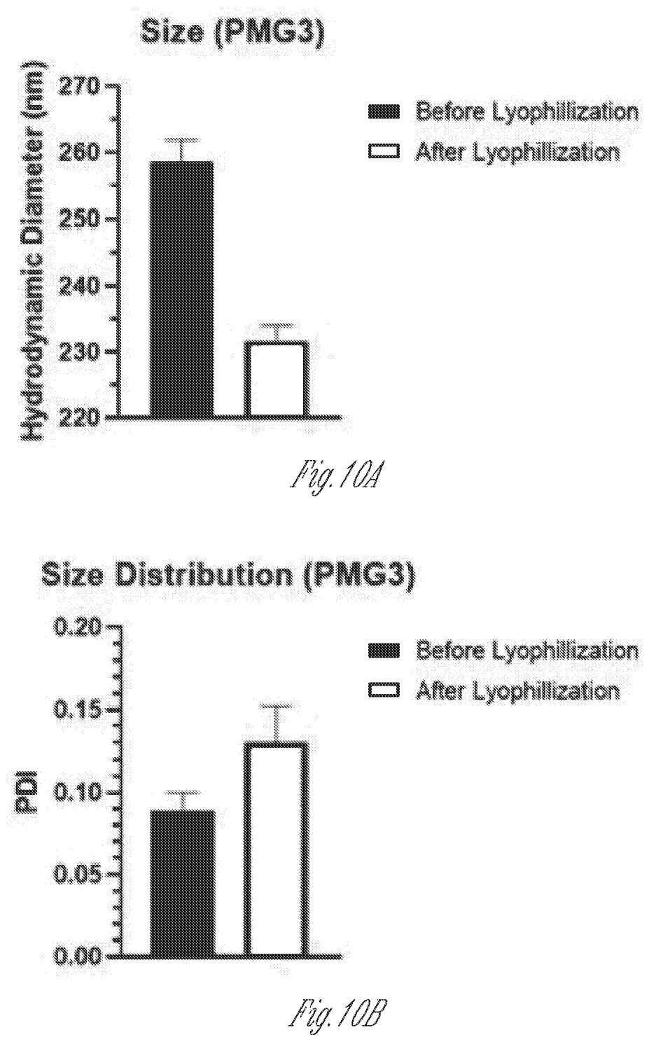

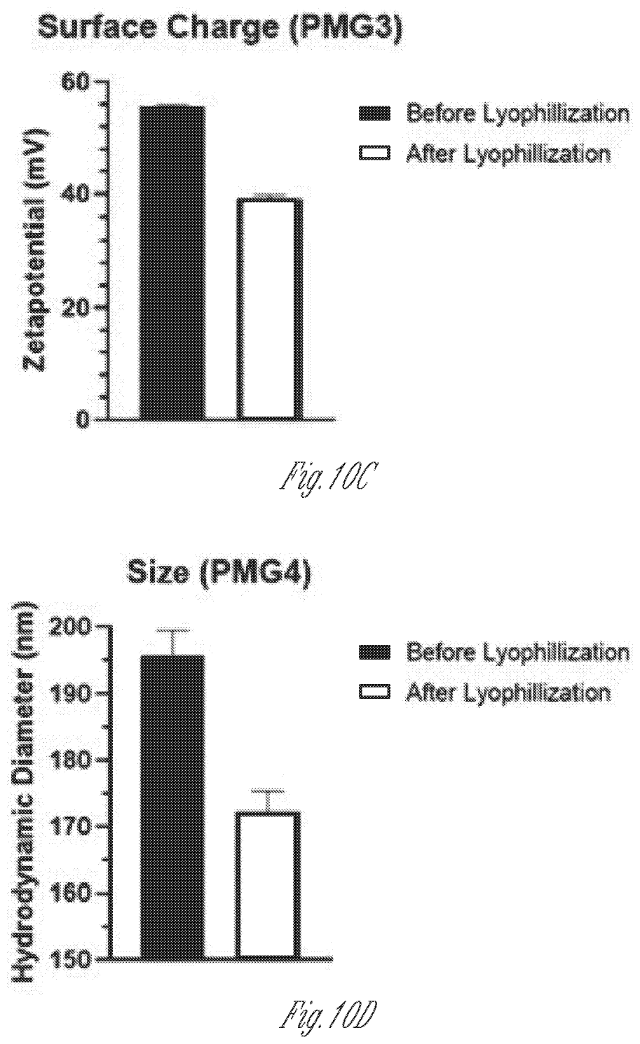

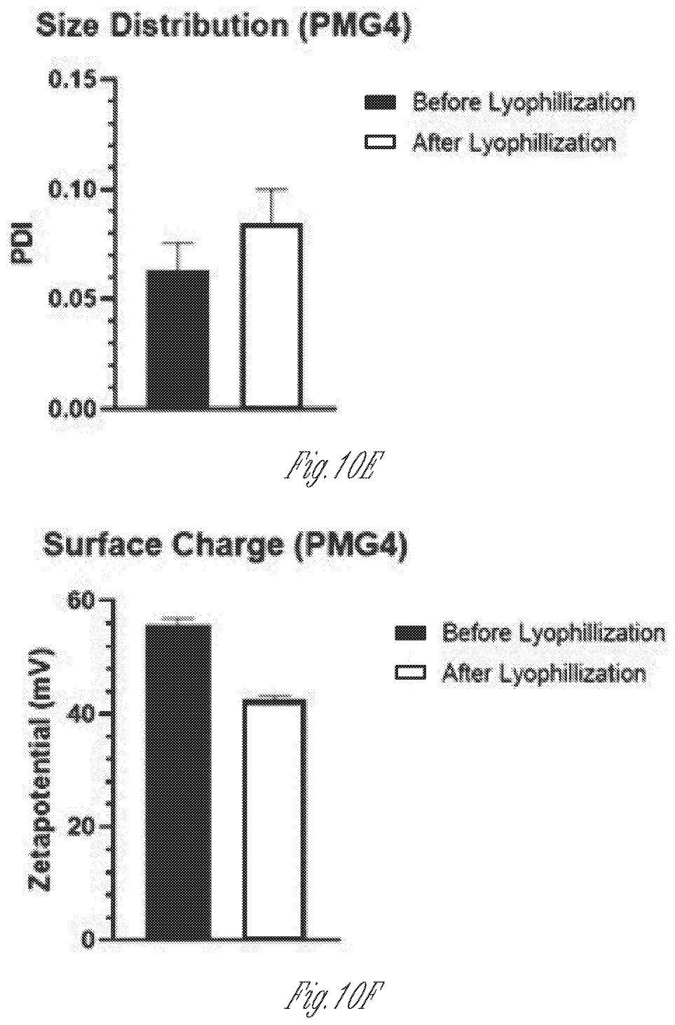

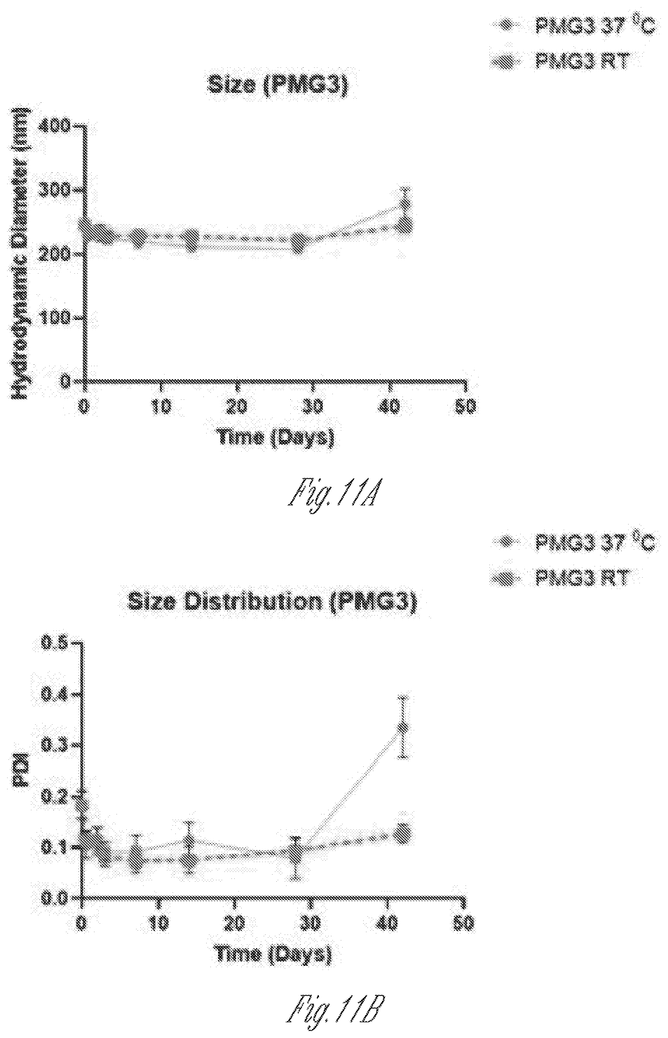

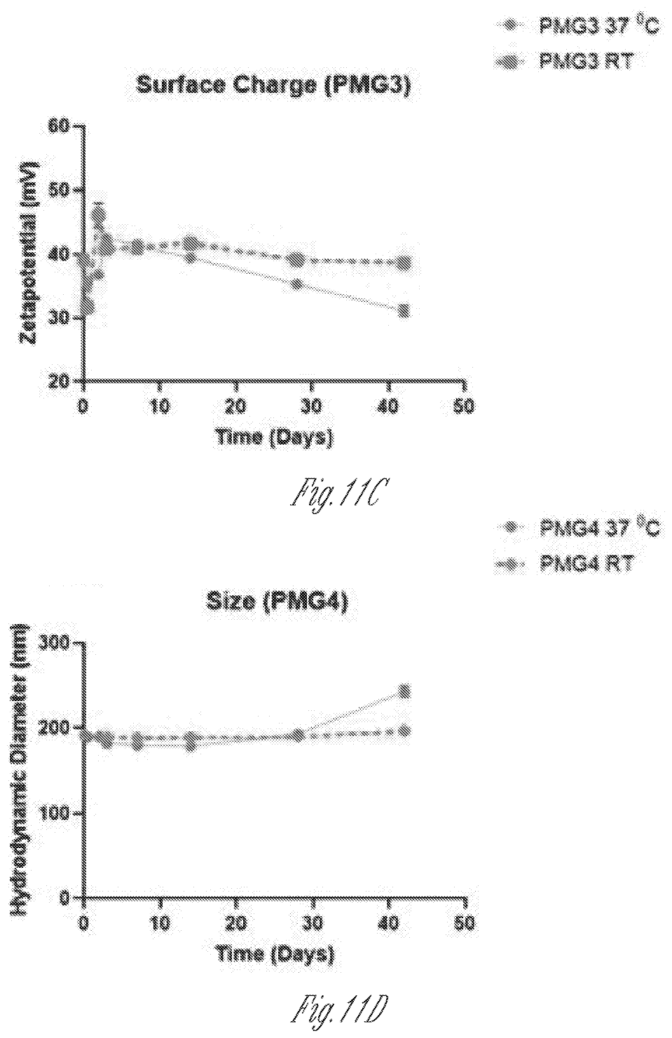

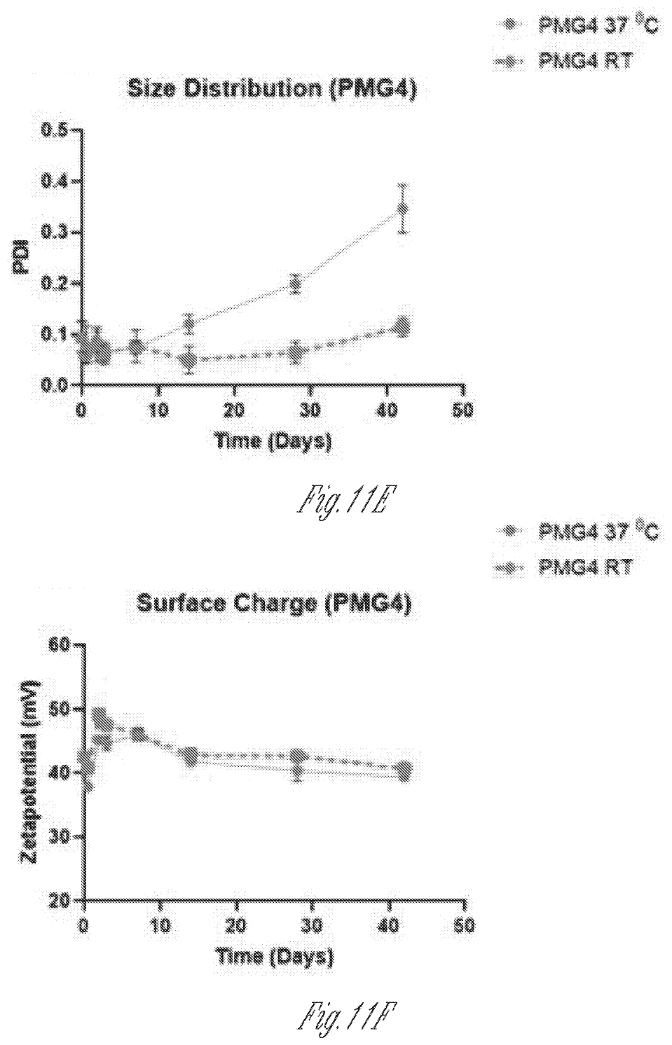

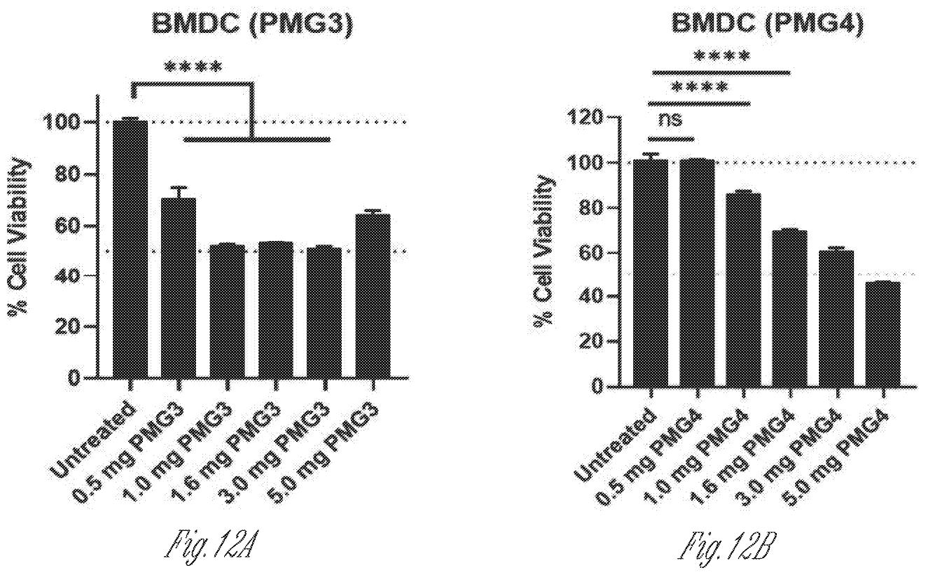

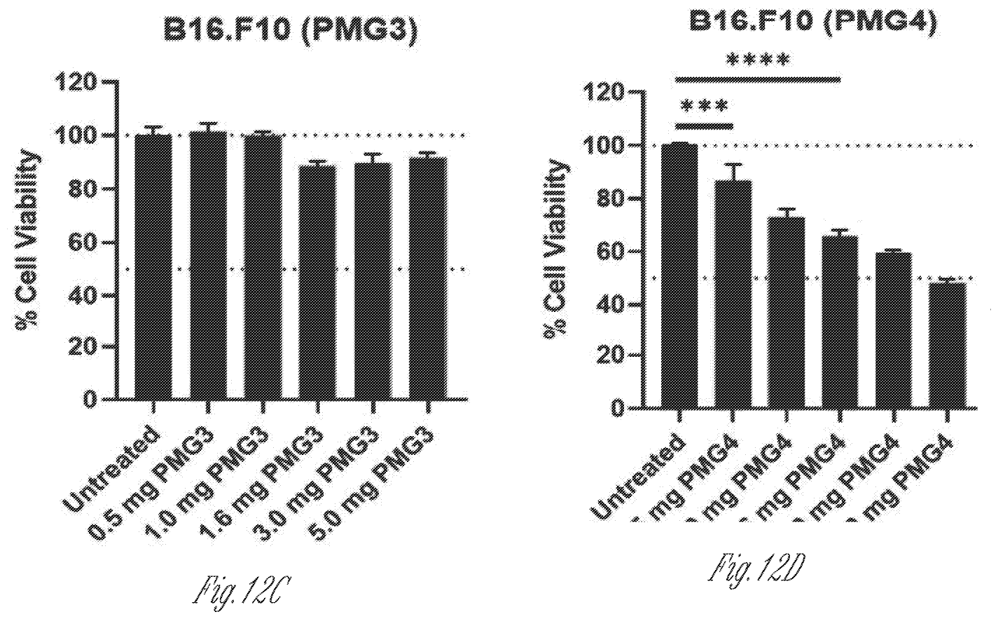

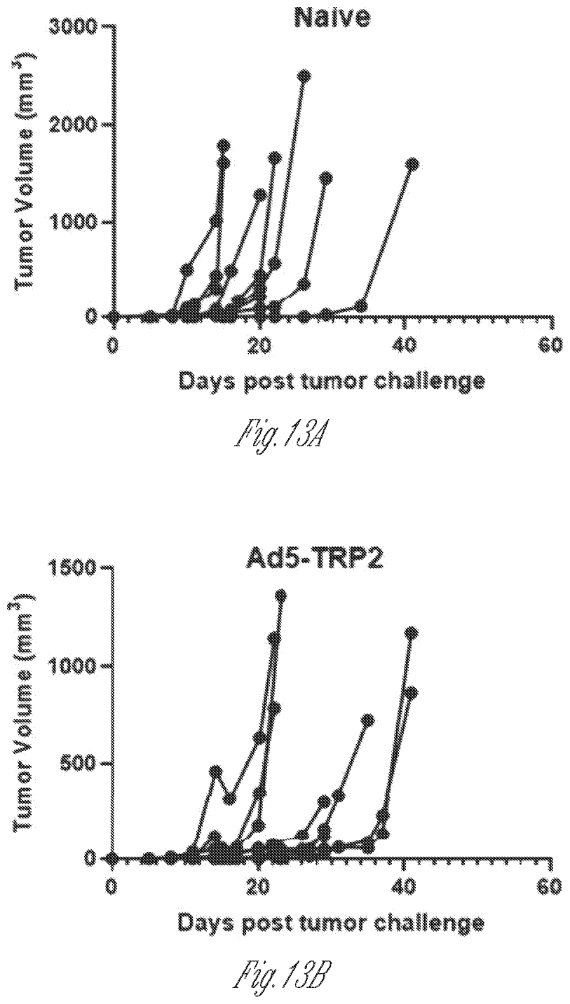

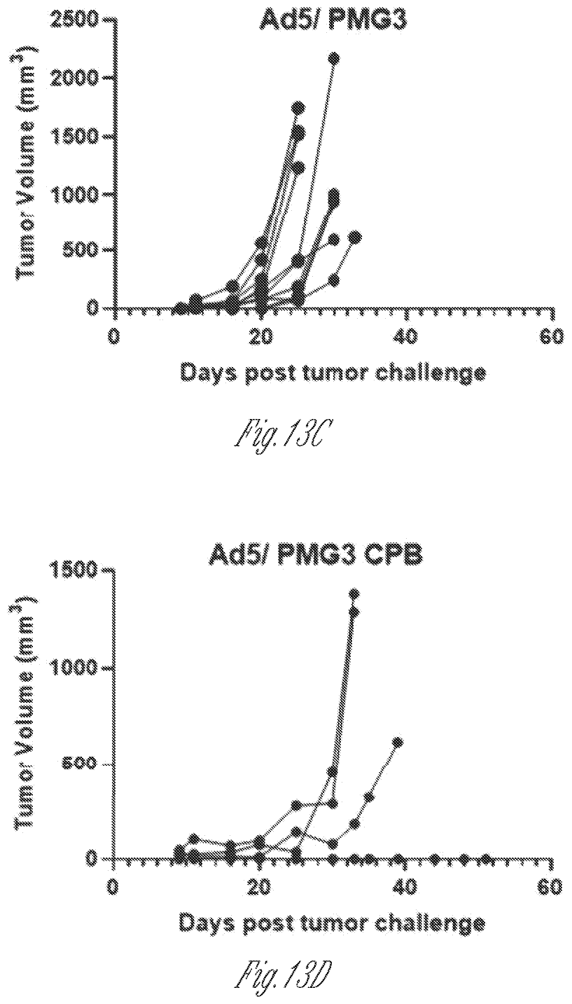

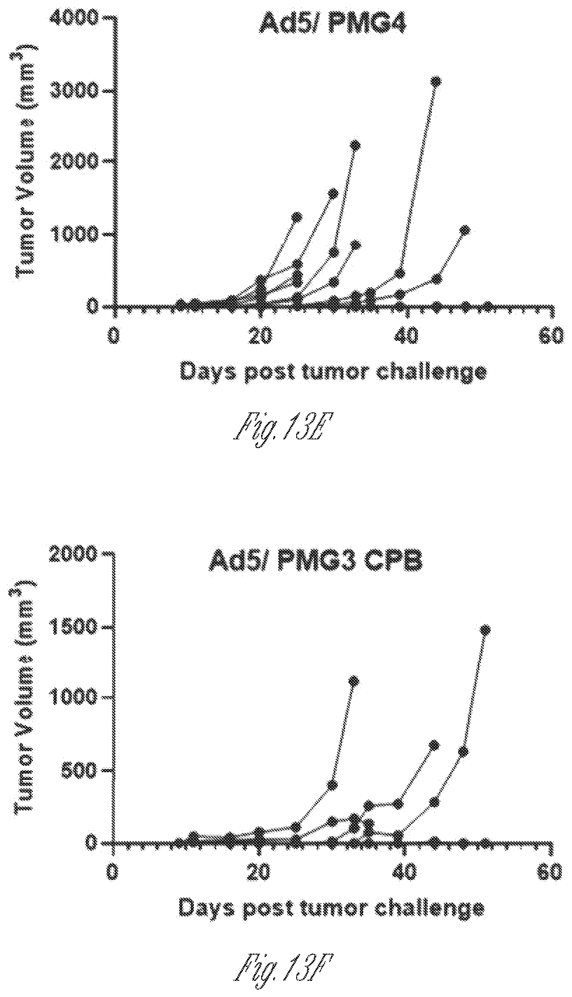

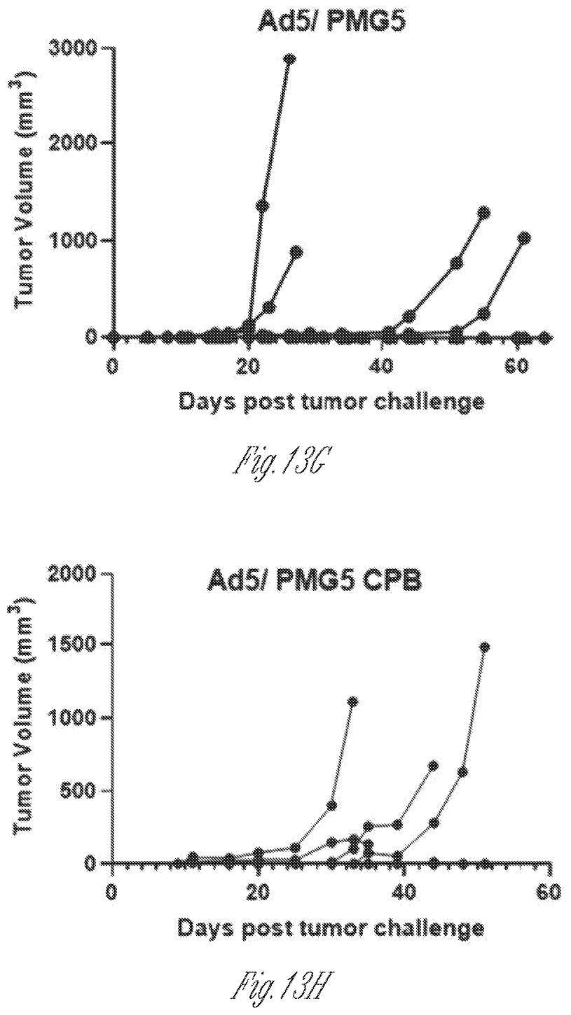

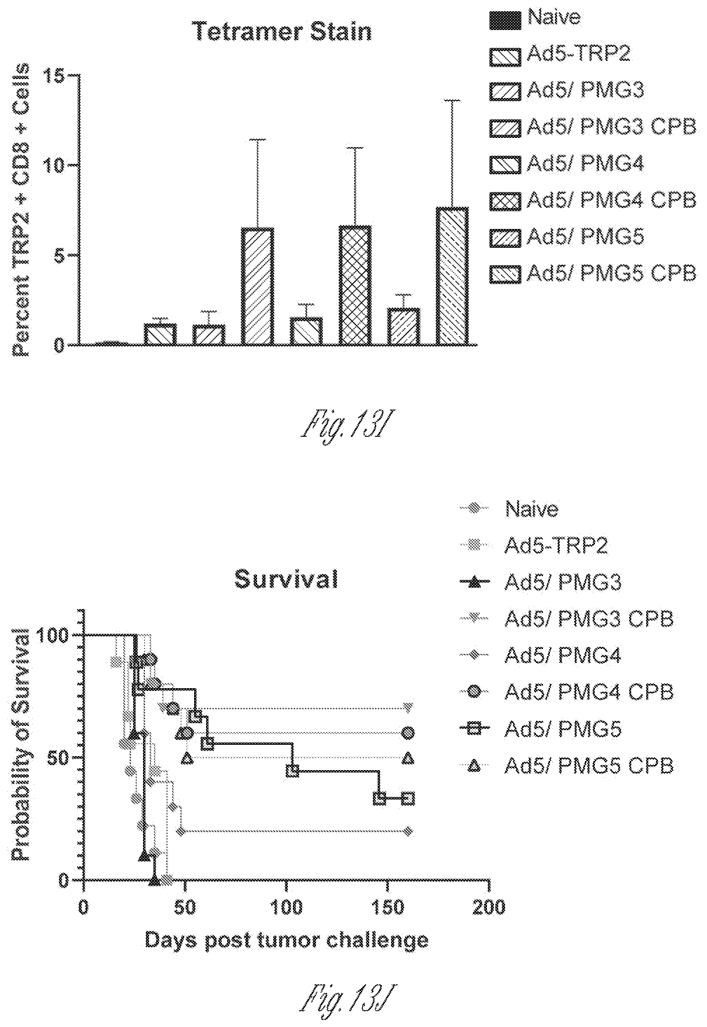

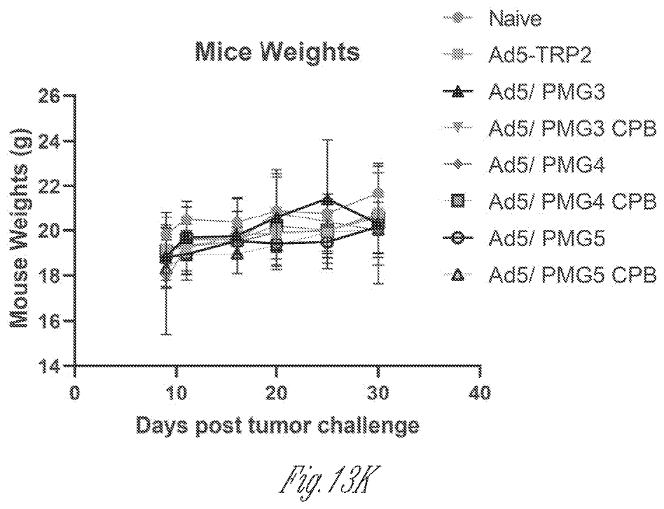

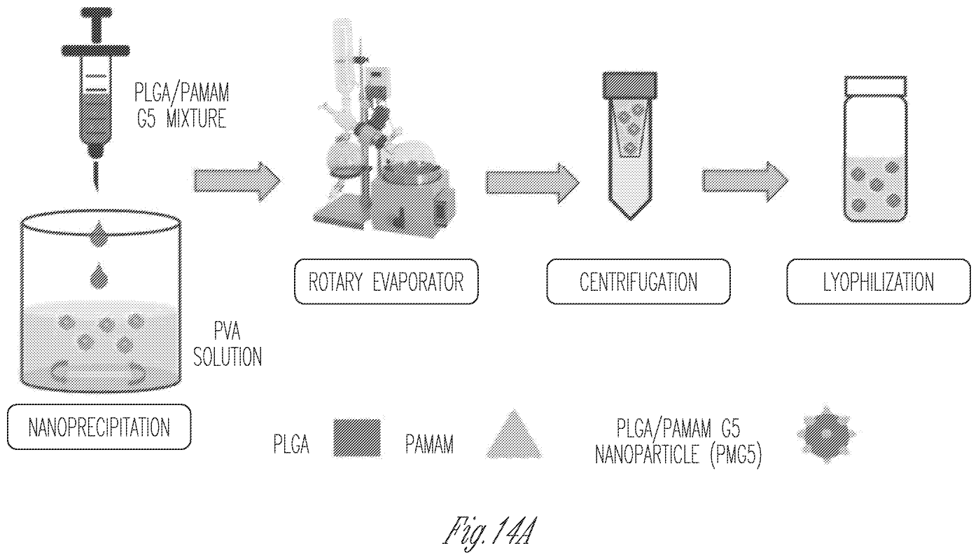

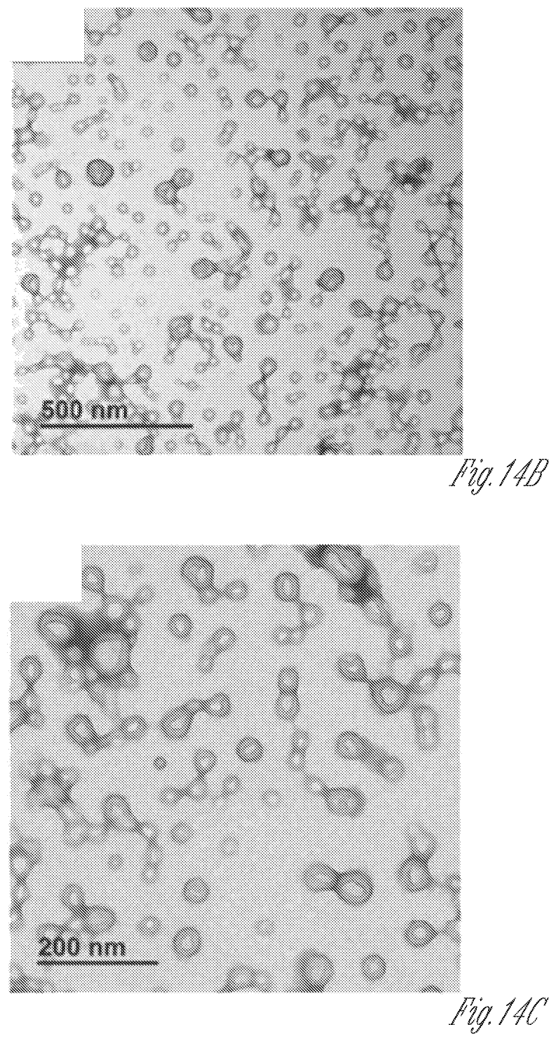

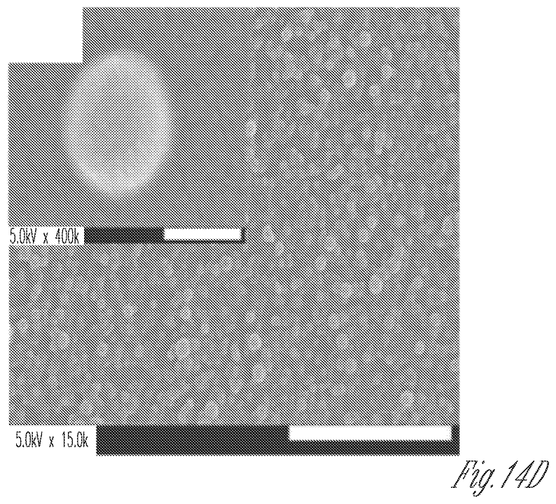

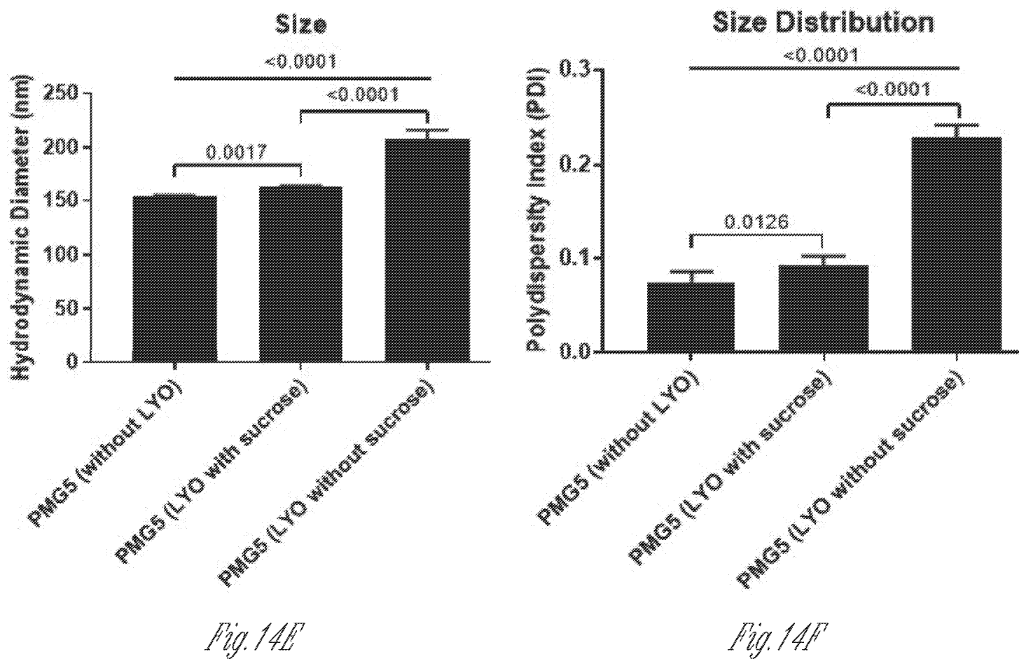

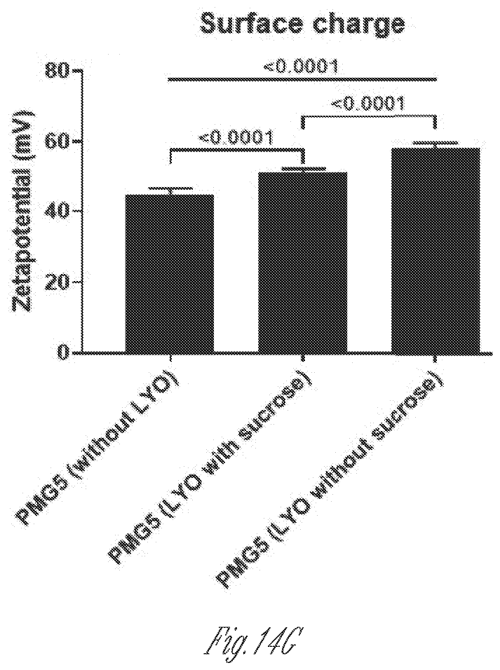

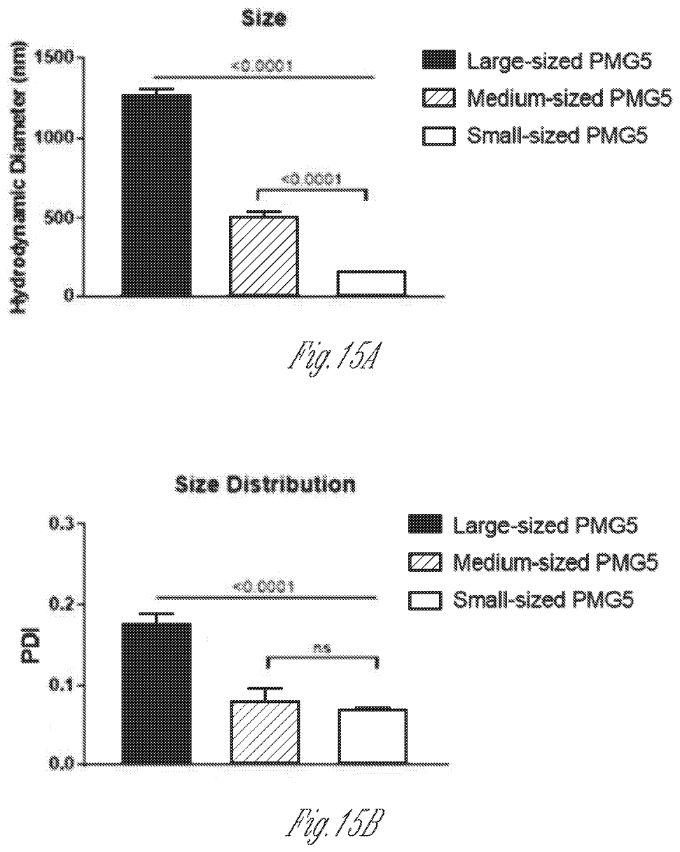

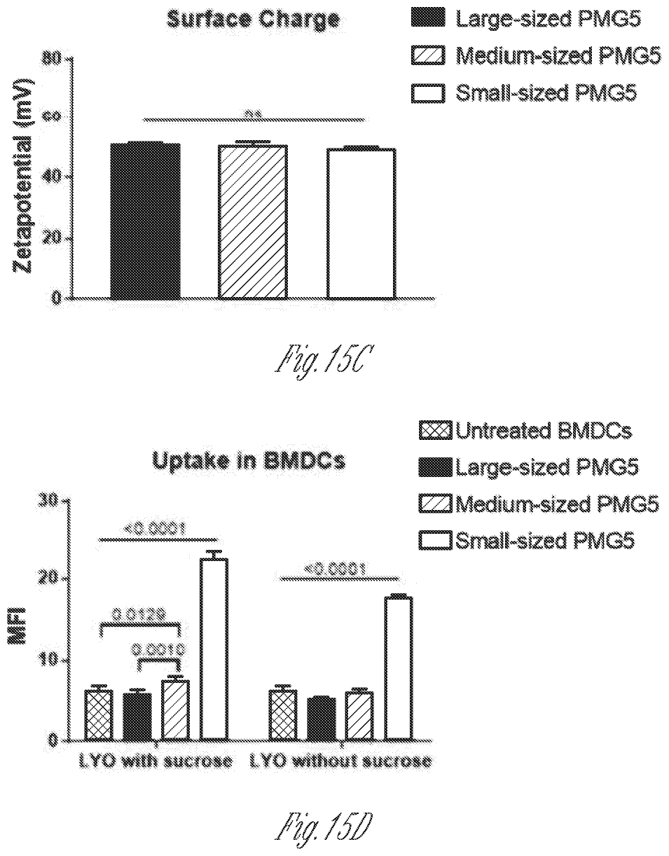

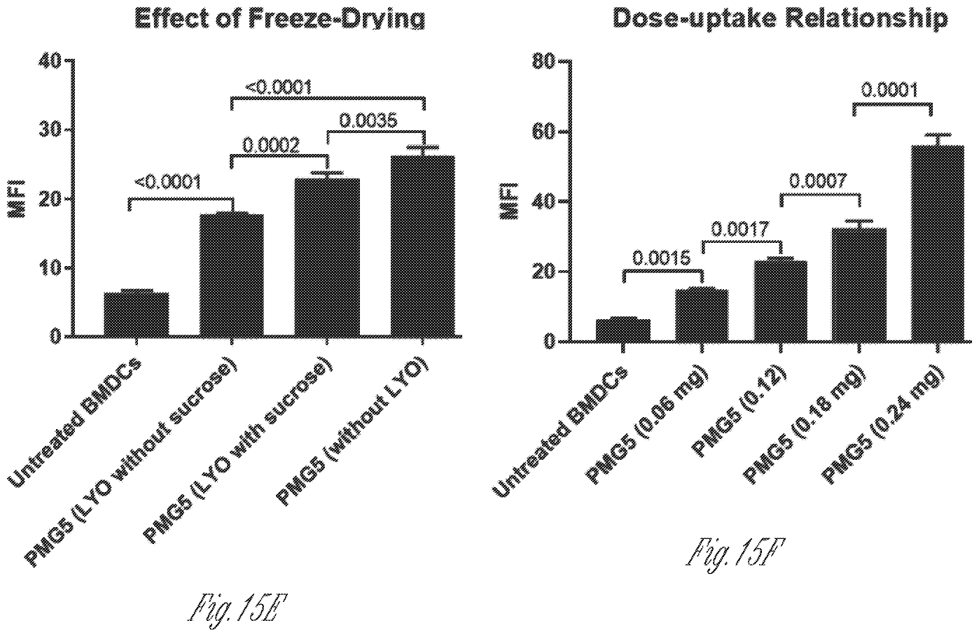

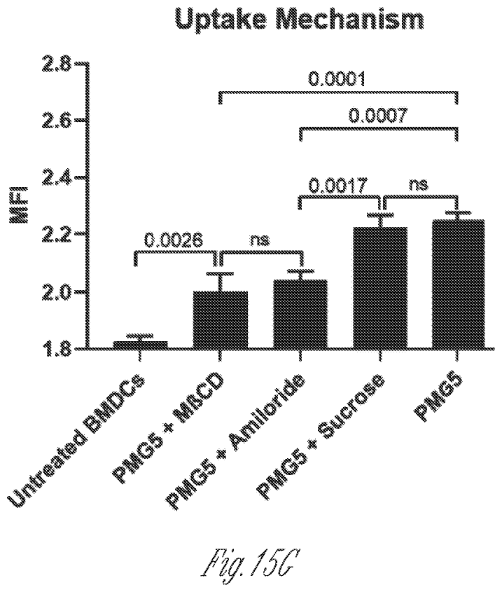

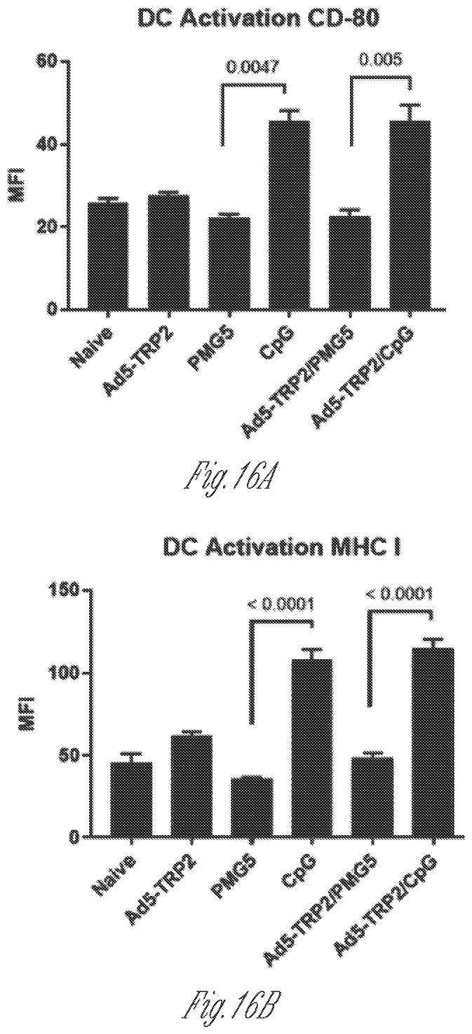

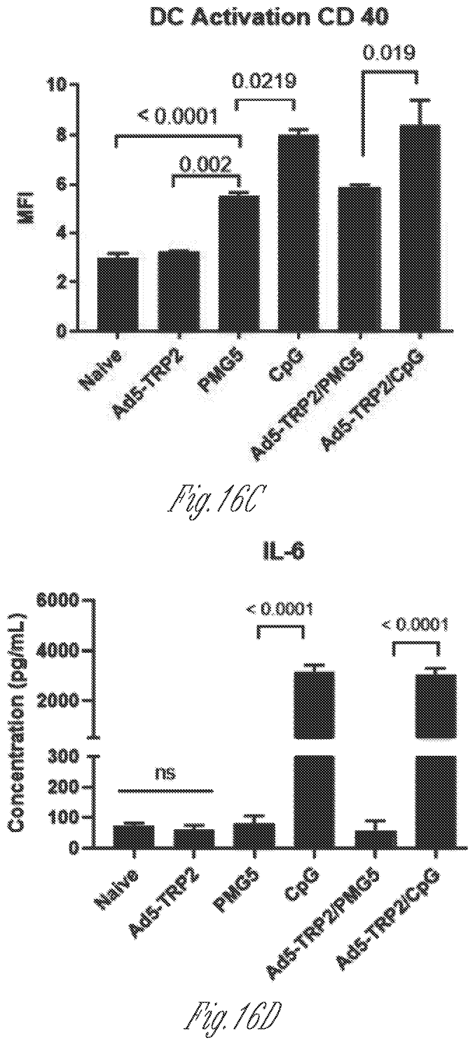

To evaluate the ability of PMG5 to complex with CpG, increasing amounts of CpG were added to a set amount of particles, 1 mg of PMG5, and the ZP recorded with the aim of achieving maximal CpG loading and negligible soluble CpG remaining. The concentration where there was a change in ZP from negative to positive was taken as concentration at which CpG B complexed completely with PMG5. FIG. 6A shows that the zetapotential of the particles was slightly positive/almost neutral when 31.25 μg CpG was added suggesting the approaching of saturation. This was further confirmed when these complexes were run on a gel (showing no free CpG present) FIG. 6B; whilst doubling the concentration of CpG (62.5 ug) resulted in a further decrease in ZP and excess soluble CpG remaining, indicating saturation was achieved. Unmodified amine terminated PAMAM is known to be cytotoxic in vitro and in vivo (Araujo et al., 2018; Sadekar & Ghandehari, 2012; Thiagarajan et al., 2013). To evaluate if the PMG5 formulation was toxic in the quantities necessary to complex with CpG, an MTS assay (viability assay) was performed on B16F10 (melanoma) cells and BMDCs (primary murine cells) by pretreating with varying amounts of PMG5 particles. The results in FIGS. 7A-B, demonstrate that PMG5 did not induce significant cytotoxicity in the ranges that were being used to complex CpG B.

### Dendritic Cell (DC) Activation

BMDCs were shown to take up PMG5 (FIG. 4A); however, the effect on the activation of BMDCs is not known. To evaluate this, BMDCs were incubated with PMG5 and cell surface expression of activation markers (CD80, CD40 and MHC-I) was evaluated. FIG. 8B demonstrates that CpG B when complexed with PMG5 (CpG-PMG5) elicits less MHC-I expression whether in the presence of Ad5-TRP2 or not. However, the expression of the co-stimulatory molecules CD80 and CD40, followed a different trend where, regardless of the presence or absence of the Ad5-TRP2, there was no difference in the cell surface expression of activation markers when BMDCs were incubated with CpG or CpG-PMG5. Regardless of the marker expression or the presence of Ad5-TRP2, PMG5 elicited less costimulatory molecule expression than CpG B or CpG-PMG5. Evaluating the chemokine/cytokine secretion of BMDCs, showed that while CpG B on its own or complexed with PMG5 elicits both high IL-6 (Th2) and IP-10 (Th1) secretion, in the presence of Ad5-TRP2, PMG5 causes less secretion of IL-6 and IP-10 than CpG or CpG-PMG5. Examining the Th1 vs Th2 ratio (FIG. 8F) Ad5/PMG5 had a significantly higher Th1/Th2 ratio than Ad5/CpG B.

### Antitumor Efficacy

Combining Ad5-TRP2 with PMG5 (Ad5/PMG5) in vivo yielded promising results. FIG. 9K demonstrates that mice vaccinated with Ad5-TRP2 and given subsequent peritumoral administrations of PMG5 (Ad5/PMG5) resulted in an increase in the percent of CD8+ TRP2-specific T cells in the peripheral blood. This treatment group also exhibited significantly reduced tumor growth rates (FIG. 9G) which translated into substantial and significant increases in median survival (103 days for Ad5/PMG5 treated mice) compared to naïve mice (23 days) (FIG. 91). Mice that were given this treatment were also protected from subsequent tumor challenges 60 days after initial treatment thus demonstrating the generation of adaptive immunity.

Evaluation of PM Made with Generations 3 and 4 PAMAM

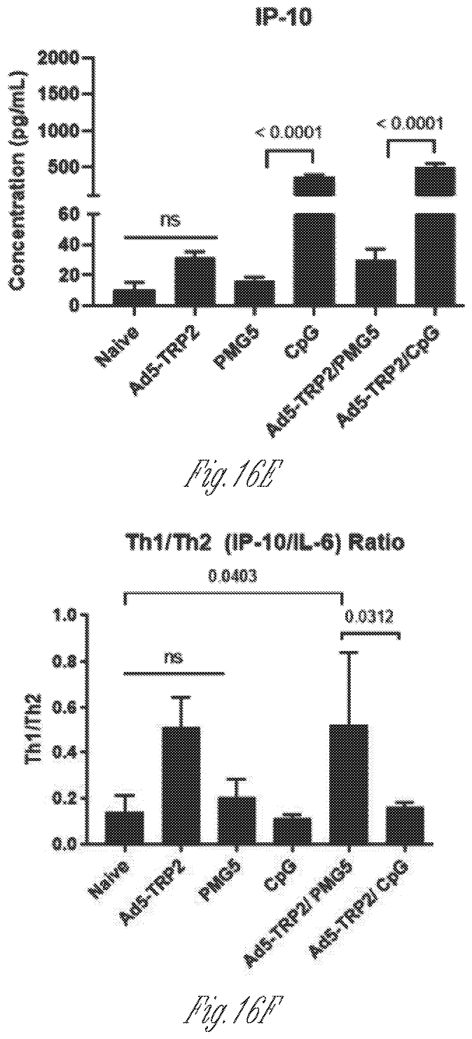

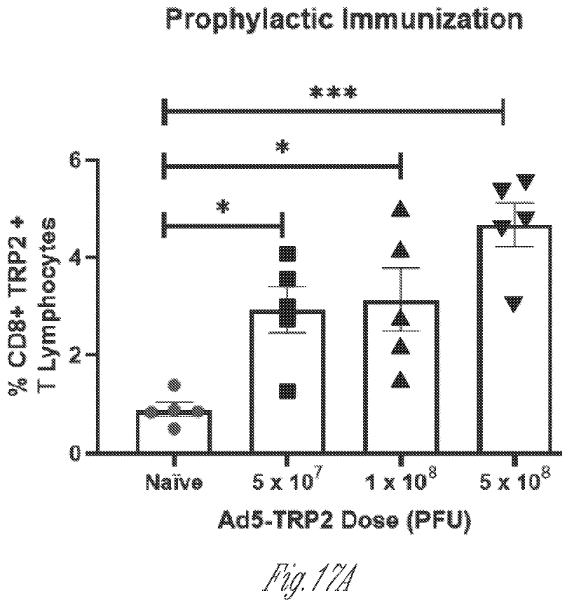

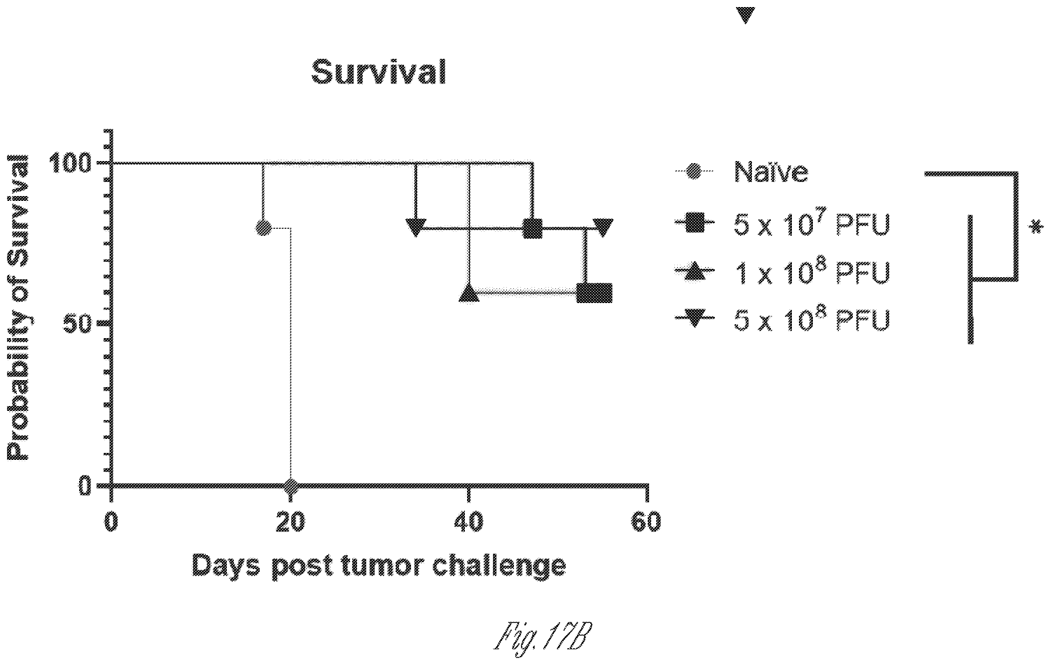

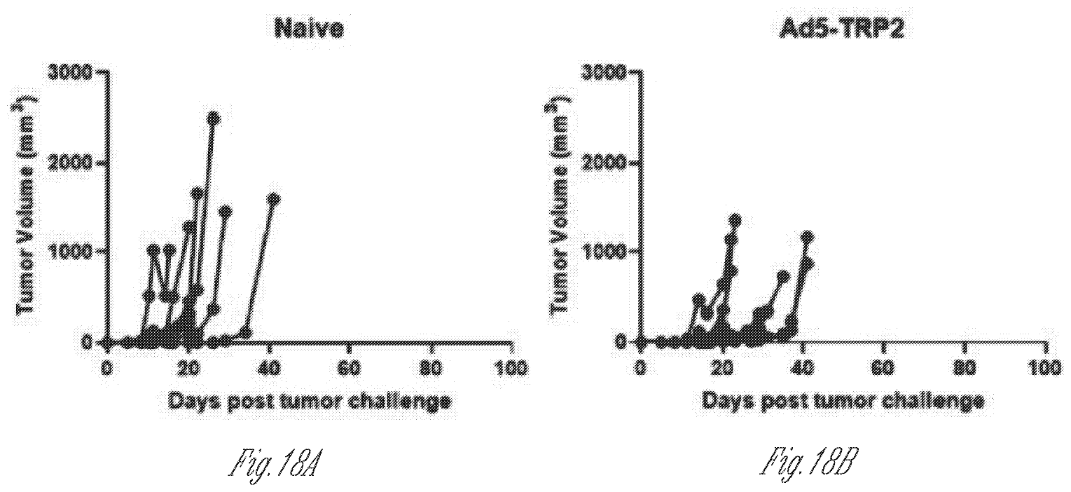

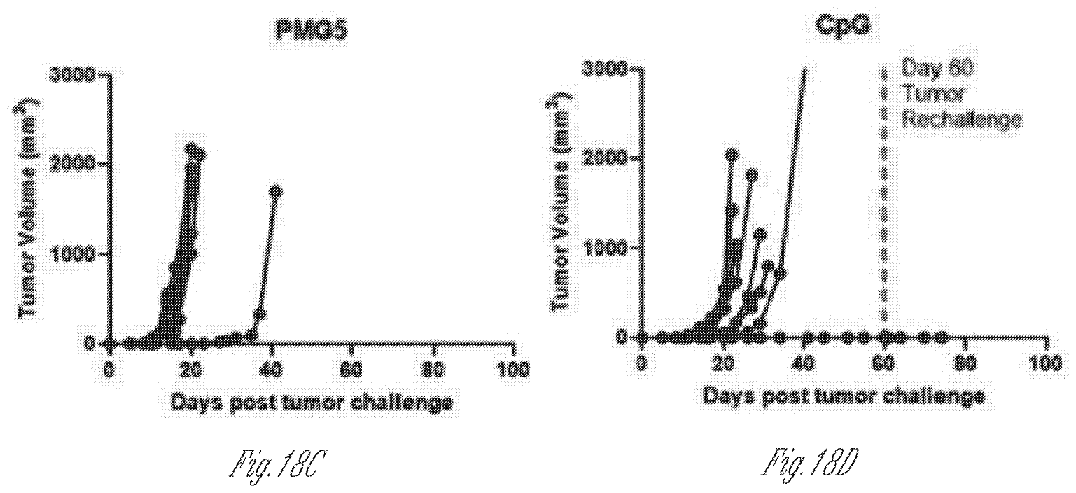

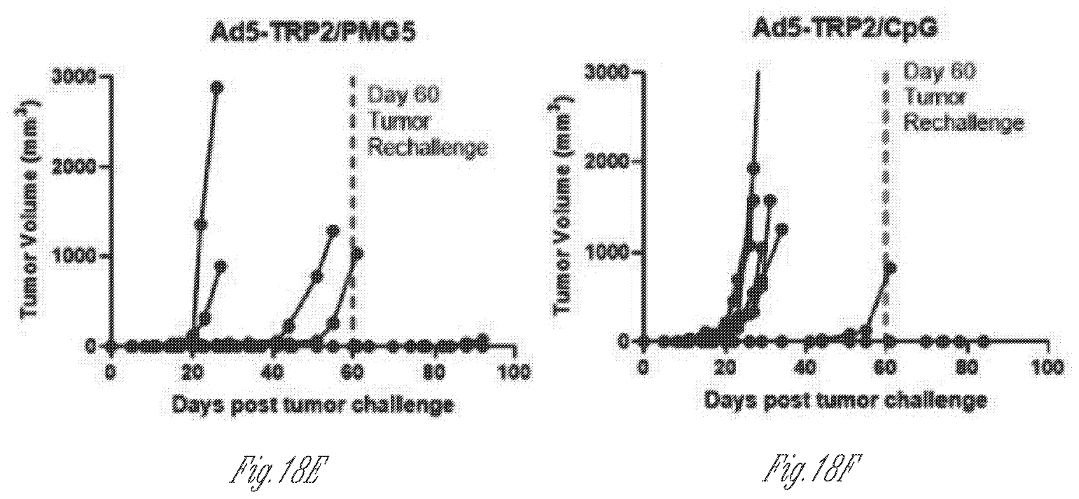

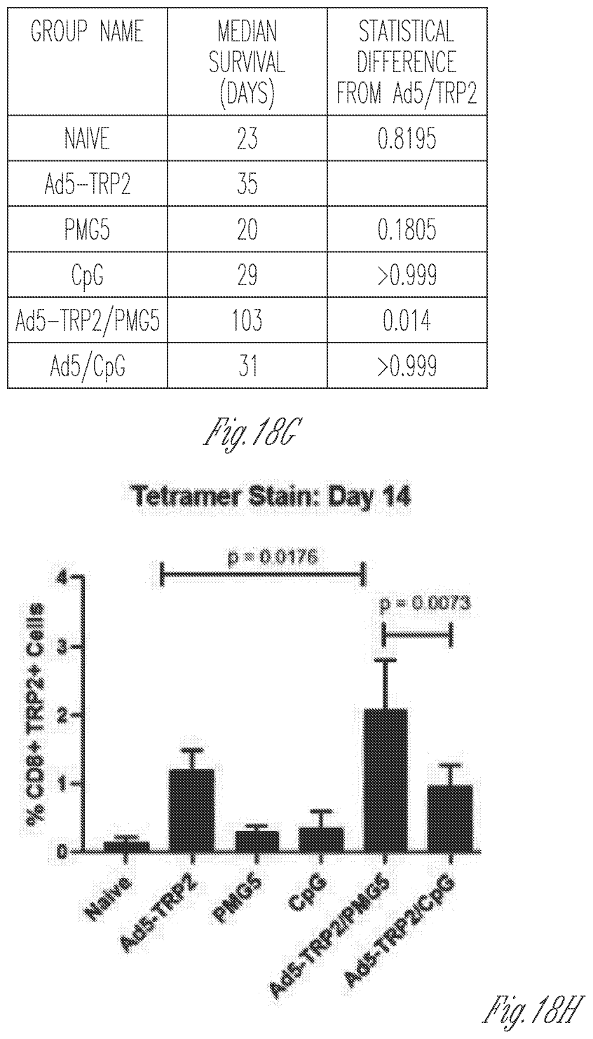

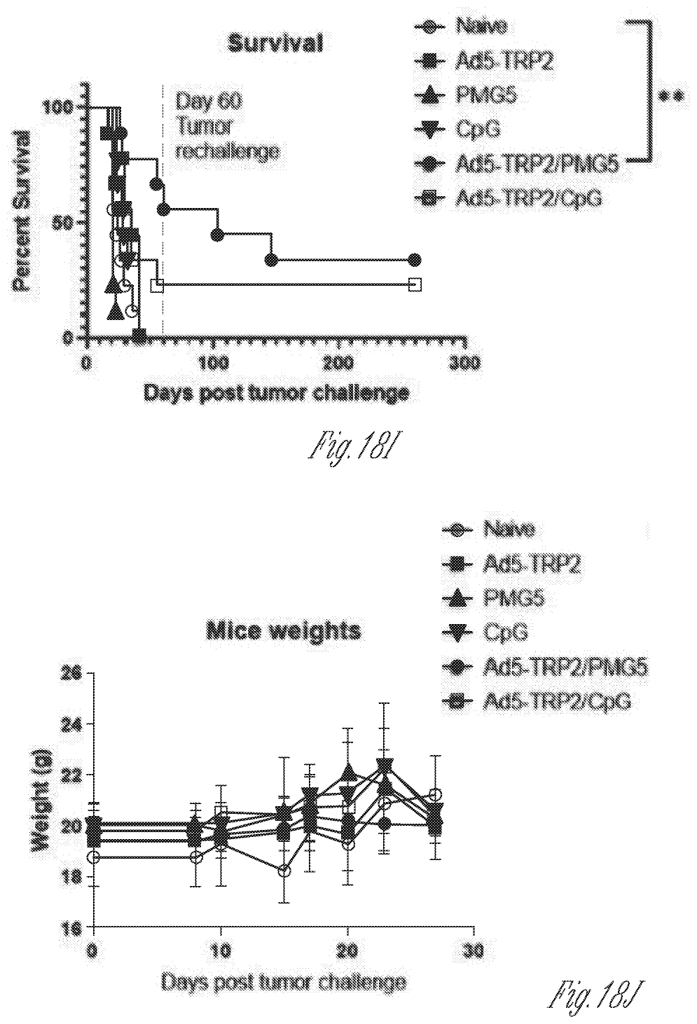

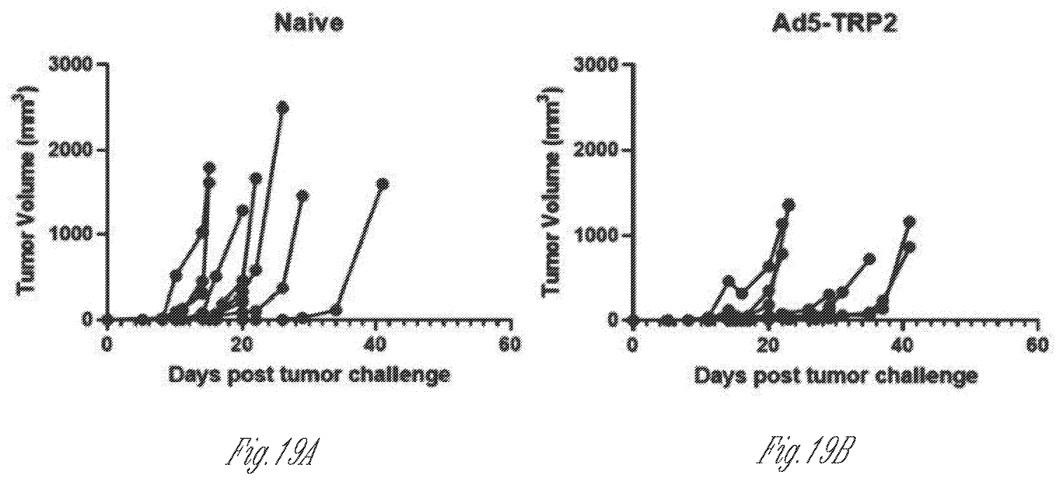

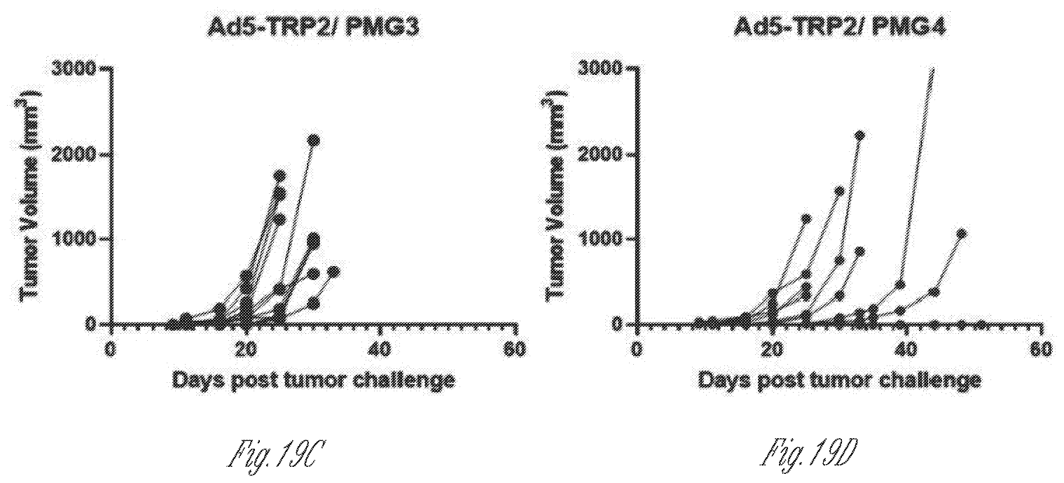

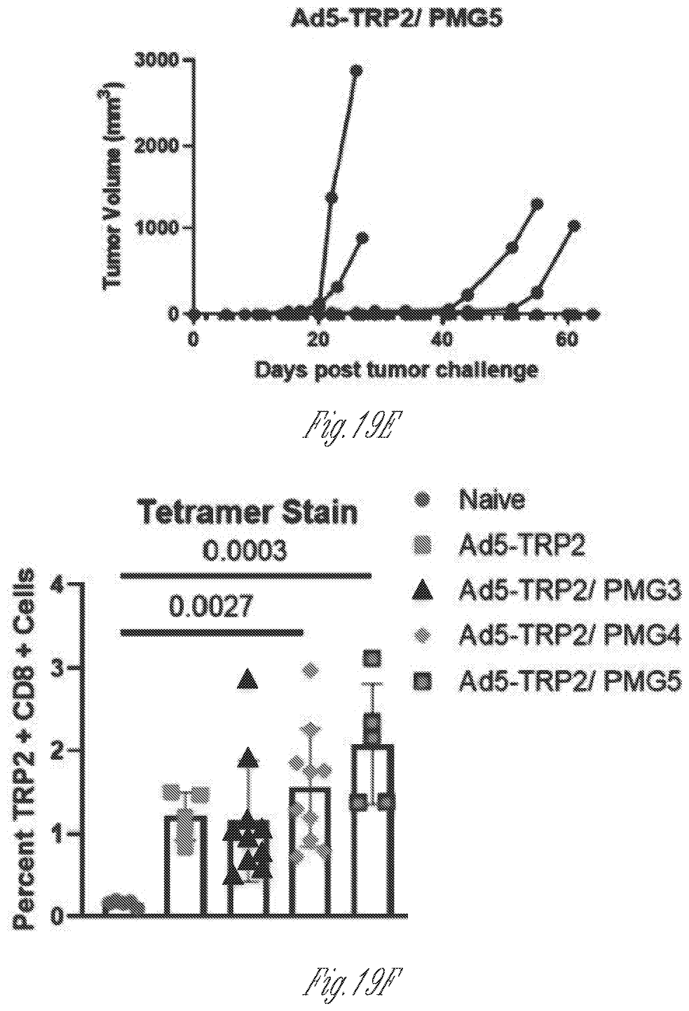

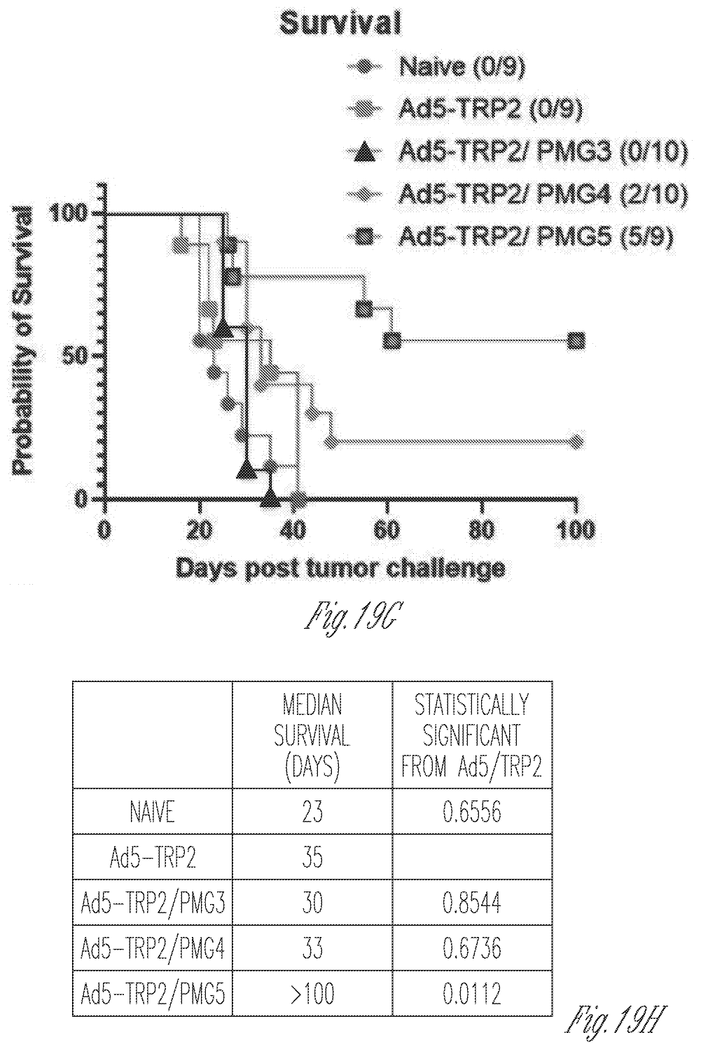

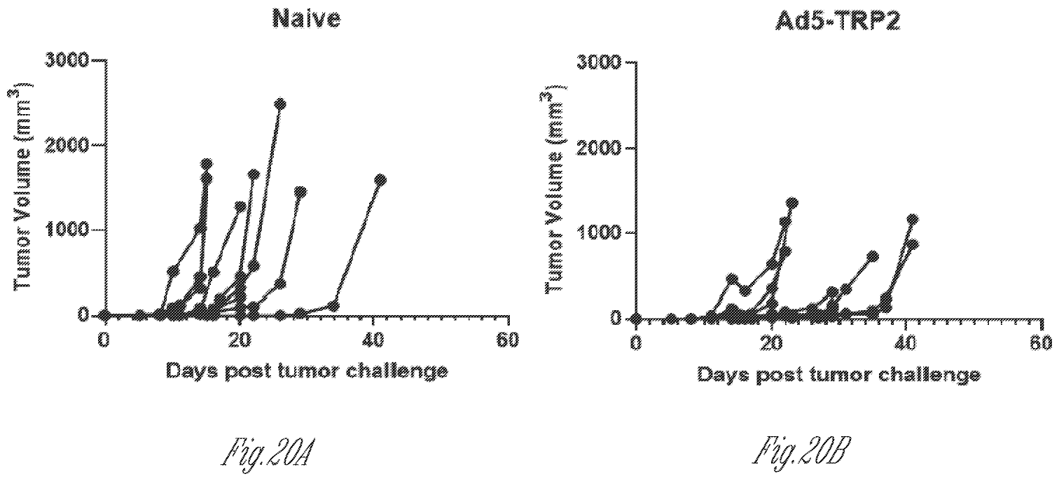

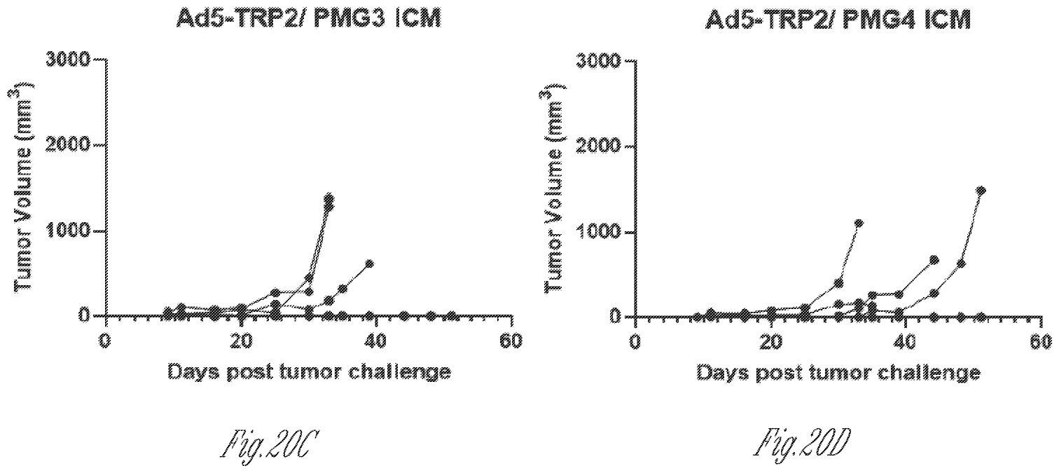

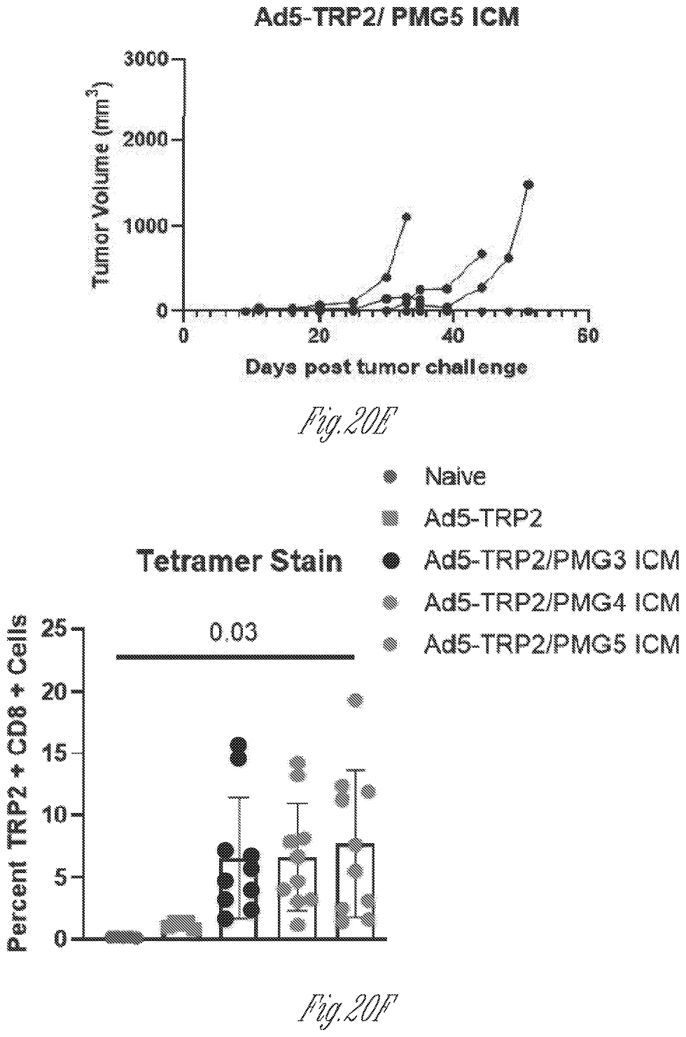

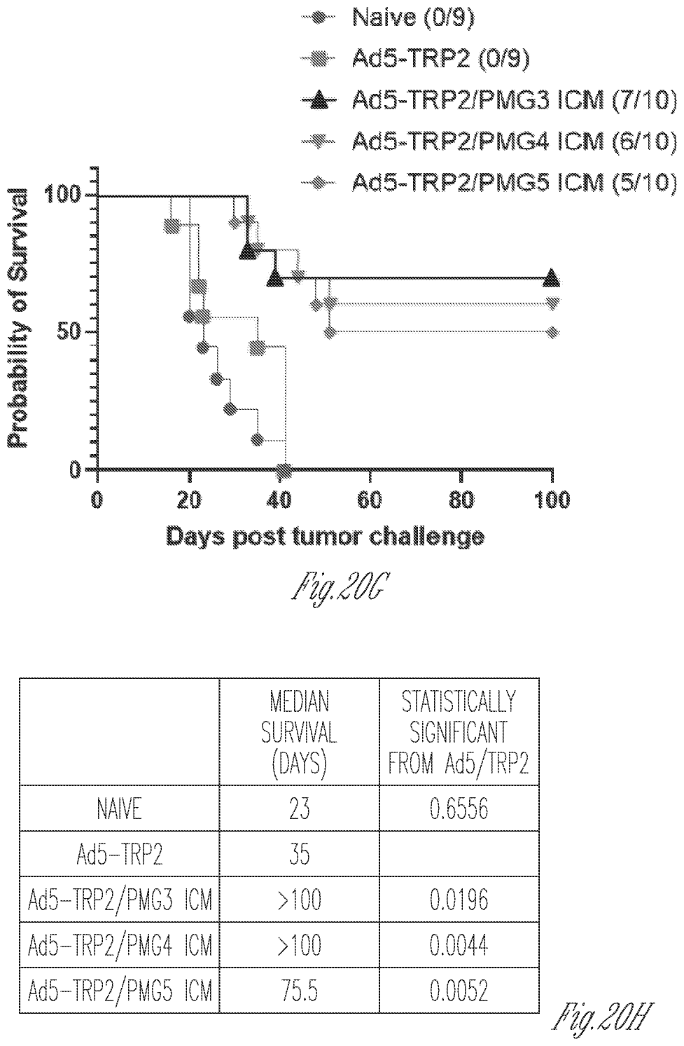

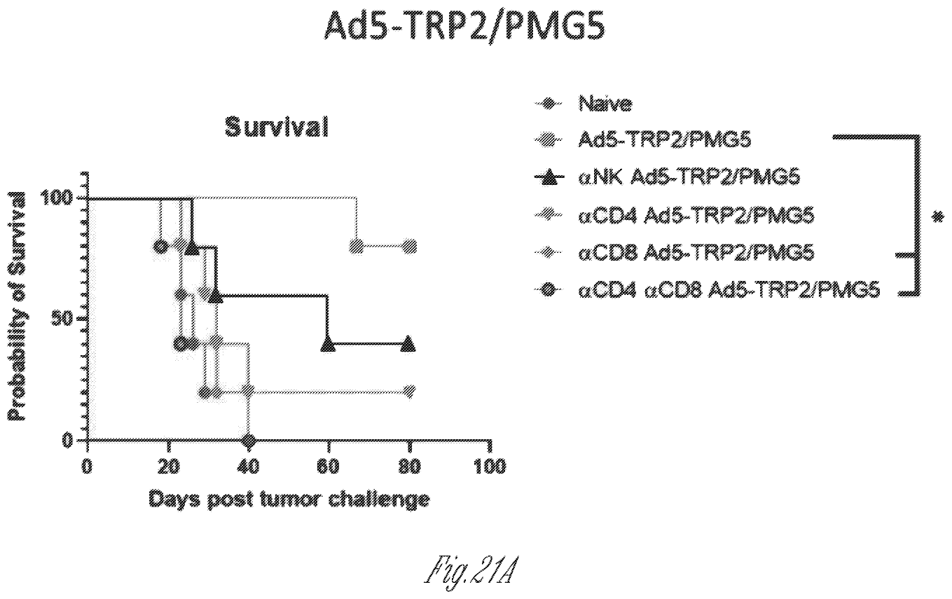

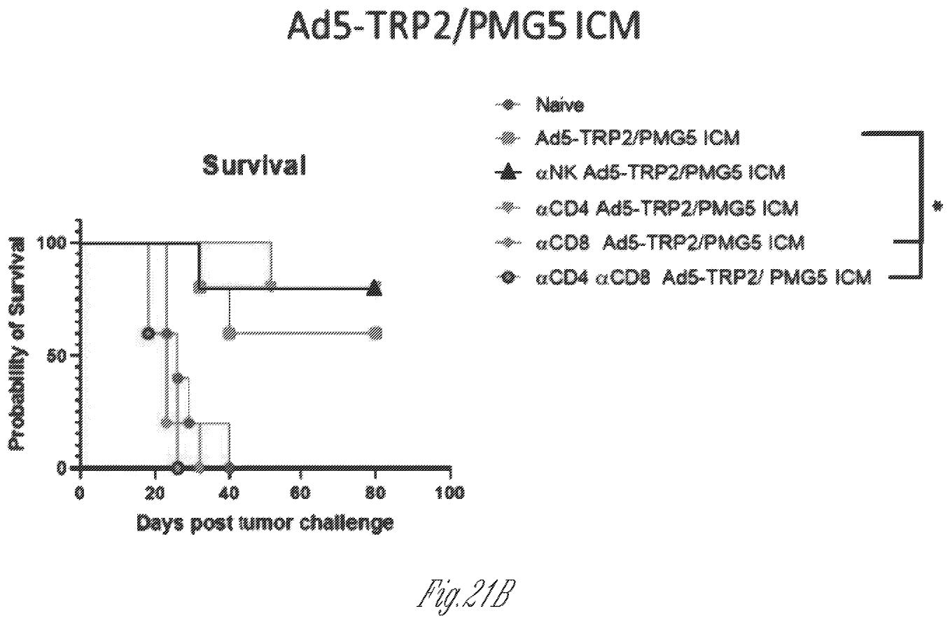

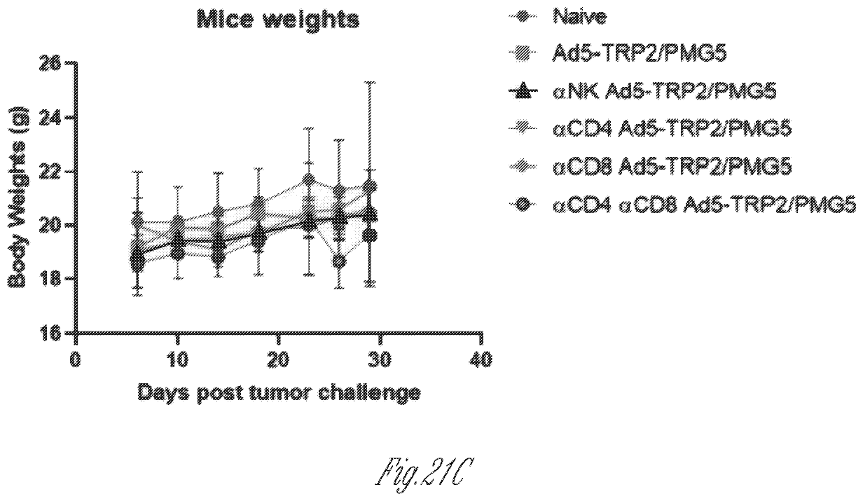

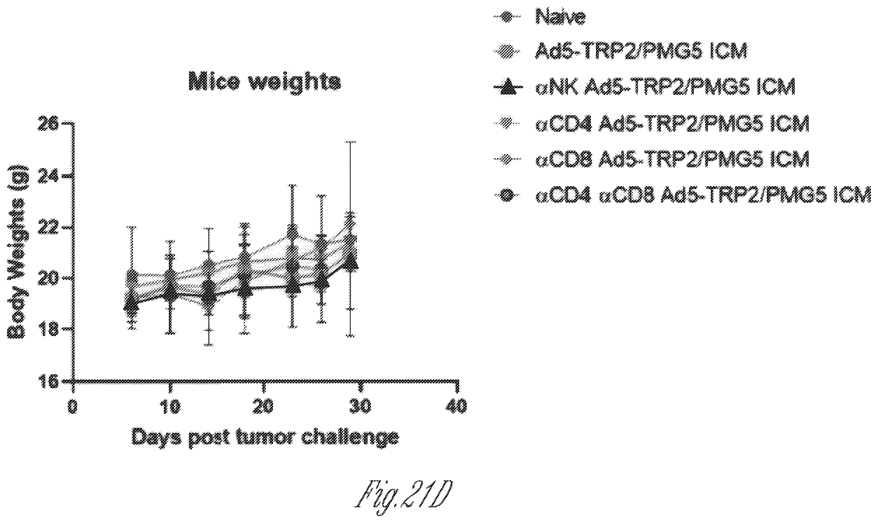

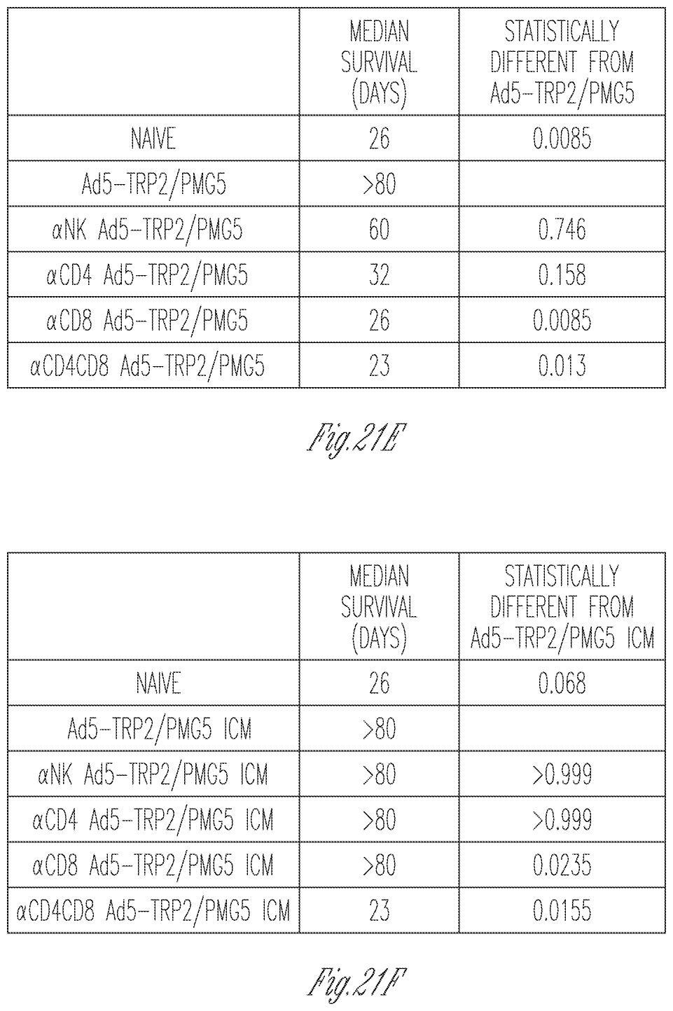

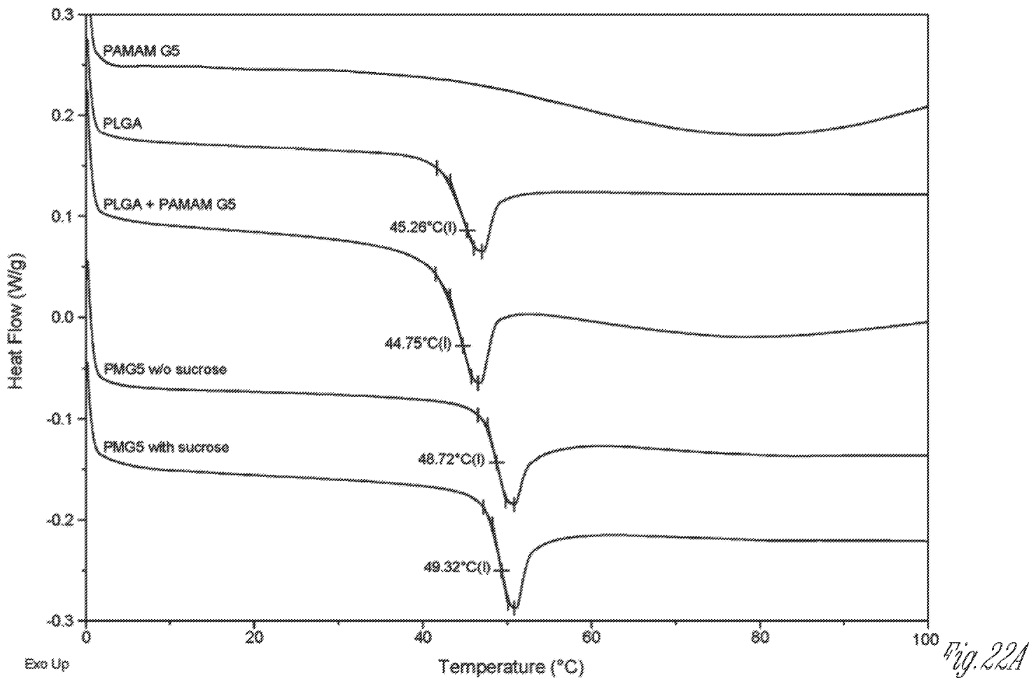

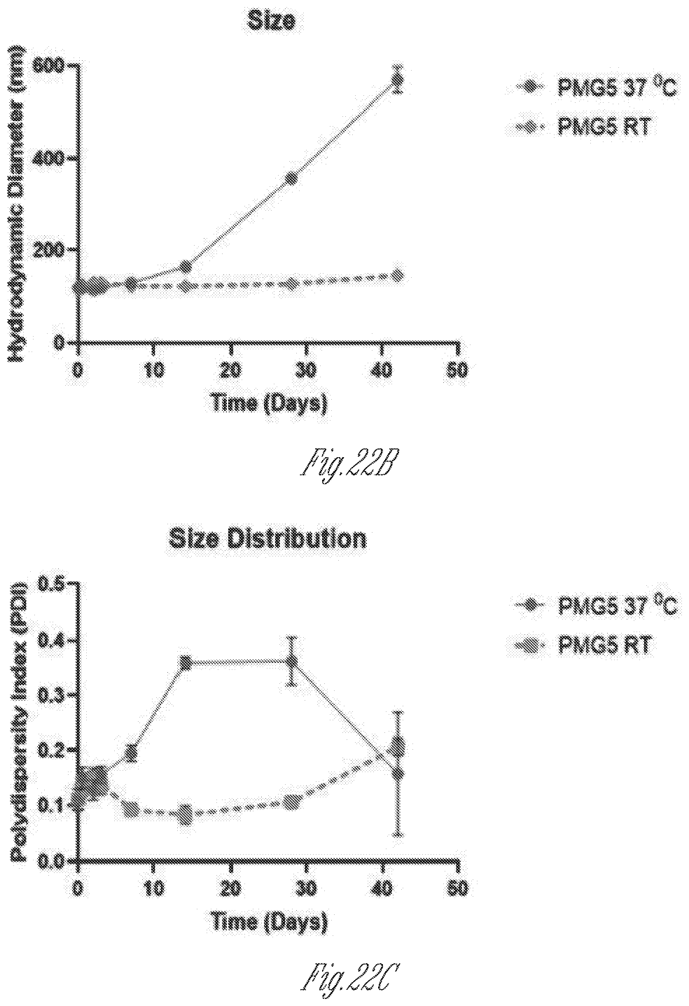

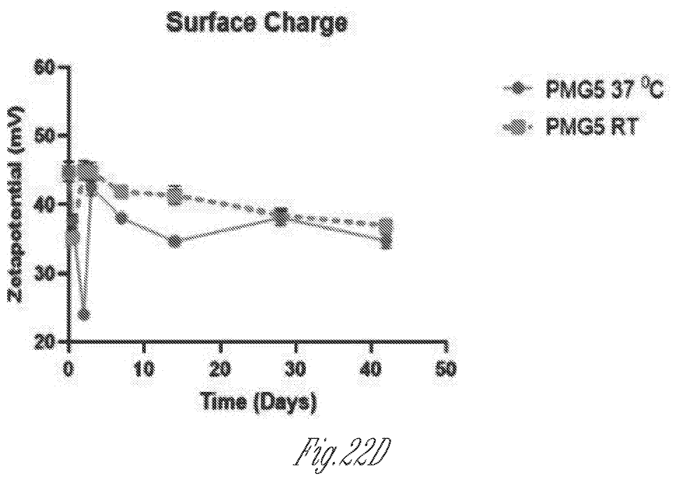

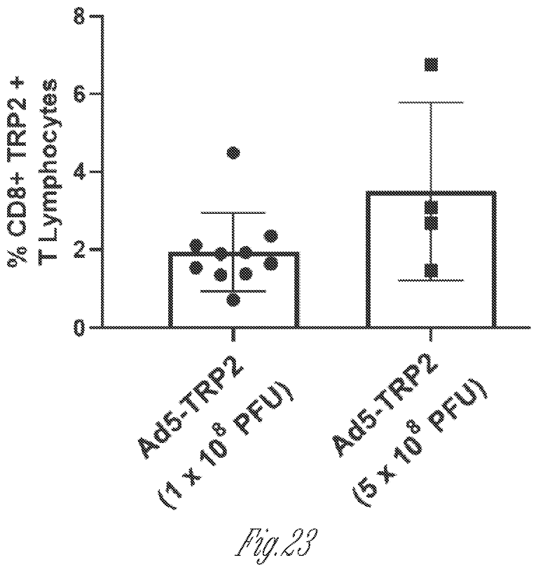

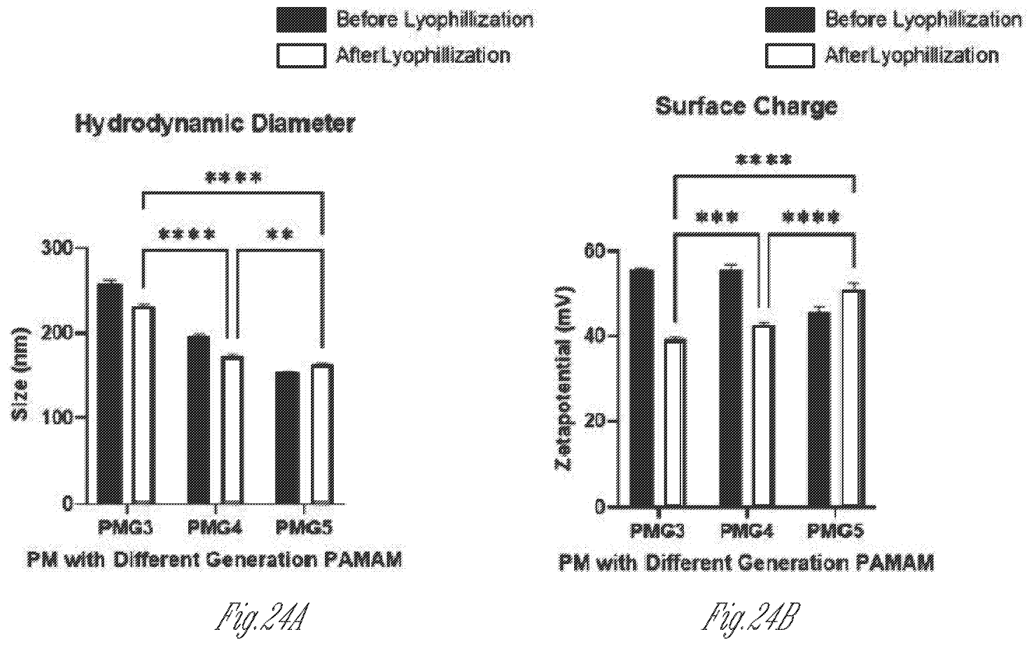

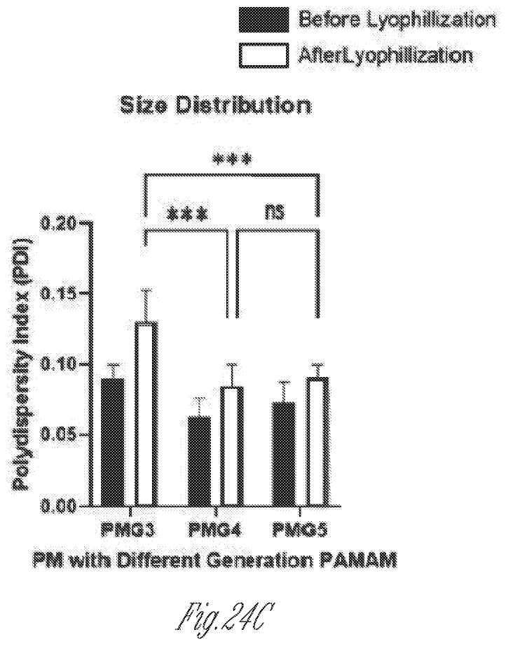

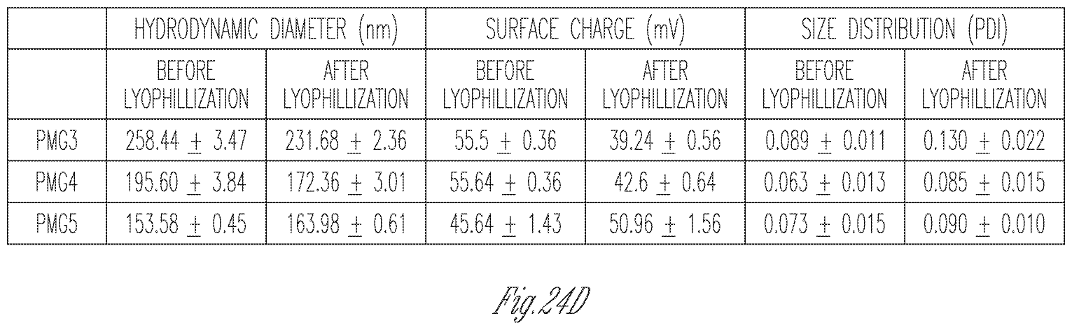

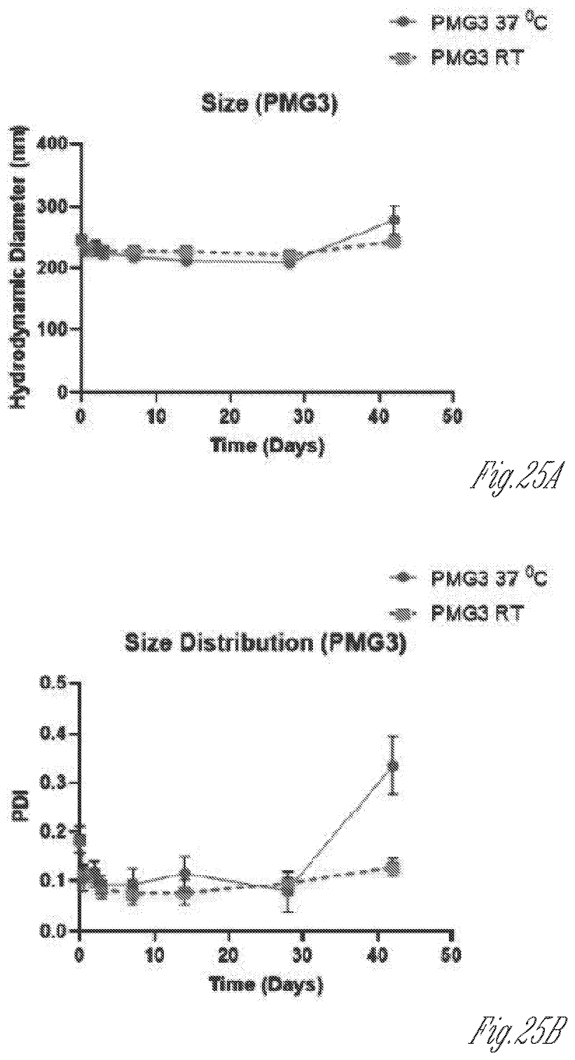

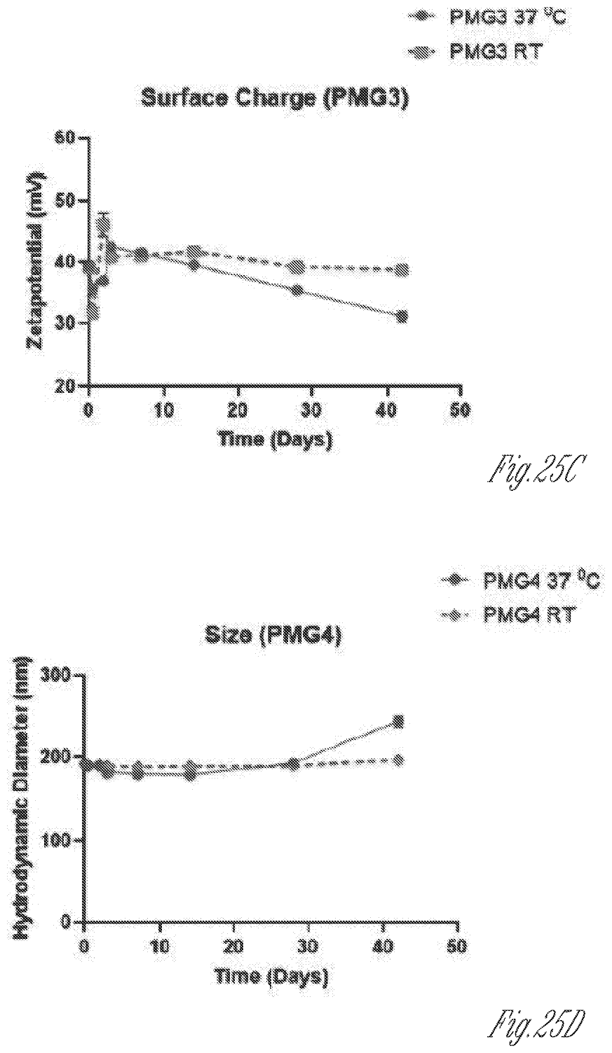

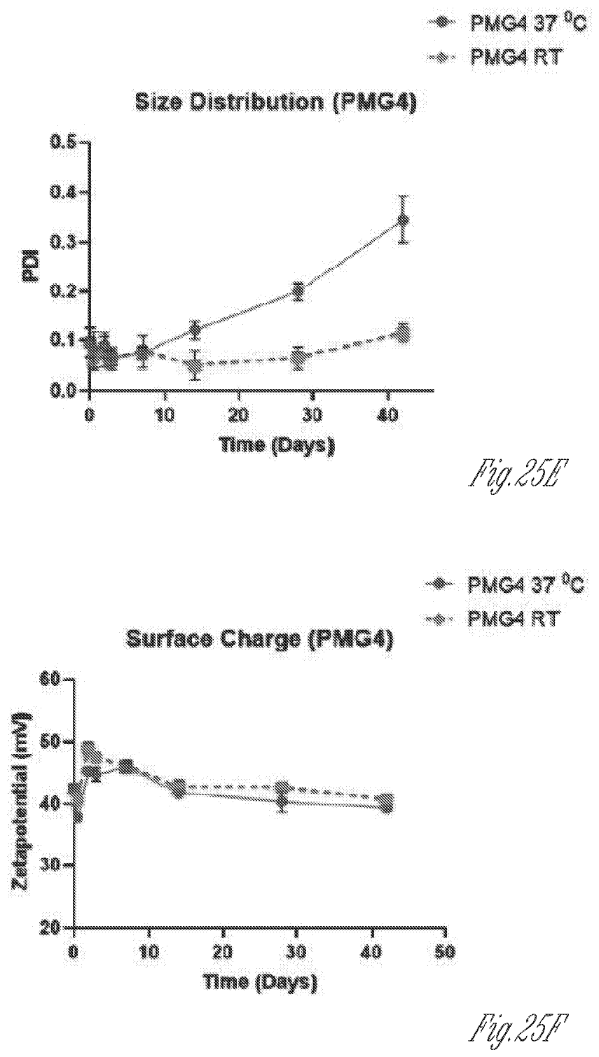

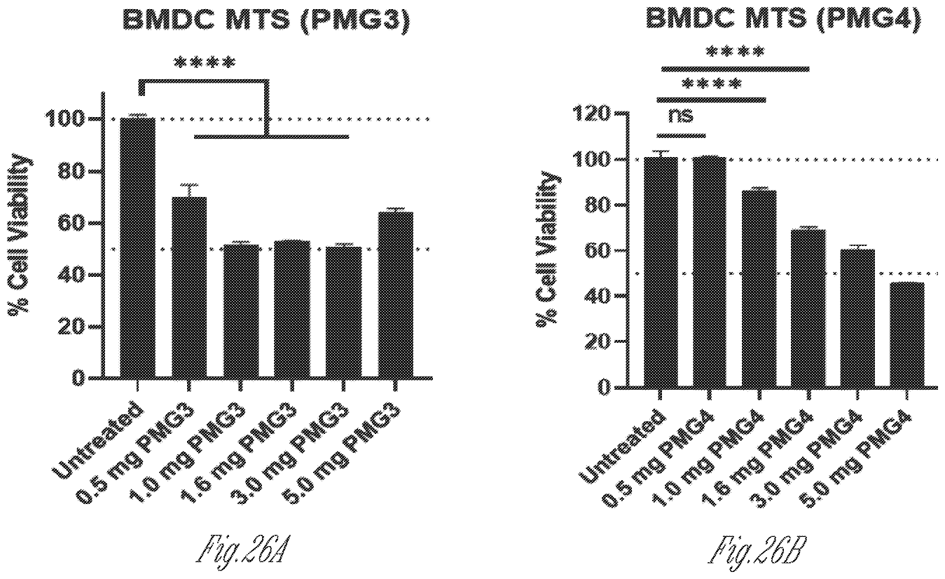

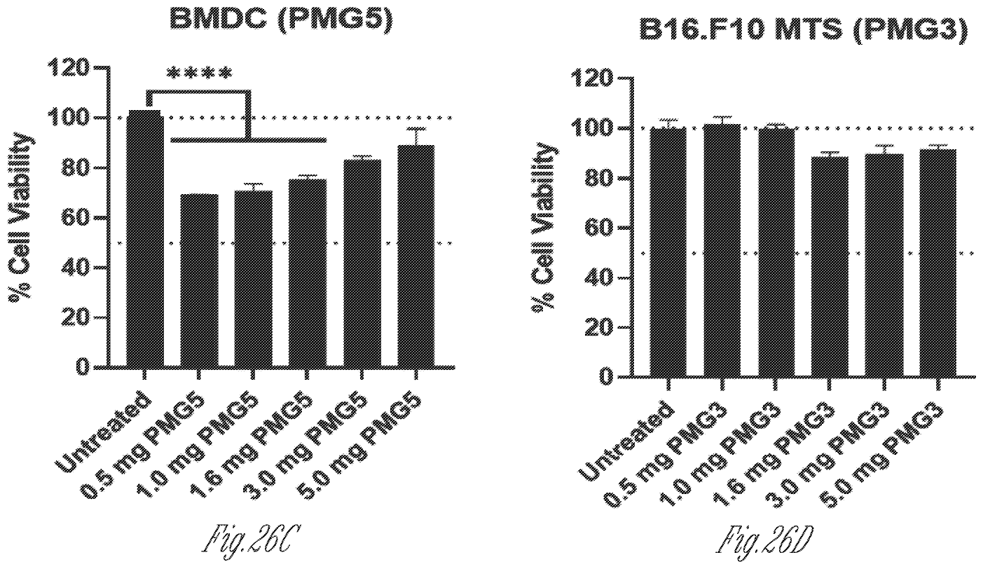

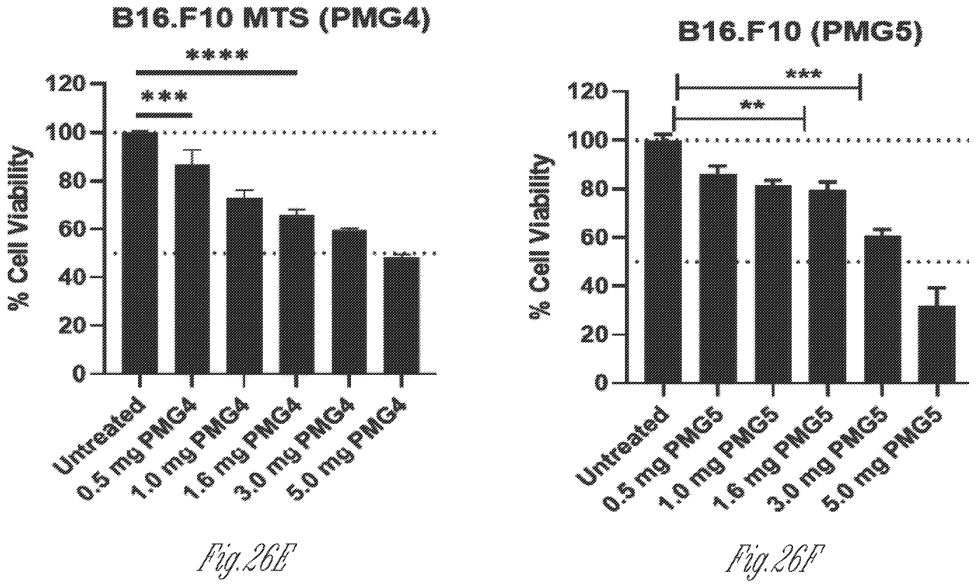

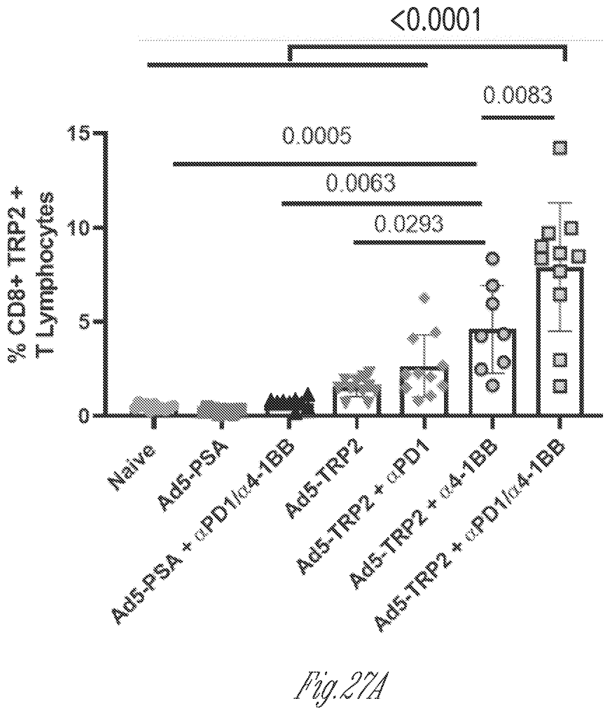

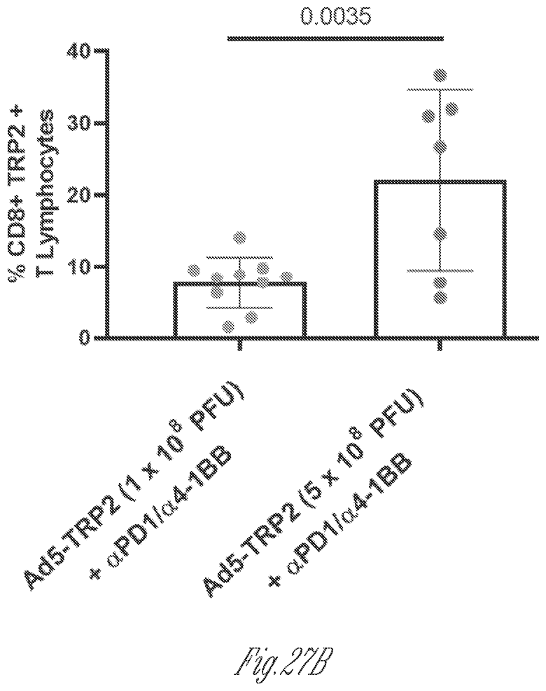

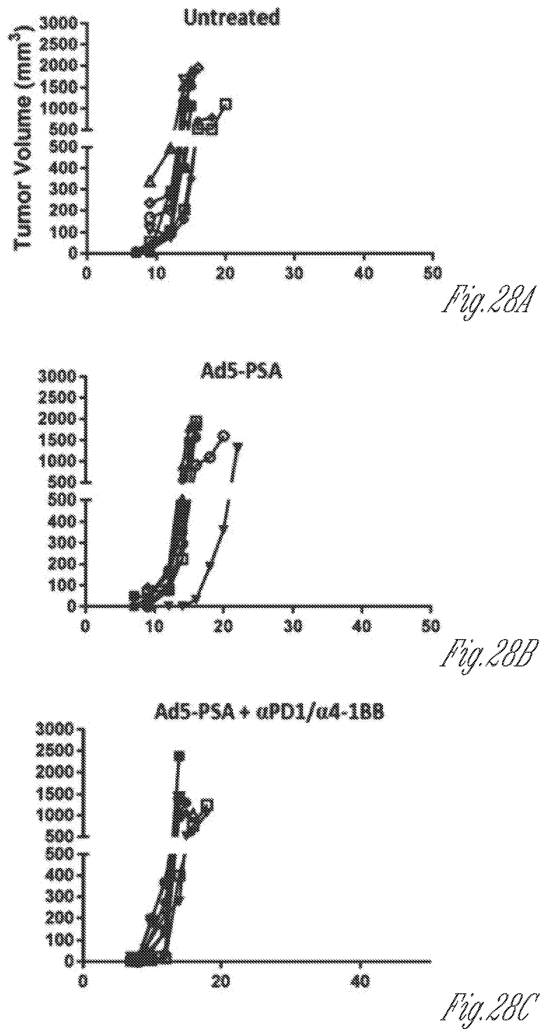

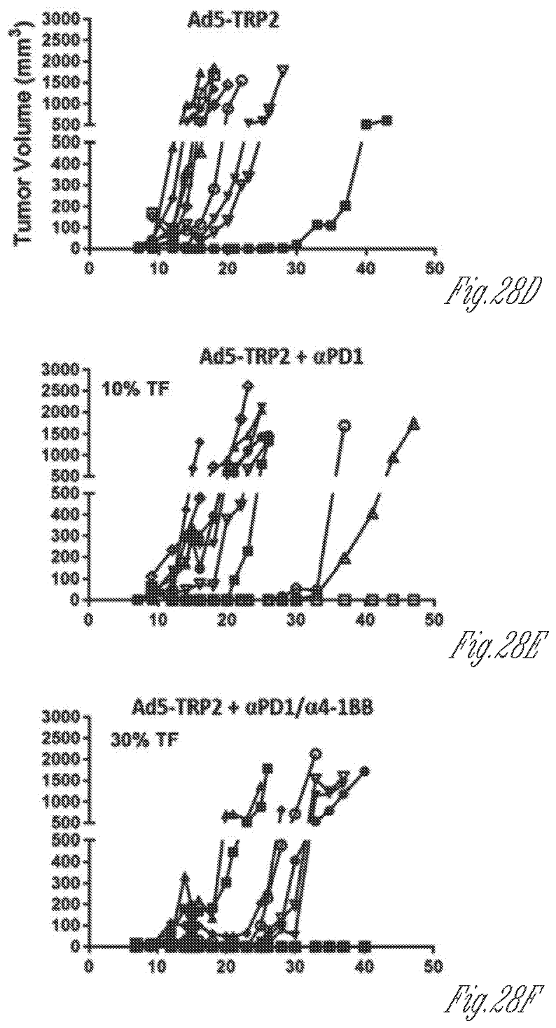

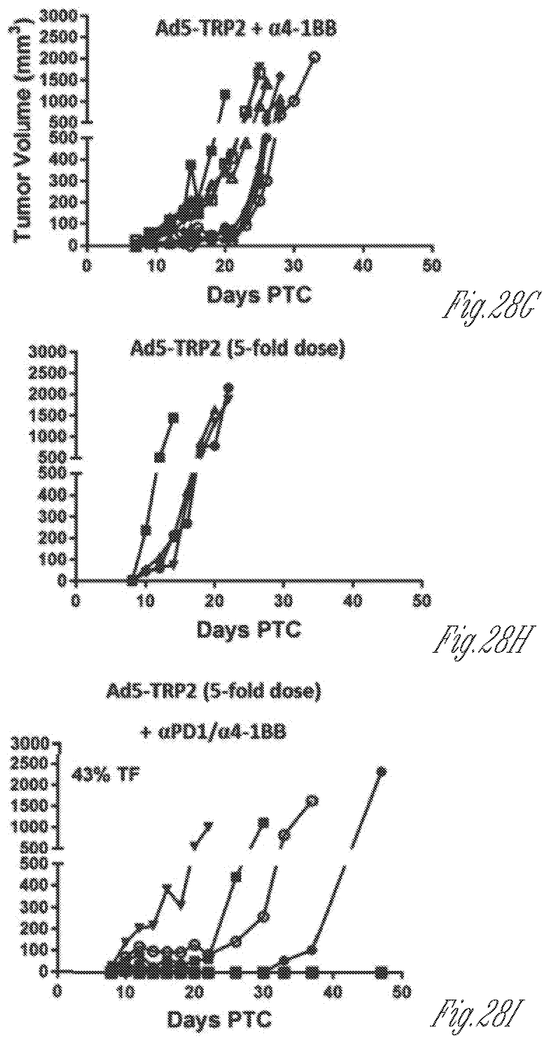

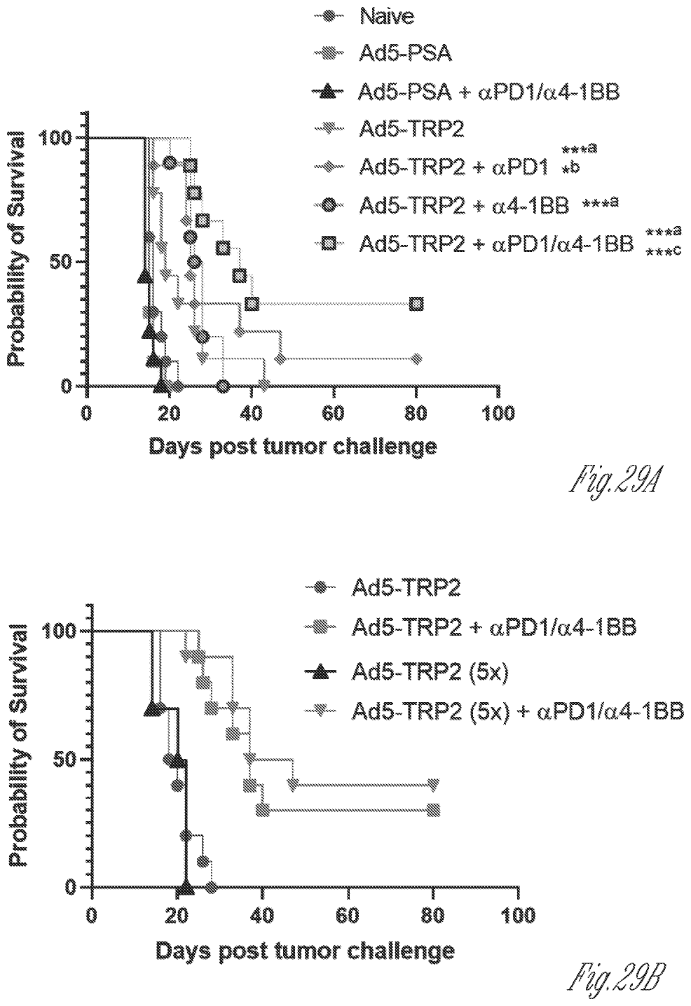

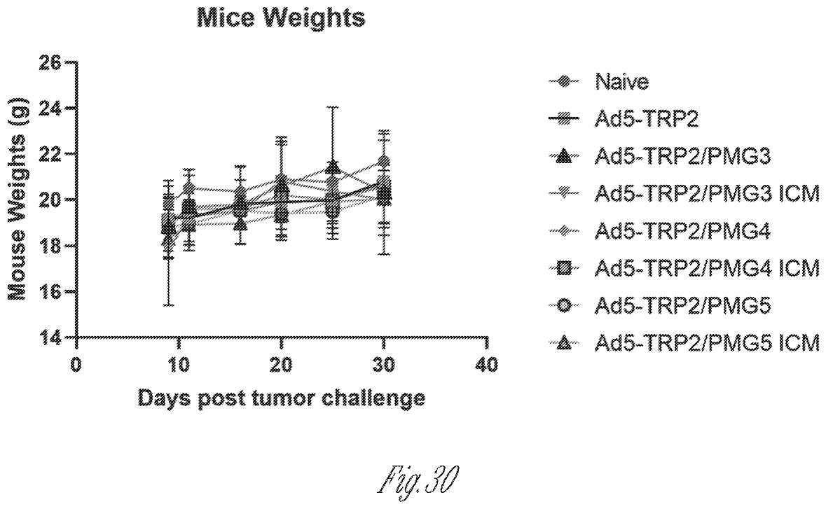

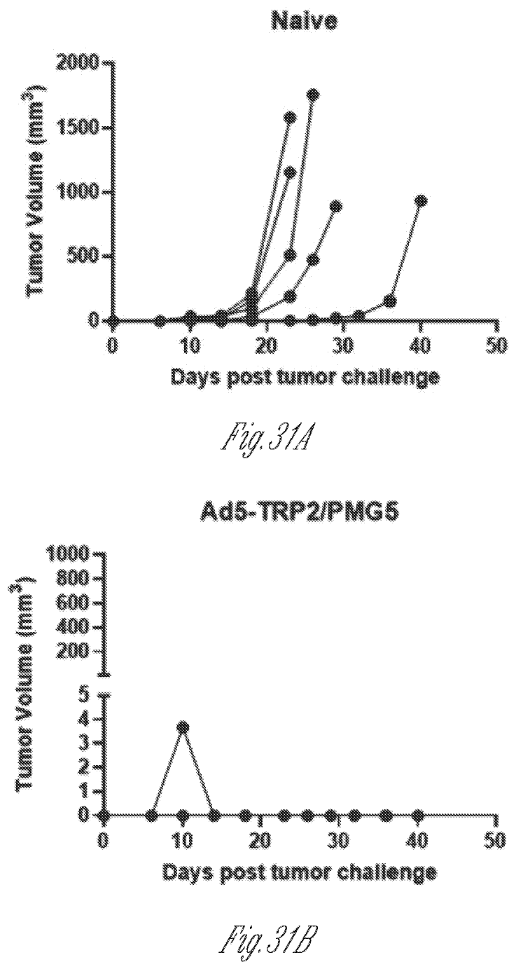

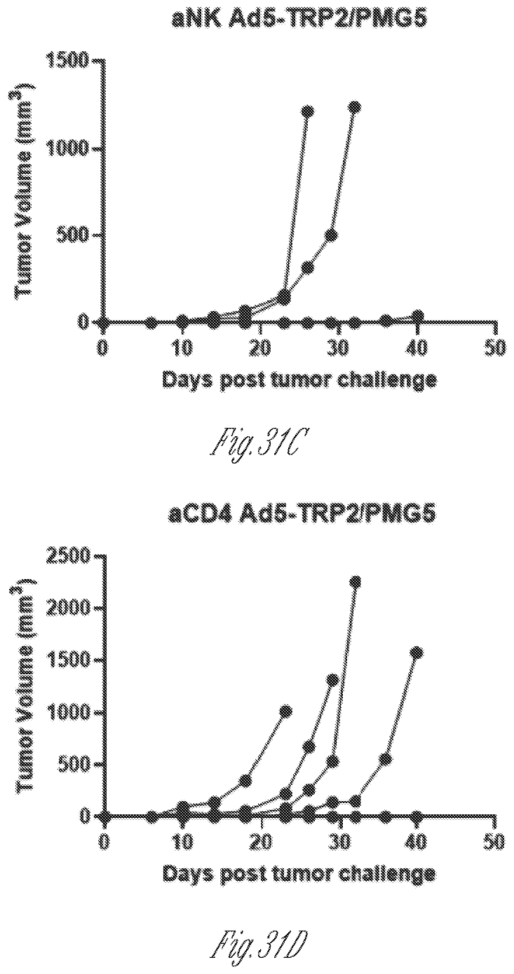

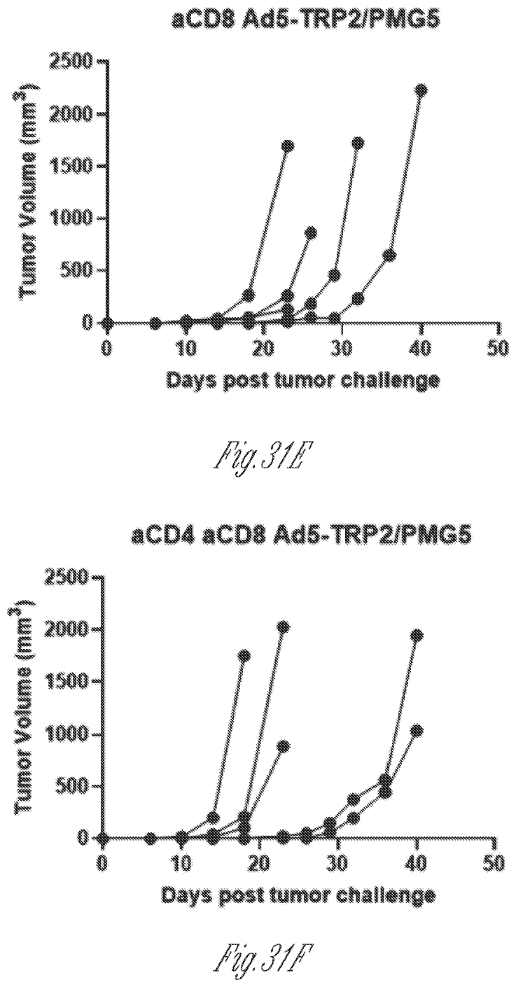

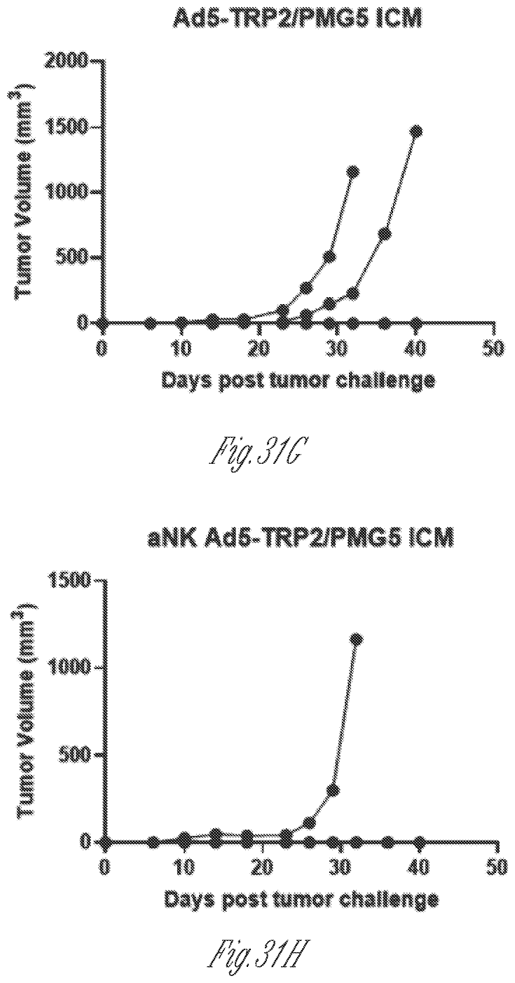

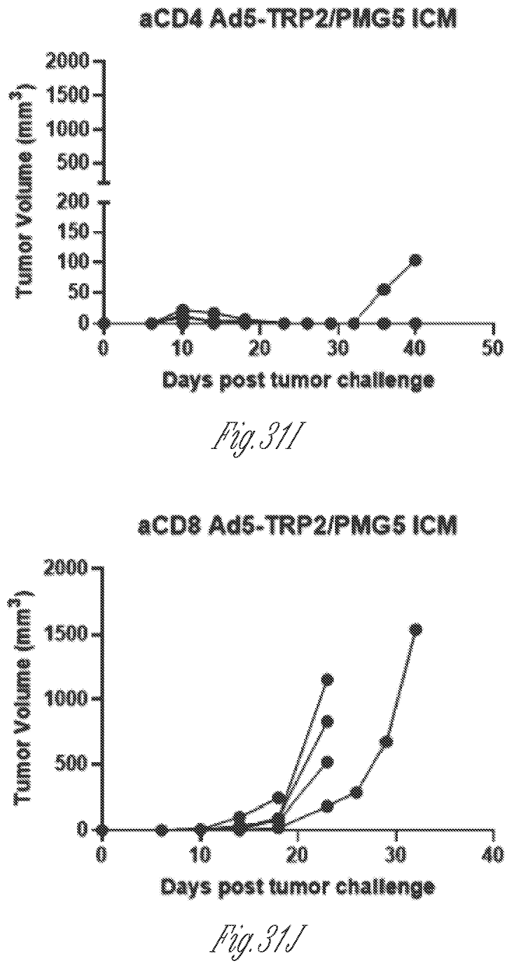

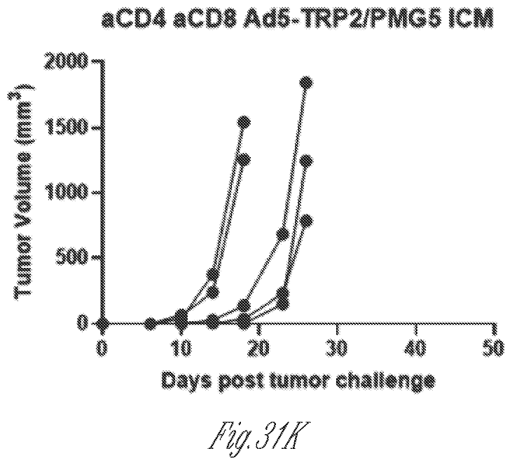

Given the antitumor efficacy observed when mice were treated with Ad5/PMG5, PMs were made using different generations of PAMAM (generation 3 and 4, ethylenediamine core). The diameter of the PM seems to increase with decreasing generation, e.g., PM made using generation 3 PAMAM (PMG3) had a diameter of 231 t 2.55 nm while PM made using generation 4 PAMAM (PMG4) had a diameter 170.83 t 1.95 nm (FIGS. 10A and D). The surface charges of PMG3 and PMG4 were (+39.03±1.67) mV and (+42.97±0.55) mV, respectively (FIGS. 9C and 9F).

The PMG4 and PMG3 also proved to be stable with small changes in size and surface charge after 30 days in nanopure water at 37° C. and room temperature (RT) (FIGS. 11A and 11D). The PDI of both PMG3 and PMG4 appeared to be stable at RT for 40 days. However, for PMG4, in particular, incubating beyond 10 days at 37° C., the PDI progressively increased (FIG. 11E). PMG3 was not toxic to B16F10 cells at any of the concentrations tested; however, it was 30 to 50% toxic to BMDCs at all concentrations of PMG3 tested (FIGS. 12A and 12C). PMG4 was toxic to both B16.F10 and BMDCs in a dose dependent fashion (FIGS. 12B and 12D).

In vivo, the antitumor efficacy observed with Ad5/PMG5 was greater than Ad5/PMG3 and Ad5/PMG4 (FIGS. 13A-H). The median survival of mice treated with Ad5/PMG3 or Ad5PMG4 was 30 days and 33 days post tumor challenge (PTC), respectively while that for Ad5/G5 was 103 days PTC. The survivals at 60 days for mice treated with Ad5/PMG5, Ad5/PMG4 and Ad5/PMG3 were 66.6%, 20% and 0%, respectively (FIG. 13J). The level of TRP-2-specific T cells also progressively decreased in mice treated with Ad5/PM combinations when the PM was made from PAMAM of lower generations of PAMAM: Ad5/PMG3 (1.147%±0.28) Ad5/PMG4 (1.554%±0.71) and Ad5/PMG5 (2.076%±0.73) (FIG. 13I).

Combining either Ad5/PMG3, Ad5/PMG4 or Ad5/PMG5 with checkpoint blockade, e.g., αPD1 and a41BB, (CPB) significantly increased the amount of CD8+ TRP2-specific T cells in the peripheral blood: Ad5/PMG3/CPB (6.543±4.87), Ad5/PMG4/CPB (6.651±4.32), Ad5/PMG5/CPB (7.696±5.91) (FIG. 13I). Interestingly, the survival at 60 days was in contrast to the relative survival observed when no CPB was used: Ad5/PMG3/CPB having the highest survival with 70% survival at day 60 and Ad5/PMG4/CPB had 60% survival and Ad5/PMG5/CPB had a 50% survival rate.

### Discussion

Cancer vaccines hold enormous potential for the treatment of intractable tumors such as late stage melanoma and aim to overcome the immunosuppressive nature of the TME and mount and effective tumor-specific immune response to eradicate cancer cells. To achieve this, one or more of the following may be needed: an antigen to differentiate the cancerous cells from healthy cells as part of the vaccine formulation; a delivery vehicle to effectively deliver the antigen to the immune system (i.e. DCs) and an adjuvant which ensures the enhanced activation of a tumor-specific immune response capable of eradicating cancer cells. The goal of this study was to use PMG5 as an adjuvant in combination with Ad5-TRP2 to create a cancer vaccine to treat melanoma. Melanoma represents an excellent model for demonstrating the efficacy of cancer vaccines. Despite a wide repertoire of TAA and demonstrated responsiveness to certain immunotherapies, late stage melanoma represents a major health concern having just a 9-19% 5-year survival rate, depending on where the primary tumor metastasizes to (Gershenwaki et al., 2008). While the Ad5 vector used possesses a tropism for DCs, an adjuvant is still needed to elicit a strong immune response against the self-antigen, TRP2. To this end, understanding the effects of how PMG5 interact with DCs is crucial.

The present findings indicate that the small nano-sized PMG5 are taken up by BMDCs much more efficiently than larger micro sized particles made from the same PLGA and PAMAM solution, which is in agreement with previous studies were small, positively charged NPs (˜50-100 nm) were shown to be taken up more efficiently than larger (˜1000 nm) particles (Blank et al., 2013; Foged et al., 2005; Seydoux et al., 2014). Pretreatment of BMDCs with MβCD and amiloride significantly inhibited the uptake of PMG5; suggesting that the formulation is most likely taken up via a caveola-mediated pathway and micropinocytosis. Cationic nanoparticles entering the cell through a caveolin-dependent mechanism can sometimes escape lysosomal degradation (Carver & Schnitzer, 2003).

Due to the highly positive surface charge (i.e., ZP) of PMG5, and evidence that PMG5 is taken up by BMDCs, it was hypothesized that PMG5 may be used as a delivery vehicle for negatively charged adjuvants such as CpG and/or nucleic acids. Despite establishing an astounding loading of CpG with PMG5 (31.25 μg/mg CpG per PMG5) which is significantly higher than that of PLGA nanoparticles encapsulating CpG (5 μg/mg) (data not shown), complexing CpG with PMG5 seems to reduce the activity of CpG in-vitro possibly due to the strong electrostatic interactions between PMG5 and CpG. T cells are the major effector cells for cancer immunity. While CD8+ T cells can directly lyse cancer cells, CD4+ T cells greatly influence CD8+ T cell efficacy and in some cases can diminish it. CD4+ T cells may be polarized into different subtypes Th1, Th2, Th17, and Tregs (Cook et al., 2012; Swain, 1995). Th1 cells produce pro-inflammatory cytokines which assist CTL in killing tumor cells and create a hostile environment for the tumor (Mohsen et al., 2020). Other Th subsets are not desired particularly Th2, which produce cytokines which stimulate a IgE responses and dampen Th1 responses, and Tregs which inhibit the function of CTLs. It was shown here that, while PMG5 on its own does not seem to stimulate BMDCs, in the presence of Ad5-TRP2 it causes a higher Th1/Th2 ratio (FIG. 8).

Despite modest activation of BMDCs in vitro, in vivo results demonstrated a clear advantage of using PMG5 as an adjuvant over the established TLR9 agonist CpG, or the combination of CpG with PMG5. This can be seen with the elevated levels of TRP2-specific CD8+ T cells in the peripheral blood; the significant increase median survival and the protection against B16.F10 re-challenge in mice treated with Ad5/PMG5 (FIGS. 101 and 10K). It is important to emphasize that this promising result was demonstrated in a therapeutic setting where treatment of solid tumor models such as B16.F10 by cancer vaccines has often demonstrated poor responses. Previous successful attempts with similar efficacies have involved multiple administrations of high viral titers (Perricone et al., 2000) or by using either a multipronged non-viral approach involving multiple administrations of chemically modified proteins, adjuvants and checkpoint blockade agents (Moynihan et al., 2016). A possible explanation for this increased efficacy witnessed in the present studies is that the local inflammation caused by the highly positive PMG5 injection resulted in the recruitment of antigen specific T cells to the tumor site. Local inflammation has been shown before to cause recruitment of TRP2-specific T cells to the site of inflammation (Steitz et al., 2005).

To further evaluate this, PMs were made with generation 3 and 4 PAMAM, characterized and the in-vivo anti-tumor efficacy evaluated. The relative sizes of PM particles were: PMG3>PMG4>PMG5. The in-vivo efficacy observed with Ad5/PMG5 was not observed with the other of the PM formulations tested despite them generating similar peripheral antigen specific T cell responses. Combining the groups, Ad5/PMG3, Ad5/PMG4, Ad5/PMG5 with immune checkpoint inhibitors, αPD1 and a 41BB increased the therapeutic efficacy of Ad5/PMG3 and Ad5/PMG4, with Ad5/PMG3 demonstrating best survival with 70% survival followed by Ad5/PMG4 (60% survival) then Ad5/PMG5 (50% survival). While Ad5/PMG5 on its own seems to provide substantial protection against tumor challenge with a 60% survival at day 60, combining it with checkpoint blockade does not seem to impact the overall survival of mice (50% survival). This effect is reversed with Ad5/PMG3 where the survival at day 60 was raised from 0% for Ad5/PMG3 to 70% for Ad5/PMG3 CPB.

### Conclusion

In summary, a cationic NP formulation using PLGA and PAMAM (PM) was successfully synthesized and characterized. It was demonstrated that the combination of a single administration of Ad5-TRP2 with subsequent peritumoral administrations with PMG5 was effective in treating cancer in a murine melanoma model, and so the combination can be employed as a cancer vaccine. This treatment regimen not only stops tumor growth but protects mice from subsequent tumor challenges a response not observed with treatment of CpG, an established adjuvant currently being used in multiple clinical trials. This is evident from an elevated TAA-specific CD8+ T cell response, significant increase in median survival and prolonged survival of mice receiving this treatment regimen. A modest increase in overall survival of mice treated with Ad5 PMG4 and increase in antigen specific CD8+ T cells in the peripheral blood. It was also shown that combining PM formulations that were not PMG5 with CPB resulted in a significant increase in overall survival of mice. This demonstrates that this formulation can be combined with other immunotherapy modalities, e.g., adoptive cell transfer using TILs, TCR modified T cells of CAR T cells, or checkpoint blockade therapy, to yield additional antitumor efficacies. Further, the nanoparticles may be employed with any immune therapy including a vaccine, e.g., a coronavirus vaccine such as a SARS-CoV-2 vaccine. Given the strength of the nanoparticles as an adjuvant, booster doses may not be needed for at least some vaccines.

### Example 2

Cancer immunotherapy represents an important treatment strategy for patients with inoperable cancers such as late-stage melanoma. A primary goal of cancer immunotherapy is to break immune tolerance in the immunosuppressive tumor microenvironment (TME) and mount an efficient immune effector response against tumor cells. Several FDA-approved cancer immunotherapy strategies exist for melanoma treatment including checkpoint blockade (ipilimumab (anti-CTLA-4), pembrolizumab (anti-PD1)), oncolytic viral therapy (Talimogene laherparepvec), and combinational checkpoint blockade (nivolumab (anti-PD-1)+ipilimumab). One approach which has shown great promise in preclinical therapeutic settings is the use of cancer vaccines where adjuvants in combination with tumor-specific antigens (TSAs) or tumor-associated antigens (TAAs) are administered to generate tumor-specific cytotoxic T cell responses. Currently, Sipuleucel-T® is the only FDA-approved therapeutic cancer vaccine; this involves the reinfusion of the host's blood cells (containing dendritic cells (DCs)) pretreated and activated with a recombinant fusion antigen (GMCSF-PAP). Despite its 4.1 month increase in median survival time and an overall reduction in risk of death compared to the placebo group, the high cost (=$100,000 per patient) prohibits its widespread adoption. Currently, a personalized dendritic cell-based melanoma cancer vaccine is in a phase lib clinical trial (NCT02301611) but runs the risk of following the path of Sipuleucel-T® by having a high cost associated with it; thus, making it inaccessible to the majority of patients not adequately insured.

A viable and cost-effective alternative to cell-based vaccines that can be delivered without the need for harvesting tissues from the patient includes using viral-based vaccines; these are an efficient means of delivering DNA encoding TAAs or TSAs and eliciting effective cytotoxic T lymphocyte (CTL) responses. While each viral vector has its advantages and disadvantages, the replication-deficient adenovirus has been proven to have many advantages, and its few disadvantages can be readily surmounted. The replication-deficient serotype 5 adenovirus (Ad5) has well-documented production techniques, can produce high viral titer stocks and can encode relatively large DNA inserts which can result in multiple whole TAA/TSA expression. Along with high-efficiency gene transduction, the Ad5 has been shown to have a tropism for DCs, the most potent professional antigen-presenting cell (APC) population. Given that DCs are the basis for all current cell-based cancer vaccines with FDA approval or those in clinical trials, the Ad5 cancer vaccine may be a viable and less expensive alternative. Ad5 has also been proven to be well-tolerated while being highly immunogenic in humans. An unfortunate downside to using Ad5 is the reduction in efficacy due to neutralizing antibodies resulting from prior exposure to the wild-type virus, however, this can be readily circumvented using a gelatin matrix such as Gelfoam® to deliver the Ad5-based vaccine.

Ideally, TSAs would be preferred over TAAs as the immunogen of choice for a cancer vaccine due to their inherently greater immunogenicity. However, TSAs lack the possibility of being prepared on a large standardized scale as they are often patient-specific. Also, TAA-based vaccines can be produced ahead of diagnosis since it is already known that a patient diagnosed with a certain type of cancer will likely express well-defined TAAs. For example, patients with melanoma will possess tumors expressing a suite of defined TAAs (including TRP-2). However, regardless of whether TAA-based or TSA-based cancer vaccines are implemented, they both must overcome the myriad of immunosuppressive properties of the tumor microenvironment (TME). Numerous strategies have been tested in preclinical studies in an attempt to improve the potency of TAA-based and model TSA-based cancer vaccines. It has been previously demonstrated that combining the Ad5 cancer vaccine (carrying a model TSA) with intratumoral administration of the adjuvant, cytosine guanine oligonucleotide (CpG ODN), significantly reduces tumor growth and increases survival in mice, along with increasing the proportion of antigen-specific CD8+ T cells in the TME and peripheral blood. Adjuvants formulated into nanoparticles (NPs) may be effective at modulating the immunosuppressive nature of the TME. Studies have demonstrated the increased efficacy of adjuvants when formulated into NPs, and NPs of less than 500 nm have been shown to effectively accumulate in DCs.

Thus, formulating an NP-based adjuvant system may provide a possible means to further enhance the immunogenicity of Ad5-based cancer vaccines and cancer vaccines in general. Nanoscopic compounds such as dendrimers have created new avenues toward the development of novel delivery systems. Since being introduced in 1984, polyamidoamine dendrimers (PAMAM) have gained the attention of many researchers as a tool for gene delivery and drug delivery. Despite their great potential and biomedical applications, PAMAM are known to be toxic; however, this can be overcome through chemical modifications that shield the highly cationic surface. This of course limits the very attributes which differentiated PAMAM to begin with. In an effort to yield the beneficial attributes of PAMAM without reducing their biomedical application through chemical modification, a PAMAM-based NP formulation was developed utilizing a combination of poly (D,L-lactic-co-glycolic acid) (PLGA) and PAMAM to form NPs that limit the toxicities associated with unmodified PAMAM. This NP differs from previously reported formulations where PAMAM was physically adsorbed to the surface of PLGA NPs or the PAMAM was chemically modified to yield beneficial attributes. This PLGA/PAMAM NP formulation is termed PM and represents the first time that PLGA and PAMAM were used to make bilayer NPs from a single polymer solution. To investigate the potential adjuvant properties of PM, they were used in combination with an antitumor vaccine, Ad5 encoding the melanoma TAA, tyrosinase-related protein 2 (Ad5-TRP2). Melanoma represents an appropriate model to explore the effectiveness of this formulation for several reasons. The NIH Surveillance and Epidemiology and End Results Program (SEER) places malignant melanoma as the fifth most commonly diagnosed cancer in the United States with an estimated 96,480 new cases in 2019. Melanoma, if detected early (melanoma in-situ), can be treated with surgery or targeted therapeutic agents based on a patient's mutation status; however, for advanced-stage melanoma (stage Ill or IV) few options are available to patients. Thus, immunotherapy represents a viable alternative for these patients. Here, it was demonstrated that therapeutically combining PM with Ad5-TRP2 resulted in inhibition of tumor growth and increased survival in melanoma-challenged mice, which was in part due to greatly increased systemic CD8+ T cell responses. Mice that survived tumor-free long-term after receiving this treatment were also protected from tumor re-challenge.

### Materials and Methods

### Animals

All animal experiments involved 6-8-week-old, female C57/B6J mice that were purchased from Jackson Laboratories and housed in the University of Iowa animal care facility for a minimum of 1 week before use. All mice were maintained in filtered cages. All animal experiments were carried out in accordance with guidelines and regulations approved by the University of Iowa Institutional Animal Care and Use Committee.

### Adenovirus Synthesis

The attenuated serotype 5 adenoviruses (Ad5) used in these studies were manufactured by the Viral Vector Core (The University of Iowa, Carver College of Medicine, Iowa City, IA) using a method described in Anderson et al. (2000). The viral DNA construct was engineered to express human tyrosinase-like protein-2 (TRP2) upon transduction of living cells. All Ad5 had a portion of the adenoviral genome deleted that included the left-hand terminal repeat, the packaging signal, and E1A and E1B sequences, rendering the virus replication deficient.

### PLGA/PAMAM NP (PMG5, PMG4) Formulations

PLGA PAMAM nanoparticles (PM) were fabricated using a modified nanoprecipitation method. Briefly, 50 mg of 50:50 poly(D,L-lactide-co-glycolide) (PLGA, Resomer® RG 502) was dissolved in 5 mL acetone, then, 125 μL of polyamidoamine (PAMAM) dendrimer (ethylenediamine core, generation 5 or generation 4, Sigma Aldrich, St. Louis, MO) was added to make a resulting PAMAM solution at a concentration of 0.125% w/v. PLGA/PAMAM solution was added dropwise from a syringe needle (G26) (without applied pressure; i.e. gravity dependent) into a 0.1% w/v PVA aqueous solution (MW 9000-10000 g/mol, Sigma Aldrich) under stirring. Following this, instantaneously formed PMs were stirred in a fume hood for 30 minutes. PMs were then transferred to a rotary evaporator (Buchi, Rotavapor R-300, Switzerland), which was set to 50 mBar at 50 rpm for 4 hours to evaporate organic solvents. Then PMs were washed with sterilized DI water in Amicon™ Ultra, centrifuge tubes (100,000 MWCO) 4 times by centrifuging at 500×g for 1 hour. PMs were then frozen in a 10% sucrose solution at −80° C. for 24 hours, then lyophilized for 48 hours using a freeze dryer (Labconco, Freezone—4.5 L, Kansas City, MO). Finally, dry PMs were collected and stored in sealed containers until use. To evaluate the impact of involving the lyophilization process and adding sucrose to the formulation, two other different formulations of PMs were prepared; one was freshly prepared (i.e., no freezing or lyophilization) while the other batch was prepared and lyophilized in the absence of cryoprotectant (i.e., containing no sucrose). PMs synthesized using PAMAM dendrimer (ethylenediamine core, generation 5) were designated as PMG5; and those synthesized using PAMAM dendrimer (ethylenediamine core, generation 4) were designated PMG4.

### PLGA/PAMAM G3 NP (PMG3) Formulation

Due to its high methanol content (20 wt. %), 125 μL PAMAM dendrimer (ethylenediamine core, generation 3, Sigma Aldrich)) was placed in a rotary evaporator (Buchi, Rotavapor R-300, Switzerland), which was set to 50 mBar at 50 rpm for 30 minutes to evaporate organic solvents. PAMAM was then resuspended in 125 μL of DMSO. This solution was then used to synthesize PMs using the fabrication method listed above with generation 4 and 5 PAMAM. PMs synthesized using PAMAM dendrimer (ethylenediamine core, generation 3) were designated as PMG3.

### Characterization of PM Formulations

PMG5 were imaged using transmission electron microscopy (TEM) (JEOL, JEM-1230, Tokyo, Japan). Briefly, 0.1 mg/mL of lyophilized PMG5 was added to 1.5% (v/v) sterile filtered phosphotungstic acid at a ratio of 1:1. The resulting solution was placed on carbon-coated TEM grids and imaged to analyze the morphology of PMG5. PMG5 were further characterized by assessing the average hydrodynamic diameter, polydispersity index (PDI), and net surface charge using a Zetasizer Nano ZS (Malvern Panalytical, Malvern, United Kingdom) as previously described (65-68). In addition, the shape and surface morphology of PMG5 were further examined using a Hitachi scanning electron microscope (SEM) (Hitachi High-Technologies, Schaumburg, Illinois) (65-68). PLGA, PAMAM, and PMG5 were also characterized for heat flow properties by analyzing the thermograms obtained from the differential scanning calorimeter (DSC Q20, TA Instruments, New Castle, DE) equipped with a refrigerated cooling system (RCS90) (TA Instruments, New Castle, DE), as previously described (68). All samples were sealed in standard aluminum sample pans covered with lids. An empty sealed aluminum pan covered with a lid was used as a reference. Pure dry nitrogen (set at 20 psi pressure and 40 mL/min flow rate) was used as a purge gas. Samples were heated from 0° C. to 100° C. at a 5° C./min heating rate.

### Degradation Studies

In this experiment, 5 mg of lyophilized PMG3, PMG4, and PMG5 were dissolved in 5 mL of Nanopure water. The resulting solutions were placed at room temperature or 37° C. and agitated at 300 rpm for 42 days. Samples were taken at various time points (every day for 3 days, then every week for two weeks, then every two weeks) and the size, zeta-potential, and PDI were recorded using the Zetasizer Nano ZS.

### Evaluation of the Effect of Lyophilization

Due to the intrinsic fluorescence of PAMAM, the cellular uptake behavior of PMG5 could be directly analyzed by fluorescence microscopy and flow cytometry, without additional fluorescence labeling (Tsai et al., 2011). Herein, quantitative and qualitative cellular uptake of PMG5 by bone marrow derived dendritic cells (BMDCs) was studied. For tracking the in vitro cellular uptake of PMG5, BMDCs were incubated with 0.12 mg of differently prepared PMG5 as follows: (i) non-lyophilized PMG5 (freshly prepared), (ii) lyophilized PMG5 in the presence of sucrose, (iii) lyophilized PMG5 without sucrose, and untreated BMDCs as a control (iv). Cells were then collected (without using trypsin; instead, vigorous flushing was implemented) and centrifuged (230×g) for 5 min at 4° C. All cell samples were run through a BD FACScan flow cytometer (Becton, Dickinson, Franklin Lakes, NJ) in triplicate, and data were analyzed with FlowJo software (Tree Star, Ashland, OR).

### PM Uptake by BMDCs

BMDCs were incubated with four different doses (0.06, 0.12, 0.18, and 0.24 mg) of lyophilized PMG5 with sucrose to assess the effect of the dose on the uptake capacity. After incubation for 48 hours, cells were collected, and samples were acquired using a FACScan flow cytometer. BMDCs were initially seeded in a 12-well plate in media-containing serum, as described above. Prior to adding the PMG5, the media was removed, and fresh serum-free media was added. This was followed by adding the PMG5 (lyophilized with sucrose) to the BMDCs. Cells were then collected and analyzed using a FACScan flow cytometer and FloJo software. A similar procedure was followed for PMG3 and PMG4 except that only one dose of the PM formulation (0.24 mg) was used.

### Mechanistic Study of the Uptake of PMG5 by BMDCs

This experiment was performed to study the mechanism(s) of uptake of PMG5 by BMDCs using inhibitors of endocytosis pathways. In general, endocytosis is an energy-dependent process that can be delineated as clathrin-mediated endocytosis, caveolae-mediated endocytosis, and macropinocytosis. In this study, BMDCs were incubated with different endocytosis pathway inhibitors including (i) sucrose to inhibit clathrin-mediated endocytosis (400 μM, added to the cells 1 hour prior to treating with PMG5), (ii) methyl-β-cyclodextrin to inhibit caveolae-dependent endocytosis (1 mM, added to the cells 1 hour prior to treating with PMG5), and (iii) amiloride to inhibit macropinocytosis (2 mM, added to the cells 10 minutes prior to treating with PMG5). This was followed by adding the PMG5 (0.12 mg) and incubating it with cells for 3 hours. BMDCs were then harvested and then data acquired and analyzed using a FACScan flow cytometer and FloJo software.

### Harvesting BMDCs and BMDC Activation Studies

BMDCs were generating following a procedure previously published. In brief, femurs and tibia were harvested from C57BL/6J mice and then the bone marrow was flushed with Roswell Park Memorial Institute (RPMI) 1640 cell culture media supplemented with 0.01 M HEPES buffer (Gibco, Thermo Fischer Scientific, Waltham, MA), 1 mM sodium pyruvate (Gibco, Thermo Fischer Scientific), 1×glutamax (Gibco, Thermo Fischer Scientific), 50 mM 2-mercaptoethanol (Sigma Aldrich), 0.5 mg/mL gentamycin sulfate (IBI Scientific, Dubuque, IA), and 10% fetal bovine serum (Atlanta Biologicals, Flowery Branch, GA). Bone marrow cells were then counted and seeded at a density of 2×10⁶ cells in 10 mL complete RPMI medium with 20 ng/mL GM-CSF (granulocyte-monocyte colony-stimulating factor) (Peprotech, Rocky Hill, NJ) at 37° C. with 5% CO₂ in bacteriological Petri dishes. On day 3 (72 hours after seeding) 10 mL of RPMI complete media with 20 ng/mL of GM-CSF was added. On days 6 and 8, 10 mL of cell culture supernatant was harvested and spun at 230×g, old media (supernatant) was aspirated and 10 mL of fresh media with 20 ng/mL GM-CSF was added. On day 10, cells were transferred to 12-well tissue culture grade plates and seeded at a density of 1×10⁵ cell/well and allowed to equilibrate for at last 6 hours before incubating with experimental groups (as described in the above section) then for 2 days within 2 mL complete media and experimental groups.

Supernatants from the cells were then collected and stored for later use in Luminex multiplex cytokine assays, and BMDCs were then flushed from the plate with ice-cold 1×DPBS (Gibco, Thermo Fischer Scientific). BMDCs were collected, washed with ice-cold FACS buffer (1×DPBS, 0.1% sodium azide, and 5% FCS), and transferred to a 96 well U-bottom plate (Cell star, Greiner). Cells were then incubated with a 1/100 dilution of anti-mouse CD16/CD32 (clone 93, Invitrogen) for 15 minutes on ice. BMDCs were then incubated with one or more of the following antibodies: anti-mouse CD11c FITC (clone N418, Invitrogen), anti-mouse CD40 APC (clone 3/23, Biolegend), anti-mouse CD80 APC (16-10A1), anti-mouse MHC class I (H-2Kb) PE (AF6-88.5.5.3, Invitrogen) for 30 minutes on ice in the dark. BMDCs were then washed twice with ice-cold FACS buffer and resuspended in 100 μL of Cytofix buffer (BD Biosciences) and allowed to incubate for 10 minutes on ice in the dark. Next, 100 μL of ice-cold 1×Perm/Wash solution (BD Biosciences) was added to each well, then cells were centrifuged at 660×g and resuspended in ice-cold FACS buffer and stored sealed at 4° C. until they were acquired using the FACScan flow cytometer and analyzed FlowJo software.

### Prophylactic B16.F10 Model

C57BL/6J female mice (n=5 per treatment group) were first vaccinated subcutaneously (SC) in the right-hand side rear dorsal flank with the indicated dose of Ad5-TRP2. On day 14 post-vaccination, mice were submandibular bled, and the harvested peripheral blood lymphocytes (PBLs) were stained for TRP-2 specific CD8+ T cells using the method described below. Also, on day 14 post-vaccination, the mice were challenged SC with 1×10⁵ live B16.F10 cells (in 100 μl of serum-free DMEM) on the left-hand side rear dorsal flank (contralateral to the vaccination). Mice were monitored for tumor size using calipers and tumor volume was determined based on the assumption that the tumors were ellipsoid in shape: [V=(Diameter 1×Diameter 2×Height)×(π/6)]. Mice were humanely sacrificed upon reaching end-point criteria which included possessing a tumor that had reached 20 mm in length or width or 10 mm in height. The experiment was terminated on day 55 post tumor challenge (PTC).

### Tumor Challenge and Treatment Protocol: Therapeutic B16.F10 Model

C57/B16J mice were randomly divided into 12 groups at 9-10 mice per group, as described in Table 1. Mice were then challenged on the dorsal right flank with 2×10⁵ live B16.F10 cells in 100 μL serum-free DMEM complete media. The following day, mice designated to receive Ad5-TRP2 were given a single dose contralaterally at 1×10⁸ PFUs. On days 8, 11, and 13 PTC, mice were given their designated treatment groups under anesthesia at the site of tumor inoculation on the right dorsal flank of the mouse. Tumor volumes and mice weights were recorded every 2-3 days. At 60 days post-initial tumor challenge, mice that did not develop tumors initially were re-challenged with 2×10⁵ cells on the dorsal right flank. On days 8, 11, 13, 16 and 18 PTC, select groups were administered with αPD1 (100 μg/mouse/administration) and/or α4-1BB (intraperitoneally (IP) at 100 μg/mouse/administration) in order to provide immune checkpoint modulation (ICM). Endpoint criteria were met when tumors reached 20 mm in length or width or 10 mm in height. Tumors were assumed to be ellipsoid in shape and volumes were recorded by measuring the length width and height with calipers and calculated using the formula:

$$Volume = ( length \times width \times height ) \times \frac{π}{6}$$

Throughout the study, mice were monitored for general signs of distress/toxicity including a reduction in activity/responsiveness to touch, hunched back, glassy coating over the eyes, significant (≥20%) weight reduction.

### Ex Vivo Staining of TRP2-Specific CD8+T Lymphocytes

At 15 days PTC, 180 μL of blood was collected by submandibular bleeding and mixed with ACK buffer (150 mM NH₄Cl, 10 mM KHCO₃, 0.1 mM Na₂EDTA) to lyse red blood cells. After a 10-minute incubation at room temperature, cells were washed (centrifuged at 230×g twice) using complete growth media. The cells were resuspended at <10⁷/mL (in ice-cold PBS containing 2% v/v FCS and 0.05% w/v sodium azide: FACS buffer) and transferred to 98-well V-bottomed trays. Cells were then centrifuged at 4° C. at 230×g for 5 minutes, then the supernatant was removed. Cell pellets were then resuspended in 50 μL anti-mouse CD16/CD32 and incubated on ice for 15 minutes. Then, 50 μL of tetramer stain diluted 1/100 in FACS buffer was added and cells were incubated on ice in the dark for 30 minutes. Next, 100 μL of antibody cocktail [anti-CD8a-FITC (1/400, clone 53-6.7, ebiosciences), anti-CD3-PECy5 (1/200, clone 145-2C11, ebiosciences)] was added to the cells, and cells were then incubated for 20 minutes on ice in the dark. Cells were then washed twice with FACS buffer and resuspended in 100 μL Cytofix solution and incubated on ice in the dark for 10 minutes. Finally, 100 μL of 1×Perm/Wash solution (BD Biosciences) was added to each well, then cells were spun at 660×g, resuspended in FACS buffer, and stored sealed at 4° C. until they were acquired using the FACScan flow cytometer and analyzed FlowJo software

### Depletion Studies

Mice were randomly divided into 10 groups (5 mice per group), as described in Table 2. Mice were then challenged on the dorsal right flank with 2×10⁵ live B16.F10 cells in 100 μL serum-free DMEM complete media. The following day, mice designated to receive Ad5-TRP2 were given a single dose contralaterally at 1×10⁸ PFUs. On days 8, 11, and 13 PTC, mice were given their designated PM treatments under anesthesia at the site of tumor inoculation on the right dorsal flank of the mouse. Tumor volumes and mice weights were recorded every 2-3 days. On days 8, 11, 13, 16 and 18 PTC, select groups were administered with αPD1 (100 μg/mouse/administration) and α4-1BB (IP at 100 μg/mouse/administration). On days 6, 7, 8 PTC then every 3 days until day 30 PTC designated groups received 150 μg of antibodies (αNK, clone number PK136; αCD4, clone number GK 1.5; αCD8, clone number 2.43) IP to deplete specified immune cell populations. Endpoint criteria were met when tumors reached 20 mm in length or width or 10 mm in height. Tumors were assumed to be ellipsoid in shape and volumes were recorded by measuring the length, width and height with calipers and calculated using the formula:

$$Volume = ( length \times width \times height ) \times \frac{π}{6}$$

### Statistical Analysis

Unless indicated otherwise statistical differences were analyzed by one-way ANOVA analysis of variance followed by a Turkey post-test to compare all pairs of treatment. Where indicated statistical differences were analyzed by two-way ANOVA analysis of variance followed by a Turkey post-test. Survival curves were analyzed using adjustments for multiple comparisons for the log-rank (Mantel-Cox) test using Dunnett's method. All statistical tests were performed using GraphPad Prism software (Prism 5, Version 9.0.0, La Jolla, CA).

TABLE 3
List of the experimental groups used in animal
studies and in vitro BMDC activation studies.

| Group Name | Group Description | Dose (per mouse in 100 μL) |
|---|---|---|
| Naïve | Untreated Mice | — |
| Ad5-TRP2 | Adenovirus Serotype 5 | 1 × 10⁸ PFUs |
| | encoding tyrosinase- | |
| | related protein-2 | |
| PMG5 | PMG5 only | 1.6 mg |
| CpG | Soluble CpG B | 50 μg |
| Ad5-TRP2/ | Ad5-TRP2 with PMG5 | 1 × 10⁸ PFUs with 1.6 mg |
| PMG5 | | of PMG5 |
| Ad5-TRP2/ | Ad5-TRP2 with Soluble | 1 × 10⁸ PFUs with 50 μg |
| CpG | CpG B | of CpG |
| Ad5-TRP2/ | Ad5-TRP2 with PMG3. | 1 × 10⁸ PFUs with 1.6 mg |
| PMG3 | | of PMG3 |
| Ad5-TRP2/ | Ad5-TRP2 with PMG4. | 1 × 10⁸ PFUs with 1.6 mg |
| PMG4 | | of PMG4 |
| Ad5-TRP2/ | Ad5-TRP2 with PMG5. | 1 × 10⁸ PFUs with 1.6 mg |
| PMG5 | | of PMG5. |
| Ad5-TRP2/ | Ad5-TRP2 with PMG3 | 1 × 10⁸ PFUs with 1.6 mg |
| PMG3 ICM | and αPD-1 and α4-1BB. | of PMG3 with 100 μg of |
| | | αPD-1 and 100 μg of α4-1BB. |
| Ad5-TRP2/ | Ad5-TRP2 with PMG4 | 1 × 10⁸ PFUs with 1.6 mg |
| PMG4 ICM | and αPD-1 and α4-1BB. | of PMG4 with 100 μg of |
| | | αPD-1 and 100 μg of α4-1BB. |
| Ad5-TRP2/ | Ad5-TRP2 with PMG5 | 1 × 10⁸ PFUs with 1.6 mg |
| PMG5 ICM | and αPD-1 and α4-1BB. | of PMG5 with 100 μg of |
| | | αPD-1 and 100 μg of α4-1BB. |

TABLE 4
List of the experimental groups used in animal
depletion studies. α = anti

| Group Name | Group Description | Dose (per mouse in 100 μL) |
|---|---|---|
| Ad5-TRP2/ | Ad5-TRP2 with | 1 × 10⁸ PFUs with 1.6 mg |
| PMG5 | PMG5. | of PMG5. |
| Ad5-TRP2/ | Ad5-TRP2 with | 1 × 10⁸ PFUs with 1.6 mg |
| PMG5 ICM | PMG5 and αPD-1 | of PMG5 with 100 μg of |
| | and α4-1BB. | αPD-1 and 100 μg of |
| | | α4-1BB. |
| αNK Ad5- | Ad5-TRP2 with | 1 × 10⁸ PFUs with 1.6 mg |
| TRP2/PMG5 | PMG5 and αNK | of PMG5 with 150 μg of |
| | antibodies. | αNK. |
| αCD4 Ad5- | Ad5-TRP2 with | 1 × 10⁸ PFUs with 1.6 mg |
| TRP2/PMG5 | PMG5 and αCD4 | of PMG5 with 150 μg of |
| | antibodies. | αCD4. |
| αCD8 Ad5- | Ad5-TRP2 with | 1 × 10⁸ PFUs with 1.6 mg |
| TRP2/PMG5 | PMG5 and αCD8 | of PMG5 with 150 μg of |
| | antibodies. | αCD8. |
| αCD4 αCD8 | Ad5-TRP2 with | 1 × 10⁸ PFUs with 1.6 mg |
| Ad5-TRP2/PMG5 | PMG5 and αCD8 and | of PMG5 with 150 μg of |
| | αCD4 antibodies. | αCD8 and αCD4. |
| αNK Ad5- | Ad5-TRP2 with | 1 × 10⁸ PFUs with 1.6 mg |
| TRP2/PMG5 ICM | PMG5 and αPD-1 | of PMG5 with 100 μg of |
| | and α4-1BB and | αPD-1 and 100 μg of α4- |
| | αNK antibodies. | 1BB and 150 μg of αNK. |
| αCD4 Ad5- | Ad5-TRP2 with | 1 × 10⁸ PFUs with 1.6 mg |
| TRP2/PMG5 ICM | PMG5 and αPD-1, | of PMG5 with 100 μg of |
| | α4-1BB and αCD4 | αPD-1 and 100 μg of α4- |
| | antibodies. | 1BB and 150 μg of αCD4. |
| αCD8 Ad5- | Ad5-TRP2 with | 1 × 10⁸ PFUs with 1.6 mg |
| TRP2/PMG5 ICM | PMG5 and αPD-1, | of PMG5 with 100 μg of |
| | α4-1BB and αCD8 | αPD-1 and 100 μg of α4- |
| | antibodies. | 1BB and 150 μg of αCD8. |
| αCD4 αCD8 | Ad5-TRP2 with | 1 × 10⁸ PFUs with 1.6 mg |
| Ad5-TRP2/PMG5 | PMG5 and αPD-1, | of PMG5 with 100 μg of |
| ICM | α4-1BB and αCD8 | αPD-1 and 100 μg of α4- |
| | antibodies. | 1BB and 150 μg of αCD8 |
| | | and αCD4. |

### Results

### Particle Synthesis, Characterization, and Uptake

PAMAM can be classified according to a generation number, referring to the degree of branching from the core and being related to the number of surface amines present on the PAMAM. Initially, PM were synthesized using the nanoprecipitation method summarized in (FIG. 14) from a mixture of PLGA and PAMAM (ethylenediamine core, Generation 5) and the resultant NPs will be referred to as PMG5. Transmission electron microscopy (TEM) and scanning electron microscopy (SEM) images showed that PMG5 were spherical with smooth surfaces (FIG. 14B-D). Fabricating PMG5 prior to each use is not practical for translation into the clinic. Thus, to evaluate their stability under appropriate storage conditions, PMG5 were lyophilized (freeze-dried) with and without sucrose, a cryoprotectant. The lyophilization of PMG5 without sucrose resulted in significant aggregation as indicated by an increase in the size and polydispersity index (PDI) of the PMG5 (FIGS. 14E and 14F). This effect was circumvented by adding sucrose to the formulation which resulted in a significant decrease in the size and PDI of PMG5 when compared to PMG5 lyophilized without sucrose. It has been previously established that PAMAM exhibit intrinsic fluorescence in the blue region with a slight shift in lambda max with increasing generations (Konopka et al., 2018; Waade et al., 2001; Caminati et al., 1990). Taking advantage of this feature, the intrinsic fluorescence of PAMAM was used to evaluate the uptake of PMG5 by bone marrow-derived dendritic cells (BMDCs) and fluorescence (an indicator of uptake) was assessed by flow cytometry. Results shown in FIG. 15D demonstrate clearly that BMDCs incubated with PMG5 (163.9 t 0.61 nm) had significantly greater fluorescence (due to the PMG5 being taken up by BMDCs) than BMDCs incubated with larger submicron (523.9 t 15 nm) and micron-sized particles (1278.3±27 nm) (also made from PLGA and PAMAM) after 48 hours incubation.

The effect of using sucrose as a stabilizer during lyophilization on PMG5 uptake by BMDCs was further evaluated by incubating BMDCs with freshly prepared PMG5 and PMG5 lyophilized with and without sucrose. It was observed that PMG5 lyophilized with sucrose were taken up more efficiently by BMDCs compared to PMG5 lyophilized without sucrose (FIG. 15E). Thus, it would appear that because sucrose preserved the size and PDI of lyophilized PMG5 (and therefore were similar to freshly prepared PMG5 as shown in FIG. 14), their capacity to be taken up by BMDCs was also mostly preserved (FIG. 15E). Thus, PMG5 lyophilized with sucrose were used in subsequent studies. Treatment of BMDCs with different doses of PMG5 revealed a dose-dependent rate of uptake (FIG. 2F). The uptake mechanism of PMG5 was investigated by incubating PMG5 with BMDCs pretreated with inhibitors of three distinct pathways of endocytosis: amiloride (an inhibitor of macropinocytosis), methyl-β-cyclodextrin (MβCD) (an inhibitor of clathrin-independent endocytosis), and a high concentration (400 μM) of sucrose (an inhibitor of clathrin-mediated endocytosis)). As expected, incubation of BMDCs (not treated with any inhibitor) with PMG5 displayed a significant increase in the mean fluorescence intensity (MFI) compared to untreated BMDCs (cells not incubated with PMG5) (FIG. 15G). Incubation of PMG5 with BMDCs pretreated with a high concentration of sucrose did not result in a significant reduction in the MFI when compared to BMDCs incubated with PMG5 in the absence of an inhibitor. Interestingly, PMG5 incubated with BMDCs pretreated with either amiloride or MPCD exhibited a significant reduction in the MFI in comparison to PMG5 incubated with BMDCs (without inhibitors). This indicated that both clathrin-independent endocytosis and macropinocytosis likely contribute to the uptake of PMG5 by BMDCs.

### PMG5 Thermal Stability

A differential scanning calorimetry (DSC) thermogram of PLGA indicated that the polymer had a glass transition temperature of ˜45° C. (representing the amorphous region of PLGA) (FIG. 22A). Adding the PAMAM G5 to PLGA (i.e., physical mixture) had no effect on the thermal properties of the PLGA. When PLGA was incorporated into NPs (with and without sucrose) using the nanoprecipitation technique it did not exhibit any changes in thermal properties when compared to unprocessed PLGA; except for a slight shift in the glass transition temperature to ˜49° C. The DSC thermogram demonstrated that the PMG5 formulation is thermally stable at the ambient temperature since it did not display any thermal events in the range 0-49° C. It is likely that the highly positive surface charge of PMG5 helped to prevent aggregation in solution at room temperature due to repulsive forces (FIG. 22B). At 37° C., PMG5 dispersed in the water showed no significant change in size, PDI, and surface charge for at least 10 days of stirring at 300 rpm (FIGS. 22B-D); however, after 10 days, the hydrodynamic size (and PDI) did increase most likely due to aggregation.

### Dendritic Cell Activation

Having established that BMDCs can take up PMG5 (FIG. 15D) he impact PMG5 has on the activation/maturation of BMDCs was evaluated. Thus, BMDCs were incubated with PMG5 and cell surface expression of activation markers (CD80, CD40, and MHC class 1) was evaluated. PMG5 did not cause a significant increase in MHC class I or CD80 expression vs untreated BMDCs whether in the presence or absence of Ad5-TRP2 (FIGS. 16A and 16B), however, PMG5 did cause a significant increase in CD40 expression (FIG. 16C). CD40 (on DCs) plays a role in stimulating antigen-specific CD8+ T cell immune responses indirectly upon interaction with CD40L on CD4+ T cells, or directly through interaction with CD40L on CD8+ T cells. In an effort to discern the type of immune response (Th1 versus Th2) that may be promoted upon PMG5 interacting with BMDCs, the levels of secretion of two cytokines (IL-6 and IP-10) by BMDCs cultured with PMG5 were evaluated. While IL-4 is a potent Th2 cytokine promoting naïve T cells towards a Th2 biased phenotype, DCs do not secrete IL-4 thus IL-6 was chosen as reports indicate secretion of IL-6 from DCs cause secretion of IL-4 from T cells, steering them towards a Th2 phenotype. IP-10 (also known as CXCL10) was chosen because it has demonstrated effectiveness as a chemoattractant for Th1 cells and has been shown to be important for Th1 based antitumor activity. It was found that PMG5 did not cause significant increases in secretion of either IP-10 and IL-6, in the presence or absence of Ad5-TRP2 while the TLR-9 agonist, CpG ODN (a positive control), promoted significant increases in secretion of both cytokines (FIGS. 16D and 16E).

Antitumor Efficacy of Ad5-TRP2 Combined with PMG5

Initially, prophylactic studies were performed and demonstrated that vaccinating non-tumor challenged mice with Ad5-TRP2 at 5×10⁷, 1×10⁸, or 5×10⁸ PFU led to significantly enhanced levels of TRP2-specific CD8+T lymphocytes in the peripheral blood compared to unvaccinated mice on day 14 post-vaccination (FIG. 17A). No significant differences were seen between the different doses although a trend toward a dose-dependent response was recognized. However, unpaired t-tests revealed there to be a significant difference between 5×10⁷ and 5×10⁸ PFU vaccinations (p=0.031) but no significance when 1×10⁸ and 5×10⁸ PFU vaccinations were compared (p=0.098). These same mice were also challenged subcutaneously (SC) with live B16.F10 cells (1×10⁵ cells/mouse) on day 14 post-vaccination and all three vaccination doses showed a protective phenotype with all control mice (100%: n=5) succumbing to the tumor by day 20 post-tumor challenge whilst the mice vaccinated with 5×10⁷, 1×10⁸ and 5×10⁸ PFU Ad5-TRP2 demonstrated 60%, 60% and 80% tumor-free survival at the termination of the experiment on day 55 respectively (FIG. 17B).

In a therapeutic setting, there was a marginal but non-significant increase in TRP2-specific CD3+CD8+T lymphocytes when the two doses of Ad5-TRP2 (1×10⁸ PFU versus (5×10⁸ PFU) were compared (FIG. 23) as was also observed in the prophylactic setting (FIG. 17A). Thus, for subsequent therapeutic studies, mice were vaccinated with 1×10⁸ PFUs Ad5-TRP2 unless otherwise stated. To evaluate the effect of PMG5 on Ad5-TRP2 vaccination therapeutically, mice were challenged SC with B16.F10 cells on day 0 and then, on day 1 post tumor challenge (PTC), vaccinated SC with 1×10⁸ PFU Ad5-TRP2 in the contralateral flank, followed by peritumor administration of PMG5 on days 8, 11, 13 PTC. FIG. 18H demonstrates that mice in the Ad5-TRP2/PMG5 treatment group had a significant increase in the percent of TRP2-specific CD3+CD8+T lymphocytes in the peripheral blood versus mice in the Ad5-TRP2 treatment group; (2.08±0.73% vs 1.21 t 0.28% respectively (p=0.0176)). In comparison, mice treated with Ad5-TRP2/CpG ODN had only 0.96±0.31% TRP2-specific CD3+CD8+T lymphocytes in their peripheral blood (p=0.0073: compared to Ad5-TRP2/PMG5 treatment group). Mice treated with Ad5-TRP2/PMG5 also exhibited reduced tumor growth rates compared to naïve mice (FIG. 18A vs FIG. 18E) with 5/9 mice remaining tumor-free 90 days PTC (after a secondary tumor challenge at 60 days PTC). Mice treated with Ad5-TRP2/CpG had only 2/9 mice remaining tumor-free 90 days PTC. When compared to all other treatment groups the Ad5-TRP2/PMG5 group provided the most protection against tumor growth. This ultimately translated into Ad5-TRP2/PMG5 mice being the only group with a significant increase in median survival of mice when compared to the Ad5-TRP2 mice (103 days for Ad5-TRP2/PMG5 treated mice versus 35 days naïve mice (P=0.014)) (FIG. 18G). To evaluate if treatments given to mice generated immunological memory protecting mice from future tumor challenges, surviving mice that were tumor-free (TF) at day 60 were rechallenged with B16.F10 cells (FIG. 18I). Ad5-TRP2/PMG5 TF mice (5) did not develop tumors 30 days after tumor rechallenge. In addition, Ad5-TRP2/CpG TF mice (2) also did not develop tumors within this time frame (FIG. 18I). The treatments demonstrated no signs of toxicity as determined by weight measurements and monitoring for signs of distress (FIG. 18J).

In-Vitro Evaluation of PM Made with Generations 3 and 4 PAMAM

Given the antitumor efficacy observed when mice were treated with Ad5-TRP2/PMG5, PM variants were made using different generations of PAMAM (generation 3 and 4, ethylenediamine core). Given the size-dependent uptake of PMG5 formulations, the particle characteristics of these PM formulations were also evaluated. The hydrodynamic diameter of the PM seems to increase with decreasing generation i.e., PM made using generation 3 PAMAM (PMG3) had a hydrodynamic diameter of 231.68±2.36 nm while PM made using generation 4 PAMAM (PMG4) had a hydrodynamic diameter of 172.36±3.01 nm (FIG. 24). The surface charges of PMG3 and PMG4 were +39.24±0.56 mV and +42.6±0.64 mV, respectively (FIG. 24D). The PMG4 and PMG3 also proved to be stable with small changes in size and surface charge after 30 days in Nanopure water at 37° C. and room temperature (RT) (FIGS. 25A and D). The PDI of both PMG3 and PMG4 appeared to be stable at RT for 40 days. However, for PMG4 incubating beyond 10 days at 37° C., the PDI progressively increased (FIG. 25E). Evaluating the uptake of PMG3, PMG4, PMG5 by BMDCs demonstrates that PMG5 is taken up to a significantly greater degree (as indicated by increased MFI of BMDCs) than PMG3 (8.13±0.63 vs 5.46±0.67, p=0.0002) and marginally more (but not statistically significant) than PMG4 (8.93±0.63 vs 8.13±0.30, p=0.314) (FIG. 33).

Antitumor Efficacy of PMG3. PMG4, and PMG5 with ICM in Therapeutic Ad5-TRP2 Vaccinated Mice

The antitumor efficacy of PMG3 and PMG4 in therapeutically vaccinated mice was evaluated similar to how PMG5 was tested. The antitumor efficacy observed with Ad5-TRP2/PMG5 was greater than Ad5-TRP2/PMG3 and Ad5-TRP2/PMG4 (FIG. 19C, D, E). All mice treated with Ad5-TRP2/PMG3 (10/10 mice) succumbed to tumor burden by day 30 PTC while 8/10 mice treated with Ad5-TRP2/PMG4 succumbed to tumor burden by day 45 PTC. This is in stark contrast to mice treated with Ad5-TRP2/PMG5 treated mice where significantly delayed tumor growth is observed in mice that do develop tumors (4/9) and 5/9 mice did not develop tumors. This translated to mice treated with Ad5-TRP2/PMG3 or Ad5-TRP2/PMG4 having a median survival of 30 days and 33 days PTC respectively, while for Ad5-TRP2/PMG5 treated mice the median survival was greater than 100 days PTC (FIG. 19H). The survival percentages by the end of the study (day 60 PTC) for mice treated with Ad5-TRP2/PMG5, Ad5-TRP2/PMG4, or Ad5-TRP2/PMG3 were 66.6%, 20%, and 0%, respectively (FIG. 19G). The level of TRP2-specific CD3+CD8+T lymphocytes also progressively increased in mice treated with PM formulations made from increasing generations of PAMAM in vaccinated mice: Ad5-TRP2/PMG3 (1.147%±0.28), Ad5-TRP2/PMG4 (1.554%±0.71) and Ad5-TRP2/PMG5 (2.076%±0.73) (FIG. 19F). This indicates that the antitumor effects of PMG5 in therapeutically vaccinated mice are unique to the PMG5 particle formulation and may be due to their size and/or charge which differed significantly when compared to PMG3 and PMG4 (FIG. 24).

Antibodies specific for immune checkpoint proteins, whether they be antagonist antibodies that block immunosuppressive pathways such as anti-PD1 and anti-CTLA4 or agonist antibodies that trigger T cell activation and proliferation such as anti-41BB, have demonstrated significant success in cancer immunotherapy either clinically or in preclinical settings, respectively. Given their dependence on tumor-specific T cells for efficacy, and the demonstrated synergy between inhibiting T cell exhaustion by administering anti-PD1 and promoting T cell survival and proliferation with anti-41BB, the effects of this combination were evaluated when combined with the Ad5-TRP2 cancer vaccine and PMG3, PMG4, or PMG5. Before launching into these studies it was established that the combination of anti-PD1 and anti-4-1BB was more efficient at promoting TRP2-specific immune responses and enhancing survival of B16.F10-challenged mice when combined with Ad5-TRP2 compared to either ICM antibody alone (FIG. 27). Mice vaccinated with Ad5-TRP2 and subsequently treated with anti-PD1/anti-4-1BB demonstrated significantly higher levels of TRP2-specific CD3+CD8+T lymphocytes in the peripheral blood (7.94±3.41%) compared to Ad5-TRP-2 alone; however, the addition of any of the PM formulations to the combination of Ad5-TRP2 and ICM did not result in any significant changes in levels of TRP2-specific CD3+CD8+T lymphocytes when compared to Ad-TRP2+anti-PD1/anti-41BB (FIG. 20F).

Interestingly, when compared to the survival of mice treated with Ad5-TRP2 plus either PMG3 or PMG5 (FIG. 19) there was a substantial improvement in survival when ICM was included (FIG. 20) such that the survival rates were similar to that obtained for mice treated with Ad5-TRP2/PMG5 alone (FIG. 19). The addition of ICM to the Ad5-TRP2/PMG5 group did not further enhance survival (FIG. 20). To elaborate, at 100 days PTC, mice treated with Ad5-TRP2/PMG3 ICM demonstrated 70% survival vs 0% survival for mice treated with Ad5-TRP2/PMG3; mice treated with Ad5-TRP2/PMG4 ICM demonstrated 60% survival vs 20% survival for mice treated with Ad5-TRP2/PMG4, and finally, mice treated with Ad5-TRP2/PMG5 ICM had 50% survival vs 55% survival for mice treated with Ad5-TRP2/PMG5. These findings did not reveal statistically significant differences between the survival curves of Ad5-TRP2/PMG3 ICM vs Ad5-TRP2/PMG4 ICM vs Ad5-TRP2/PMG5 ICM. The increase in anti-tumor efficacy (for Ad5-TRP2/PMG3 ICM and Ad5-TRP2/PMG4 ICM) vs Ad5-TRP2 ICM alone (as indicated by increased survival) occurred without a concomitant increase in levels of TRP2-specific CD3+CD8+T lymphocytes in the blood: Ad5-TRP2/PMG3 ICM (6.543%±4.87%), Ad5-TRP2/PMG4 ICM (6.651%±4.32%) vs Ad5-TRP2 ICM (7.9%±3.25%). This indicates the effects of this increased antitumor efficacy may be due to the effects of the particles on the local tumor milieu that make the tumor cells more vulnerable to tumor cell killing by T cells but not through promotion of T cell proliferation. Combining PM formulations and ICM in Ad5-TRP2 vaccinated mice did not result in any observable signs of toxicity to the mice as there was no obvious decrease in mice weights (FIG. 29).

### Evaluating the Effects of Immune Cell Populations on the Ad5-TRP2/PMG5 ICM Treatment Group

As thus far observed, PMG5 significantly increases the effects of the Ad5-TRP2 vaccine in a therapeutic setting, while other PM formulations either have no effect (PMG3) or a modest increase in survival (PMG4). However, when ICM is included, Ad5-TRP2/PMG5 ICM does not seem to provide an increase in efficacy vs Ad5-TRP2/PMG5 while ICM added to Ad5-TRP2/PMG3 or Ad5-TRP2/PMG4 increased survival of each entity at least additively. In an effort to elucidate which cells are responsible for the efficacy of the Ad5-TRP2/PMG5 treatment group, these mice were depleted of select lymphocyte populations (NK cells, CD4+ T cells, CD8+ T cells, or CD4+/CD8+ T cells). As a comparison, mice treated with Ad5-TRP2/PMG5 ICM were also were depleted of the same select lymphocyte populations. FIG. 21A demonstrates that for mice treated with Ad5-TRP2/PMG5 both adaptive immune cell populations (CD4+ T cells, CD8+ T cells) tested influenced the observed anti-tumor efficacy as indicated by decreased survival (although only CD8 T cell depletion resulted in statistically significant differences). Depleting NK cells showed a trend towards reducing the antitumor efficacy, but this decrease was not statistically significant from the Ad5-TRP2/PMG5 survival curve. At 80 days PTC mice treated with Ad5-TRP2/PMG5 displayed 80% survival (4/5 mice); depleting NK cells resulted in 40% survival (2/5 mice); and depleting CD4+ T cells resulted in 20% survival (1/5 mice). Depleting CD8+ T cells completely abrogated the antitumor efficacy of Ad5-TRP2/PMG5 treatment with all mice succumbing to tumors by day 40 PTC. In contrast. Ad5-TRP2/PMG5 ICM-treated mice appeared to have relied exclusively on CD8+ T cells in mediating anti-tumor efficacy. On day 80 PTC, mice treated with Ad5-TRP2/PMG5 ICM demonstrated 60% survival (3/5 mice) while depleting CD4+ T cells or NK cells resulted in 80% survival (4/5 mice). However, depleting CD8+ T cells resulted in 5/5 mice succumbing to tumors on day 30 and depleting both CD8 and CD4 resulting in 5/5 mice succumbing to tumors by day 26 PTC. Taking these results into account it points to Ad5-TRP2/PMG5 mice possibly relying on both the adaptive and innate immune system to elicit its antitumor efficacy and supplementing Ad5-TRP2/PMG5 treatment with ICM shifts the response to being solely reliant on CD8+ T cells at mediating an effector response.

### Discussion

Cancer vaccines hold enormous potential for the treatment of intractable tumors such as late-stage melanoma. However, despite promising results in preclinical settings for cancer vaccines per se, their effectiveness in the clinic has not been demonstrated especially when compared to other cancer immunotherapeutic modalities such as immune checkpoint therapy, and adoptive T cell therapy. This is likely due in part to the immunosuppressive nature of the TME and thus tackling such immunosuppressive obstacles on potentially more than one front in combination with cancer vaccine administration may provide synergistic therapeutic benefit. Several lines of evidence support the feasibility of this type of approach. For instance, it has been previously demonstrated that local delivery of adjuvants such as CpG (type B) initiate in-situ immunization and combining anti-OX40 immune checkpoint modulation with intratumoral CpG (type B) administration abrogates tumor growth and protects mice from tumor rechallenge. Previous studies have demonstrated that intratumoral administration of CpG (type B) in combination with therapeutic vaccination with an adenovirus cancer vaccine encoding a model tumor antigen increases its antitumor efficacy resulting in increased survival. The administration of CpG was shown to increase the levels of tumor-specific T cells and reduce the levels of Tregs within the tumor. Recently, it has been shown that intratumoral administration of CMP-001, a virus-like particle encapsulating CpG (type A), in combination with systemic administration of immune checkpoint blockade therapy provided increased protection in mice vs mice treated with anti-PD1 alone. These studies indicate that: 1) delivery of adjuvants to the site of the tumor can at least partially abrogate the immunosuppressive phenotype of the tumor thus enabling function to tumor-specific CTLs, including those generated by a cancer vaccine; 2) combining this approach with immune checkpoint modulation therapy (i.e. agonist antibodies and/or antagonist antibodies) and therefore dampening the immunosuppressive potency within the TME has the potential to synergistically increase the antitumor efficacy of therapeutic cancer vaccines. Thus, these approaches were brought together in a multipronged strategy to generate optimal tumor-specific immune responses in a therapeutically relevant tumor model using a murine melanoma cancer vaccine. Late-stage melanoma is a major health concern with a 9-19% 5-year survival rate and has shown to be responsive to immunotherapies such as immune checkpoint modulation in clinical settings. Here an attenuated serotype 5 adenovirus was employed that encoded for a TAA, TRP2, as the cancer vaccine in a poorly immunogenic B16.F10 murine melanoma model. TSAs admittedly may be better at generating an immune response; however, TSAs lack the possibility of being prepared on a large standardized scale as they are often patient- or even tumor-specific; and there is still the issue of the immunosuppressive TME. Also, given the lack of immunogenicity associated with TAAs, a cancer vaccine strategy that generates a substantial immune response against TAAs more than likely generates the same if not greater response should TSAs be implemented instead. This approach using a poorly immunogenic TAA, to treat a murine melanoma model which has been noted to be poorly responsive to immunotherapy will serve as a rigorous testing platform that mimics the clinical situation with respect to a lack of significant antitumor efficacy being demonstrated when the cancer vaccine is administered alone.

The prophylactic studies presented demonstrated the capability of the Ad5-TRP2 vaccine to trigger a detectable TRP2-specific CD8+T lymphocyte response and that the resulting immune response possessed antitumor activity as it led to significant protection of mice challenged with B16.F10 cells (FIG. 17). Based on previous studies this protection is likely to have been mediated by CD4+T lymphocytes and CD8+T lymphocytes contributing to the priming and effector stages, respectively. B16.F10 cells as evidenced by the tumor volume graphs) grow very aggressively and so an early time point (day 1 PTC) was chosen to commence the Ad5-TRP2 vaccination treatment in the therapeutic studies. Despite being able to protect mice from B16F10 tumor challenge in a prophylactic setting, Ad5-TRP2 was unable to significantly improve mouse survival in a therapeutic setting (FIG. 18I). To improve the antitumor efficacy of Ad5-TRP2 local administrations of a nano-formulated adjuvant, PMG5, were introduced. PMG5 is a cationic nanoscale adjuvant that serves as a platform to increase the efficacy of immunotherapeutic modalities. Characterization of PMG5 indicates it is thermally stable below 50° C. and stable in an aqueous solution for at least 10 days without significant change in size and surface charge (FIG. 22). The size of PMG5 can also be optimized to efficiently enter BMDCs (55). The combination of PMG5 of Ad5-TRP2 as a therapeutically treatment in B16.F10-challenged mice elicited higher levels of TRP2-specific CD3+CD8+T lymphocytes in the PBL than mice treated with Ad5-TRP2 alone (FIG. 18). These mice also demonstrated significantly higher survival and reduced tumor burden and protection from future tumor challenges, implying immunological memory was generated. These effects were not noted in mice that were unvaccinated and received PMG5 alone. An increase in efficacy of the Ad5-TRP2 vaccine was not noted when soluble CpG (type B) was used as a treatment instead of PMG5; as evidenced by lower levels of TRP2-specific CD3+CD8+T lymphocytes (FIG. 18H) and lower median survival (FIG. 5G). The antitumor efficacy of PMG5 differed from particles of a different polymer composition (PMG3 and PMG4) which were not as efficient in eliciting a similar antitumor efficacy when administered in combination with Ad5-TRP2 (FIG. 19). Taken together this demonstrates that, PMG5 can serve as a potent adjuvant in vivo since it can increase the efficacy of a cancer vaccine. It has previously been demonstrated that TRP2₁₈₀₋₁₈₈-specific CD3+CD8+T lymphocytes activated by adenoviral-based vaccines (encoding TRP2) are responsible for melanocyte-specific killing and therefore very likely to have played a role in the killing of the B16.F10 cells in the therapeutic model used here.

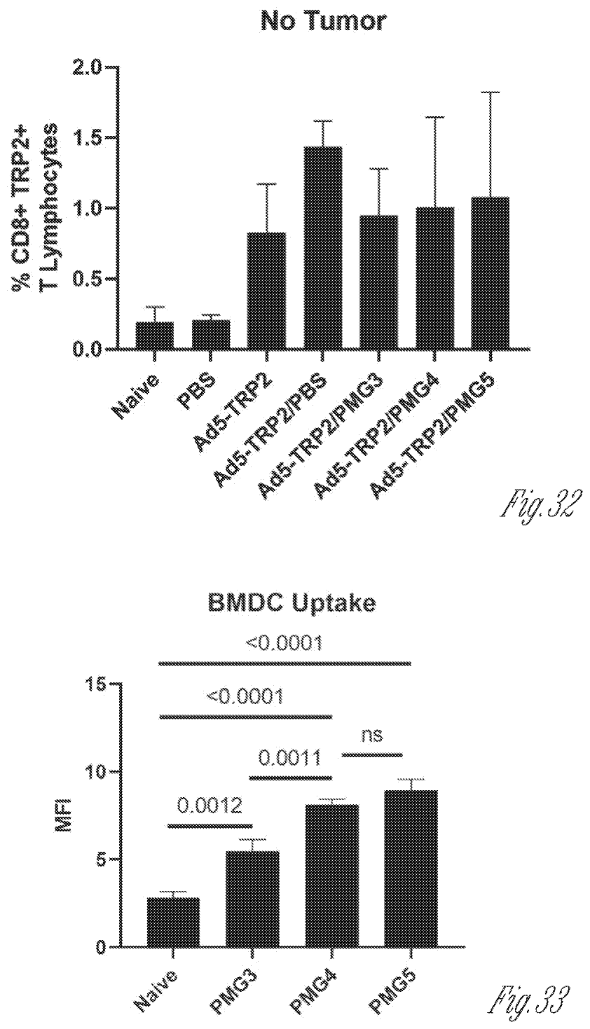

Thus, the increase in efficacy when PMG5 was administered was likely due to the effect PMG5 had on the TME allowing for an increase in cytotoxicity and/or proliferation of TRP2₁₈₀₋₁₈₈-specific CD3+CD8+T lymphocytes. In the absence of a tumor, there was no significant change in the levels of TRP2-specific CD3+CD8+T lymphocytes in the PBL suggesting that the PMG5 had an influence on the TME rather than directly affecting the efficacy of the cancer vaccine (FIG. 32). One possibility is that the administration of PMG5 increases the homing of activated T lymphocytes to the tumor due to inflammatory chemokine release (57). Another possibility is that the differences in efficacy between PMG5 vs PMG4 and PMG3 maybe due to the difference in the size of these particle formulations (FIG. 24) and their resultant differences in the ability to traffic to the lymph nodes and interact with immune cell populations there.

Combining Ad5-TRP2/PMG3 or Ad5-TRP2/PMG4 with ICM leads to an increase, albeit not statistically significant, in the median survival of mice vs Ad5-TRP2/ICM alone (FIG. 20G). The trending increase in the overall survival of mice treated with this multipronged therapy occurred without a concomitant increase in the levels of TRP2-specific CD3+CD8+T lymphocytes in the peripheral blood indicating the efficacy was likely due to the effects PM formulations had on the TME and/or its surroundings (FIG. 20F). Combining Ad5-TRP2/PMG5 with ICM also did not result in an increase in TRP2-specific CD3+CD8+T lymphocytes in the peripheral blood vs Ad5-TRP2/ICM (FIG. 20F), however, unlike the findings for Ad5-TRP2/PMG3 and Ad5-TRP2/PMG4, there was no improvement in the overall survival of mice treated with Ad5-TRP2/PMG5 ICM vs Ad5-TRP2/PMG5 (FIGS. 19G and 19H vs FIGS. 20G and 20H). The reasons for the discrepancy in findings when comparing PMG5 to either PMG3 or PMG4 are not readily apparent; however, the differences in size and surface charge observed cannot be ruled out as a contributing factor. It is possible that PMG5 affected the TME in such a way that addition of ICM (which can also affect the TME) is redundant. Further studies will be required to determine if this, or some other explanation, is valid.

The lack of antitumor efficacy of PMG5 in unvaccinated mice indicates the efficacy of the Ad5-TRP2/PMG5 treatment was not due to tumor cell death directly induced by PMG5 but possibly an immunological effect relating to the innate arm of the immune system that creates a favorable environment for CTL activity. Depletion studies tentatively, though not conclusively, indicate this where Ad5-TRP2/PMG5 treated mice appeared to possess a broader reliance on a diverse array of immune cells tested (NK cells, CD4+ and CD8+ T cells), while Ad5-TRP2/PMG5 ICM treated mice relied predominantly on CD8+ T cells to mediate an antitumor effector response. It is possible that because of this reliance on multiple cell types (in Ad5-TRP2/PMG5 treated mice), individually depleting one of these cell types (e.g. NK cells), while having an apparent impact on survival, did not attain statistical significance because of the contribution of other cell types (e.g. CD4+ and CD8+ T cells). Upon addition of ICM, we propose that the tumor-specific CD8+ T cells no longer required assistance from CD4+ T cells and NK cells to mediate the antitumor effect; and that ICM was providing (or blocking) the signals to CD8+ T cells required to promote an optimal effector response. PMG5 also caused an increase in the expression of CD40 by BMDCs in vitro (FIG. 16C). The CD40-CD40L signaling pathway exists between DCs, T cells, macrophages, and NK cells. CD40 signaling to DCs can result in upregulation of MHC molecules, CD86, and proinflammatory cytokine production. PMG5 also demonstrated increased uptake in BMDCs vs PMG4 and PMG3 (FIG. 33) indicating the ability of PMG5 to be taken up by immune cell populations such as DCs and elicit downstream signals which are possibly similar to, or overlap with, the signaling prompted by ICM thus potentially rendering the treatments redundant. Whilst PMG3 and PMG4 potentially lack the capacity to provide these downstream signals that affect immune efficacy given their decreased uptake by BMDCs.

It is important to put the results obtained into the broader context of cancer immunotherapy. Combining a single administration of an adenovirus-based cancer vaccine with 3 administrations of PMG5 provides durable antitumor immune responses and protects mice from tumor rechallenge. Previous preclinical vaccination studies in mice have demonstrated the ability of Ad5-TRP2 and Ad5-mTRP2 to protect against B16.F10 metastases, however, their ability to effectively combat solid B16.F10 tumors in a therapeutic setting has rarely been investigated possibly because B16.F10 solid tumors are notoriously difficult to eradicate with cancer vaccines in general. Successful attempts have been recorded using multiple doses and high titers of Ad2-mTRP2 or using a multipronged non-viral based vaccine regimen. Additional studies have demonstrated the efficacy of combining immune checkpoint agonists and antagonists. This may be the first demonstration of combining a local administration of a cationic nanoparticulate adjuvant formulation and a cancer vaccine with positive results.

In summary, a cationic NP formulation was prepared using PLGA and PAMAM (PM). Combining a single administration of Ad5-TRP2 with subsequent peritumoral administrations with PMG5 significantly increased the efficacy of the Ad5-TRP2 cancer vaccine in treating the murine melanoma model. This treatment regimen not only stops tumor growth but protects mice from subsequent tumor challenges. This is evident from the elevated TRP2-specific CD8+ T cell response, the significant increase in median survival, and prolonged survival of mice receiving this treatment regimen. Combining underperforming PM formulations with ICM results in a significant increase in the efficacy of the treatments. This demonstrates that this formulation can be combined with other immunotherapy modalities to yield additional antitumor efficacies.

### REFERENCES

Anderson et al., Gene Ther., 7:1034 (2000).
Araújo et al., Molecules, 23:2849 (2018).
Aznar et al., J. ImmunoTherapy Cancer, 7:116 (2019).
Banchereau & Steinman, Nature, 92:245 (1998).
Biswas et al., Biomaterials, 34:1289 (2013).
Blank et al., American Journal of Respiratory Cell and Molecular Biology, 49:67 (2013).
Bono et al., ACS Omega, 4:6796 (2019).
Buonaguro et al., Clin. Vaccine Immunol., 18:23 (2011).
Caminati et al., J. Am. Chem. Soc. 112:8515 (1990).
Carreau & Pavlick, Surgical Oncology, _:_(2019).
Carver & Schnitzer, Nature Reviews Cancer, 3:571 (2003).
Chauhan & Kaul, J. Nanopart. Res., 22:226 (2018).
Chen et al., Cancer Immunol. Res., 3:149 (2015).
Cheng et al., PLOS Pathogens, 3:e25 (2007).
Cook et al., Proc. Natl. Acad. Sci. U.S.A., 109:9977 (2012).
Dai et al., Clin. Cancer Res., 21:1127 (2015).
Decker et al., Frontiers Immunol., 8:_(2017).
Dobrzanski, Frontiers Oncol., 3:_(2013).
Dufès et al., Adv. Drug Del. Rev., 57:2177 (2005).
Dufour et al., J. Immunol., 168:3195 (2002).
Foged et al., Int. J. Pharmac., 298:315 (2005).
Fougeroux & Hoist, Int. J. Mol. Sci., 18:686 (2017).
Geary et al., Cancer Immunol. Immunoth., 60:1309 (2011).
Gershenwald et al., J. Clin. Oncol., 26:9035 (2008).
Hollingsworth & Jansen, Vaccines, 4:7 (2019).
Hollingsworth & Jansen, Vaccines, 605:4(1) (2019).
Intra & Salem, J. Pharma. Sci., 99:368 (2010).
Kantoff et al., N. Eng. J. Med. 383:411 (2010).
Karan, Vaccine, 35:5794 (2017).
Kim & Kim, Korean J. Intern. Med., 33:483 (2018).
Konopka et al, Polymers (Basel), 10:540 (2018).
Krishnamoorthy et al., J. Immun., 178:S181 (2007).
Labieniec-Watala & Watala, J. Pharma. Sci., 104:2 (2015).
Ledgerwood et al., Vaccine, 22:304 (2010).
Lemke-Miltner et al., J. Immunol., 204:1386 (2020).
Lou et al., J. Immunother., 34:279 (2011).
Ma & Clark, Semin. Immunol., 21:265 (2009).
Miller et al., Cancer Res., 62:5260 (2002).
Mohsen et al., WIREs Nanom. Nanob., 12:e1579 (2020).
Mougel et al., Frontiers in Immunology, 10:467 (2019).
Moynihan et al., Nat. Med., 22:1402 (2016).
National Cancer Institute: Surveillance, Epidemiology, and End Results Program (SEER) Cancer 679 Stat Facts: Melanoma of the Skin. 2019 [Available from: 680 https://seer.cancer.gov/statfacts/html/melan.html.
Pedersen et al., J. Immunol., 191:3955 (2013).
Perricone et al., Mol. Ther., 1:275 (2000).
Polak et al., Human Immunol., 70:331 (2009).
Rajagopalan et al., Am. J. Cardio., 90:512 (2002).
Rakhmilevich et al., Int. Rev. Immunol., 31:267 (2012).
Sadekar & Ghandehari, Adv. Drug Del. Rev., 64:571 (2012).
Sagiv-Barfi et al., Sci. Transl. Med., 10:eaan4488 (2018).
Santos et al., Mol. Pharma., 7:763 (2010).
Seydoux et al., Int. J. Nanomedicine, 9: 3885 (2014).
Siemens et al., J. Immunol., 166:731 (2001).
Smith et al., Adv. Mater. Tech., 4:1800349 (2019).
Steitz et al., Cancer Gene Ther., 13:318 (2006).
Steitz et al., Gene Ther., 8:1255 (2001).
Steitz et al., Int. J. Cancer, 88:89 (2000).
Steitz et al., J. Invest. Dermatol., 124:144 (2005).
Svenson & Tomalia, Adv. Drug Del. Rev., 7:2106 (2005).
Swain, Current Biol., 5:849 (1995).
Taub et al., J. Exo. Med. 177:1809 (1993).
Thiagarajan et al., Eur. J. Pharma. Biopharm., 84:330 (2013).
Thomas et al., Biomaterials, 35:814 (2014).
Tolcher et al., Clin. Cancer Res., 23:5349 (2017).
Tsai et al., Biomacromolecules, 12:4283 (2011).
Veglia & Gabrilovich, Curr. Opin. Immun., 45:43 (2017).
Wade et al., Fresenius' J. Anal. Chem., 369:378 (2001).
Wafa et al., Acta Biomater., 50:417 (2017).
Wafa et al., Biomacromolecules, _:_(2019c).
Wafa et al., J. Pharmacol. Exp. Ther. 370:855 (2019a).
Wafa et al., Nanomedicine, 21:102055 (2019b).
Wamock et al., “Introduction to Viral Vectors” in Viral Vectors for Gene Therapy: Methods and Protocols, O.-W. Merten, M. Al-Rubeai, Eds. (Humana Press, Totowa, NJ), pp. 1-25 (2011).
Wamock et al., In: Merten O-W, Al-Rubeai M, 612 editors. Viral Vectors for Gene Therapy: Methods and Protocols. Totowa, NJ: Humana Press; 2011. p. 1-613 25.
Wei et al., Oncoimmunology, 3:e28248 (2014).
Wróbel et al., J. Clin. Med., 8:368 (2019).

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A cationic nanoparticle formed of a biodegradable or biocompatible synthetic polymer and a dendrimer and having a diameter of about 125 nm to 1000 nm.

2. The nanoparticle of claim 1 wherein the synthetic polymer comprises a polyolefin, silicone, polyacrylate, polyester, polyether, polyamide, polyurethane, polyvinyl alcohol, polyethyleneimine, polyglycolic acid, polyhydroxyalkanoate, polylactic acid, or co-polymers of polyglycolic acid and poly-lactic.

3. (canceled)

4. The nanoparticle of claim 1 wherein the dendrimer comprises a polyamidoamine, polyethyleneimine, polypropyleneimine, poly-L-lysine, diethylaminoethyl dextran, polyvinyl alcohol, polyethylene glycol or carbosilane.

5. (canceled)

6. The nanoparticle of claim 1 which does not include isolated nucleic acid.

7. The nanoparticle of claim 1 wherein the diameter of the nanoparticle is about 150 nm to about 600 nm, about 150 nm to about 250 nm or about 250 nm to about 500 nm.

8-9. (canceled)

10. (canceled)

11. The nanoparticle of claim 1 wherein the dendrimer is PAMAM.

12. The nanoparticle of claim 11 wherein the PAMAM is G2, G3, G4, G5, G6 or G7.

13. A lyophilized product comprising a plurality of the nanoparticles of claim 1 optionally comprising a cryoprotectant or carrier.

14. (canceled)

15. A pharmaceutical composition comprising an amount of the nanoparticle of claim 1 effective as an adjuvant which optionally comprises a cryoprotectant.

16. A method to enhance an immune response in a mammal, comprising administering to the mammal an amount of an immunogen or a vector encoding the immunogen and an amount of the nanoparticle of claim 1.

17. The method of claim 16 wherein the mammal is a human.

18. The method of claim 16 wherein the vector is a viral vector.

19. The method of claim 18 wherein the viral vector is an adenovirus, lentivirus, retrovirus, herpesvirus or adeno-associated viral vector.

20. The method of claim 16 wherein the mammal has cancer.

21. The method of claim 20 wherein the cancer is melanoma or a solid tumor.

22. (canceled)

23. The method of claim 16 wherein the immunogen is a tumor-specific antigen, a tumor-associated antigen or microbial antigen.

24-26. (canceled)

27. The method of claim 16 wherein the immunogen or vector is administered at a site that is different than the administration site for the nanoparticles.

28-29. (canceled)

30. The method of claim 16 further comprising administering one or more chemotherapeutic agents or one or more checkpoint inhibitors.

31. (canceled)
